(19) United States
(12) Patent Application Publication
(10) Pub. No.: US 2024/0327428 A1
(43) Pub. Date: Oct. 3, 2024

(54) Inhibitors of human immunodeficiency virus replication, method of making and method of using thereof

(71) Applicant: ASCLETIS BIOSCIENCE CO., LTD., Hangzhou (CN)

(72) Inventors: Jinzi Jason WU, Hangzhou (CN); Bin LIANG, Hangzhou (CN); Bailing YANG, Hangzhou (CN)

(21) Appl. No.: 18/585,050

(22) Filed: Feb. 22, 2024

Related U.S. Application Data
(60) Provisional application No. 63/489,668, filed on Mar. 10, 2023.

(30) Foreign Application Priority Data
Feb. 23, 2023 (CN) 202310197203.3

(51) Int. Cl. C07D 513/04; A61K 31/4355; A61K 31/4365; A61K 31/4375; A61K 31/4439; A61K 31/519; A61K 31/53; A61K 31/5377; A61K 45/06; A61P 31/18; C07D 401/14; C07D 471/04; C07D 487/04; C07D 491/048; C07D 495/04; C07D 498/04; C07D 519/00

(52) U.S. Cl. CPC C07D 513/04; A61K 31/4355; A61K 31/4365; A61K 31/4375; A61K 31/4439; A61K 31/519; A61K 31/53; A61K 31/5377; A61K 45/06; A61P 31/18; C07D 401/14; C07D 471/04; C07D 487/04; C07D 491/048; C07D 495/04; C07D 498/04; C07D 519/00

(57) ABSTRACT

The present application describes compounds of capsid inhibitors that are useful for treating HIV infection in a human. One aspect of the present application relates to a compound of Formula I, stereoisomers thereof, pharmaceutically acceptable salts thereof, or deuterium substitutes thereof as described in the present disclosure.

### RELATED APPLICATIONS

This application claims priority to U.S. Provisional Application No. 63/489,668, filed Mar. 10, 2023 and to Chinese Application No. CN202310197203.3, filed Feb. 23, 2023, which are incorporated herein by reference in their entirety.

### FIELD

The invention relates to compounds, compositions, and methods for the treatment of human immunodeficiency virus (HIV) infection. More particularly, the invention provides novel inhibitors of HIV, pharmaceutical compositions containing such compounds, and methods for using these compounds in the treatment of HIV infection. The invention also relates to methods for making the compounds hereinafter described.

### BACKGROUND

Acquired immunodeficiency syndrome (AIDS) is the result of infection by HIV. Current therapy for HIV-infected individuals consists of a combination of approved anti-retroviral agents. Close to four dozen drugs are currently approved for HIV infection, either as single agents, fixed dose combinations or single tablet regimens; the latter two containing 2-4 approved agents. These agents belong to a number of different classes, targeting either a viral enzyme or the function of a viral protein during the virus replication cycle. Thus, agents are classified as either nucleotide reverse transcriptase inhibitors (NRTIs), non-nucleotide reverse transcriptase inhibitors (NNRTIs), protease inhibitors (Pis), integrase strand transfer inhibitors (INSTIs), or entry inhibitors (one, maraviroc, targets the host CCR5 protein, while the other, enfuvirtide, is a peptide that targets the gp41 region of the viral gp160 protein). In addition, a pharmacokinetic enhancer (cobicistat or ritonavir) can be used in combinations with antiretroviral agents (ARVs) that require boosting.

Despite the armamentarium of agents and drug combinations, there remains a medical need for new anti-retroviral agents. High viral heterogeneity, drug-associated toxicity, tolerability problems, and poor adherence can all lead to treatment failure and may result in the selection of viruses with mutations that confer resistance to one or more antiretroviral agents or even multiple drugs from an entire class.

As a result, new drugs are needed that are easier to take, have high genetic barriers to the development of resistance, and have improved safety over current agents. Also needed are new formulations and methods of treatment which utilize these compounds.

### SUMMARY

One aspect of the present application relates to a compound of Formula I:

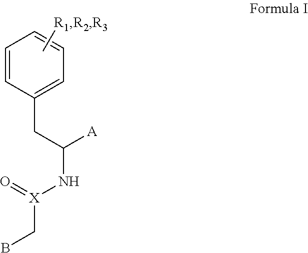

or stereoisomers thereof, or pharmaceutically acceptable salts thereof, or deuterium substitutes thereof, wherein
wherein each of R₁, R₂, and R₃ is independently selected from the group consisting of H, Cl, F, -OMe, —CN, C₁-C₃ alkyl, and C₃-C₅ cycloalkyl, wherein the C₁-C₃ alkyl is optionally substituted with 1-3 halo;
wherein X is C, S, S═O, or P—R₁₀;
wherein R₁₀ is selected from the group consisting of H, OH, C₁-C₃ alkyl, C₃-C₅ cycloalkyl, —O—C₁-C₃ alkyl, —O—C₃-C₅ cycloalkyl, —NH—C₁-C₃ alkyl, and —NH—C₃-C₅ cycloalkyl;
wherein A is

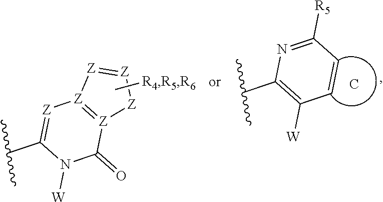

wherein each Z is independently selected from the group consisting of C, CH, N, NH, O, and S;
wherein each of R₄, R₅ and R₆ is independently selected from the group consisting of H, halogen, C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, C₃₋₁₅ cycloalkyl, heterocyclyl, C₆₋₁₀ aryl, 5-10 membered heteroaryl, oxo, —NO₂, —N₃, —CN, —NH(C₁₋₉ alkyl), —NH(C₁₋₈ haloalkyl), —NH(C₂₋₆ alkenyl), —NH(C₂₋₆ alkynyl), —NH(C₃₋₁₅ cycloalkyl), —NH(heterocyclyl), —NH(C₆₋₁₀ aryl), —NH(5-10 membered heteroaryl), —N(cycloalkyl)₂, —N(C₁₋₉ alkyl)₂, —N(C₁₋₈ haloalkyl)₂, —N(C₂₋₆ alkenyl)₂, —N(C₂₋₆ alkynyl)₂, —N(C₃₋₁₅ cycloalkyl)₂, —N(heterocyclyl)₂, —N(C₆₋₁₀ aryl)₂, —N(5-10 membered heteroaryl)₂, —N(C₁₋₉ alkyl)(C₁₋₈ haloalkyl), —N(C₁₋₉ alkyl)(C₂₋₆ alkenyl), —N(C₁₋₉ alkyl)(C₂₋₆ alkynyl), —N(C₁₋₉ alkyl)(C₃₋₁₅ cycloalkyl), —N(C₁₋₉ alkyl)(heterocyclyl), —N(C₁₋₉ alkyl)(C₆₋₁₀ aryl), —N(C₁₋₉ alkyl)(5-10 membered heteroaryl), —SH, —O(C₁₋₉ alkyl), —O(C₁₋₈ haloalkyl), —O(C₂₋₆ alkenyl), —O(C₂₋₆ alkynyl), —O(C₃₋₁₅ cycloalkyl), —O(heterocyclyl), —O(C₆₋₁₀ aryl), and —O(5-10 membered heteroaryl);
wherein each of R₄, R₅ and R₆ is optionally substituted with one or more substituents each independently selected from the group consisting of halo, C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, C₃₋₁₅ cycloalkyl, heterocyclyl, C₆₋₁₀ aryl, 5-10 membered heteroaryl, oxo, —NO₂, —N₃, —CN, —S(O)— C₁₋₉ alkyl, —S(O)₂— C₁₋₉ alkyl, —S(O)—C₃₋₁₅ cycloalkyl, and —S(O)₂— C₃₋₁₅ cycloalkyl, wherein the C₁₋₉ alkyl and C₃₋₁₅ cycloalkyl are optionally substituted with one or more halo;
wherein ring C is 5-10 membered heteroaryl; wherein the 5-10 membered heteroaryl is optionally substituted with one or more substituents each independently selected from the group consisting of halo, C₁₋₉ alkyl, C₁₋₈ haloalkyl, C₁₋₆ alkoxy, and C₁₋₆ haloalkoxy;
wherein W is

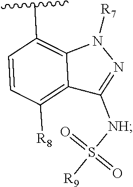

wherein R₇ is C₁-C₃ alkyl, or C₃₋₅ cycloalkyl; wherein R₇ is optionally substituted with one to nine halo;
wherein R₈ is selected from the group consisting of H, Cl, F, -OMe, —CN, —C₁-C₃ alkyl, and —C₃-C₅ cycloalkyl, wherein the C₁-C₃ alkyl is optionally substituted with 1-3 fluorines;
wherein R₉ is C₁-C₃ alkyl, or C₃-C₅ cycloalkyl; wherein R₉ is optionally substituted with 1-3 fluorines; and
wherein B is

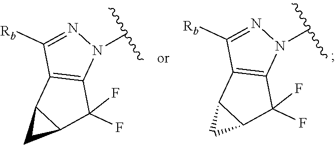

wherein R_{b} is C₁-C₃ alkyl, or C₃₋₅ cycloalkyl; wherein R_{b} is optionally substituted with one to nine halo.

In another aspect, A is selected from the group consisting of

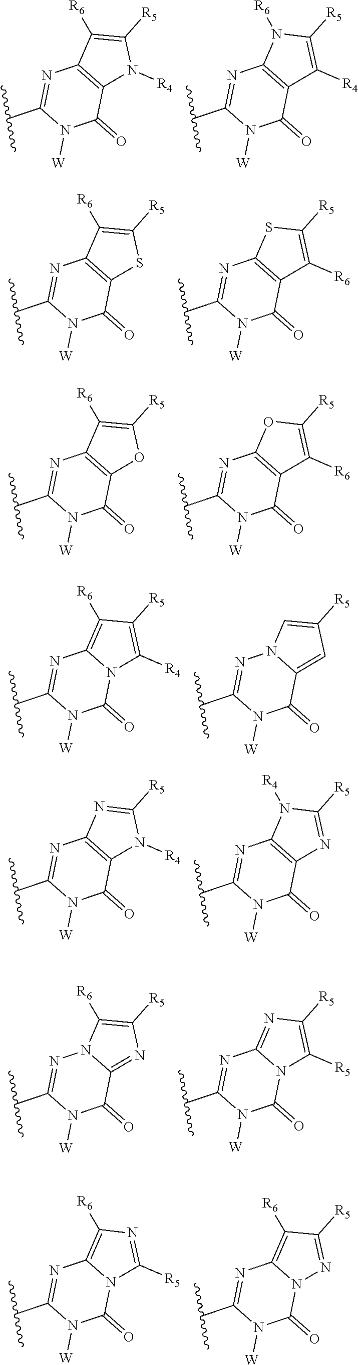

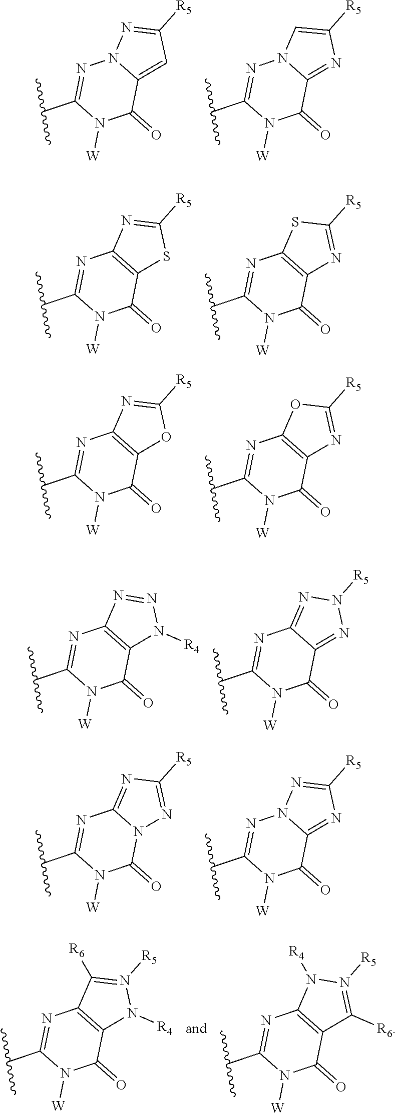

In another aspect, the present application discloses a composition comprising a compound of Formula I, or stereoisomers thereof, or pharmaceutically acceptable salt thereof, or deuterated compounds thereof.

In another aspect, the present application discloses a method of treating HIV infection comprising administering a composition comprising a compound of Formula I, or stereoisomers thereof, or pharmaceutically acceptable salt thereof, or deuterated compounds thereof to a patient.

In another aspect, the present application discloses a compound of Formula (I) or stereoisomers thereof, or pharmaceutically acceptable salt thereof, or deuterated compounds thereof for use in therapy.

In another aspect, the present application discloses a compound of Formula (I) or stereoisomers thereof, or pharmaceutically acceptable salt thereof, or deuterated compounds thereof for use in treating HIV infection.

In another aspect, the present application discloses the use of a compound of Formula (I), or stereoisomers thereof, or pharmaceutically acceptable salt thereof, or deuterated compounds thereof in the manufacture of a medicament for the treatment of HIV infection.

### DETAILED DESCRIPTION

### I. Definitions

Unless defined otherwise, all technical and scientific terms used herein have the same meaning as commonly understood by one of ordinary skill in the art. A dash at the front or end of a chemical group is a matter of convenience to indicate the point of attachment to a parent moiety; chemical groups may be depicted with or without one or more dashes without losing their ordinary meaning. A prefix such as “Cᵤ₋ᵥ” or “Cᵤ-Cᵥ” indicates that the following group has from u to v carbon atoms, where u and v are integers. For example, “C₁₋₆ alkyl” or “C₁-C₆ alkyl” indicates that the alkyl group has from 1 to 6 carbon atoms.

The term “alkyl” is a monovalent or divalent linear or branched saturated hydrocarbon radical. For example, an alkyl group can have 1 to 10 carbon atoms (i.e., C₁₋₁₀ alkyl) or 1 to 8 carbon atoms (i.e., C₁₋₈ alkyl) or 1 to 6 carbon atoms (i.e., C₁₋₆ alkyl) or 1 to 4 carbon atoms (i.e., C₁₋₄ alkyl). Examples of alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-Propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, and octyl (—(CH₂)₇CH₃). Alkyl groups can be unsubstituted or substituted.

The term “alkenyl” is a monovalent or divalent linear or branched hydrocarbon radical with at least one carbon-carbon double bond. For example, an alkenyl group can have 2 to 8 carbon atoms (i.e., C₂₋₈ alkenyl) or 2 to 6 carbon atoms (i.e., C₂₋₆ alkenyl) or 2 to 4 carbon atoms (i.e., C₂₋₄ alkenyl). Examples of alkenyl groups include, but are not limited to, ethenyl (—CH═CH₂), allyl (—CH₂CH═CH₂), and —CH₂—CH═CH—CH₃. Alkenyl groups can be unsubstituted or substituted.

The term “alkynyl” is a monovalent or divalent linear or branched hydrocarbon radical with at least one carbon-carbon triple bond. For example, an alkynyl group can have 2 to 8 carbon atoms (i.e., C₂₋₈ alkynyl) or 2 to 6 carbon atoms (i.e., C₂₋₆ alkynyl) or 2 to 4 carbon atoms (i.e., C₂₋₄ alkynyl). Examples of alkynyl groups include, but are not limited to, acetylenyl (—C≡CH), propargyl (—CH₂C≡CH), and —CH₂-C≡C—CH₃. Alkynyl groups can be unsubstituted or substituted.

The terms “halogen” or “halo” refers to fluoro (—F), chloro (—Cl), bromo (—Br) and iodo (—I).

The term “haloalkyl” is an alkyl as defined herein, wherein one or more hydrogen atoms of the alkyl are independently replaced by a halogen, which may be the same or different, such that the alkyl is divalent. The alkyl group and the halogen can be any of those described above. In some embodiments, the haloalkyl defines the number of carbon atoms in the alkyl portion, e.g., C₁₋₄ haloalkyl includes CF₃, CH₂F, CHF₂, CH₂CF₃, CH₂CH₂CF₃, CCl₂CH₂CH₂CH₃, and C(CH₃)₂(CF₂H). Haloalkyl groups can be unsubstituted or substituted.

The term “alkoxy” refers to the group —O-alkyl, where alkyl is as defined above. For example, C₁₋₄ alkoxy refers to an —O-alkyl group having 1 to 4 carbons. Alkoxy groups can be unsubstituted or substituted.

The term “haloalkoxy” is an alkoxy as defined herein, wherein one or more hydrogen atoms of the alkyl in the alkyoxy are independently replaced by a halogen, which may be the same or different, such that the alkyl is divalent. The alkoxy group and the halogen can be any of those described above. In some embodiments, the haloalkoxy defines the number of carbon atoms in the alkyl portion, e.g., C₁₋₄ haloalkoxy includes OCF₃, OCH₂F, OCH₂CF₃, OCH₂CH₂CF₃, OCCl₂CH₂CH₂CH₃, and OC(CH₃)₂(CF₂H). Haloalkoxy groups can be unsubstituted or substituted.

The term “cycloalkyl” is a monovalent or divalent single all carbon ring or a multiple condensed all carbon ring system wherein the ring in each instance is a non-aromatic saturated or unsaturated ring. For example, in some embodiments, a cycloalkyl group has 3 to 12 carbon atoms, 3 to 10 carbon atoms, 3 to 8 carbon atoms, 3 to 6 carbon atoms, 3 to 5 carbon atoms, or 3 to 4 carbon atoms. Exemplary single ring cycloalkyl groups include cyclopropyl, cyclobutyl, cyclopentyl, cyclohexyl, cyclohexenyl, cycloheptyl, cycloheptenyl, and cyclooctyl. Cycloalkyl also includes multiple condensed ring systems (e.g., ring systems comprising 2 rings) having about 7 to 12 carbon atoms. The rings of the multiple condensed ring system can be connected to each other via fused, spiro, or bridged bonds when allowed by valency requirements. Exemplary multiple ring cycloalkyl groups include octahydropentalene, bicyclo[2. 2. 1]heptane, bicyclo[2. 2. 2]octane, bicyclo[2. 2. 2]oct-2-ene, and spiro[2. 5]octane. Cycloalkyl groups can be unsubstituted or substituted.

The term “aryl” as used herein refers to a monovalent or divalent single all carbon aromatic ring or a multiple condensed all carbon ring system wherein the ring is aromatic. For example, in some embodiments, an aryl group has 6 to 20 carbon atoms, 6 to 14 carbon atoms, 6 to 12 carbon atoms, or 6 to 10 carbon atoms. Aryl includes a phenyl radical. Aryl also includes multiple condensed ring systems (e.g., ring systems comprising 2, 3 or 4 rings) having about 9 to 20 carbon atoms in which multiple rings are aromatic. The rings of the multiple condensed ring system can be connected to each other via fused bonds when allowed by valency requirements. It is also understood that when reference is made to a certain atom-range membered aryl (e.g., 6-10 membered aryl), the atom range is for the total ring atoms of the aryl. For example, a 6-membered aryl would include phenyl and a 10-membered aryl would include naphthyl. Non-limiting examples of aryl groups include, but are not limited to, phenyl, naphthyl, anthracenyl, and the like. Aryl groups can be unsubstituted or substituted.

The term “alkylaryl” refers to an alkyl as defined herein, wherein one or more hydrogen atoms of the alkyl are independently replaced by an aryl group, which may be the same or different. The alkyl group and the aryl group can be any of those described above, such that the alkyl is divalent. In some embodiments, an alkylaryl group has 7 to 24 carbon atoms, 7 to 16 carbon atoms, 7 to 13 carbon atoms, or 7 to 11 carbon atoms. An alkylaryl group defined by the number of carbon atoms refers to the total number of carbon atoms present in the constitutive alkyl and aryl groups combined. For example, C₇ alkylaryl refers to benzyl, while C₁₁ alkylaryl includes 1-methylnaphthyl and n-pentylphenyl. In some embodiments the number of carbon atoms in the alkyl and aryl portion can be designated separately, e.g., C₁₋₆ alkyl-C₆₋₁₀ aryl. Non-limiting examples of alkylaryl groups include, but are not limited to, benzyl, 2,2-dimethylphenyl, n-pentylphenyl, 1-methylnaphthyl, 2-ethylnaphthyl, and the like. Alkylaryl groups can be unsubstituted or substituted.

The terms “heterocyclyl” or “heterocycle” or “heterocycloalkyl” as used herein refers to a single saturated or partially unsaturated non-aromatic ring or a non-aromatic multiple ring system that has at least one heteroatom in the ring (i.e., at least one annular (i.e., ring-shaped) heteroatom selected from oxygen, nitrogen, and sulfur). Unless otherwise specified, a heterocyclyl group has from 3 to about 20 annular atoms, for example from 3 to 12 annular atoms, for example from 4 to 12 annular atoms, 4 to 10 annular atoms, or 3 to 8 annular atoms, or 3 to 6 annular atoms, or 3 to 5 annular atoms, or 4 to 6 annular atoms, or 4 to 5 annular atoms. Thus, the term includes single saturated or partially unsaturated rings (e.g., 3, 4, 5, 6 or 7-membered rings) having from about 1 to 6 annular carbon atoms and from about 1 to 3 annular heteroatoms selected from the group consisting of oxygen, nitrogen and sulfur in the ring. The rings of the multiple condensed ring (e.g. bicyclic heterocyclyl) system can be connected to each other via fused, spiro and bridged bonds when allowed by valency requirements. Heterocycles include, but are not limited to, azetidine, aziridine, imidazolidine, morpholine, oxirane (epoxide), oxetane, thietane, piperazine, piperidine, pyrazolidine, piperidine, pyrrolidine, pyrrolidinone, tetrahydrofuran, tetrahydrothiophene, dihydropyridine, tetrahydropyridine, quinuclidine, 2-oxa-6-azaspiro[3. 3]heptan-6-yl, 6-oxa-1-azaspiro[3. 3]heptan-1-yl, 2-thia-6-azaspiro[3. 3]heptan-6-yl, 2,6-diazaspiro[3. 3]heptan-2-yl, 2-azabicyclo[3. 1. 0]hexan-2-yl, 3-azabicyclo[3. 1. 0]hexanyl, 2-azabicyclo[2. 1. 1]hexanyl, 2-azabicyclo[2. 2. 1]heptan-2-yl, 4-azaspiro[2. 4]heptanyl, 5-azaspiro[2. 4]heptanyl, and the like. Heterocyclyl groups can be unsubstituted or substituted.

The terms “5-10 membered heteroaryl” or “heteroaryl” refers to a single aromatic ring that has at least one atom other than carbon in the ring, wherein the atom is selected from the group consisting of oxygen, nitrogen and sulfur; “5-10 membered heteroaryl” also includes multiple condensed ring systems that have at least one such aromatic ring, which multiple condensed ring systems are further described below. Thus, “5-10 membered heteroaryl” includes single aromatic rings of from about 1 to 6 carbon atoms and about 1-4 heteroatoms selected from the group consisting of oxygen, nitrogen and sulfur. The sulfur and nitrogen atoms may also be present in an oxidized form provided the ring is aromatic. Exemplary 5-10 membered heteroaryl ring systems include but are not limited to pyridyl, pyrimidinyl, oxazolyl or furyl. “5-10 membered heteroaryl” also includes multiple condensed ring systems (e.g., ring systems comprising 2, 3 or 4 rings) wherein a 5-10 membered heteroaryl group, as defined above, is condensed with one or more rings selected from 5-10 membered heteroaryls (to form for example 1,8-naphthyridinyl) and aryls (to form, for example, benzimidazolyl or indazolyl) to form the multiple condensed ring system. Thus, a 5-10 membered heteroaryl (a single aromatic ring or multiple condensed ring system) can have about 1-20 carbon atoms and about 1-6 heteroatoms within the 5-10 membered heteroaryl ring. For example, tetrazolyl has 1 carbon atom and 4 nitrogen heteroatoms within the ring. The rings of the multiple condensed ring system can be connected to each other via fused bonds when allowed by valency requirements. It is to be understood that the individual rings of the multiple condensed ring system may be connected in any order relative to one another. It is to be understood that the point of attachment for a 5-10 membered heteroaryl or 5-10 membered heteroaryl multiple condensed ring system can be at any suitable atom of the 5-10 membered heteroaryl or 5-10 membered heteroaryl multiple condensed ring system including a carbon atom and a heteroatom (e.g., a nitrogen). It also to be understood that when a reference is made to a certain atom-range membered (e.g., a 5-10 membered heteroaryl), the atom range is for the total ring atoms of the 5-10 membered heteroaryl and includes carbon atoms and heteroatoms. It is also to be understood that the rings of the multiple condensed ring system may include an aryl ring fused to a heterocyclic ring with saturated or partially unsaturated bonds (e.g., 3, 4, 5, 6 or 7-membered rings) having from about 1 to 6 annular carbon atoms and from about 1 to 3 annular heteroatoms selected from the group consisting of oxygen, nitrogen and sulfur in the ring. For example, a 5-10 membered heteroaryl includes thiazolyl and a 5-10 membered heteroaryl includes quinolinyl. Exemplary 5-10 membered heteroaryls include but are not limited to pyridyl, pyrrolyl, pyrazinyl, pyrimidinyl, pyridazinyl, pyrazolyl, thienyl, indolyl, imidazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, quinazolyl, benzofuranyl, benzimidazolyl, thianaphthenyl, pyrrolo[2,3-b]pyridinyl, quinazolinyl-4(3H)-one, triazolyl, and tetrazolyl. 5-10 membered heteroaryl groups can be unsubstituted or substituted.

The term “alkylheteroaryl” refers to an alkyl as defined herein, wherein one or more hydrogen atoms of the alkyl are independently replaced by a heteroaryl group, which may be the same or different, such that the alkyl is divalent. The alkyl group and the heteroaryl group can be any of those described above. In some embodiments, the number of atoms in the alkyl and heteroaryl portion are designated separately, e.g., C₁₋₆ alkyl-5 to 10 membered heteroaryl having one to four heteroatoms each independently N, O, or S. Alkylheteroaryl groups can be unsubstituted or substituted.

The term “oxo” as used herein refers to ═O.

The term “substituted” as used herein refers to wherein one or more hydrogen atoms of the group are independently replaced by one or more substituents (e.g., 1, 2, 3, or 4 or more) as indicated.

The term “optionally” means that the subsequently described event or condition may or may not occur, and the specification includes situations in which the event or condition occurs and situations in which it does not occur. For example, the term “optionally substituted alkyl” includes both unsubstituted alkyl and substituted alkyl.

The term “compound of the present application” includes compounds disclosed herein, for example a compound of the present application includes compounds of Formula I, including the compounds of the Examples. In some embodiments, a “compound of the present application” includes compounds of Formula I.

The term “pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

The terms “therapeutically effective amount” or “effective amount” as used herein refers to an amount that is effective to elicit the desired biological or medical response, including the amount of a compound that, when administered to a subject for treating a disease, is sufficient to affect such treatment for the disease. The effective amount will vary depending on the compound, the disease, and its severity and the age, weight, etc., of the subject to be treated. The effective amount can include a range of amounts. As is understood in the art, an effective amount may be in one or more doses, i.e., a single dose or multiple doses may be required to achieve the desired treatment endpoint. An effective amount may be considered in the context of administering one or more therapeutic agents, and a single agent may be considered to be given in an effective amount if, in conjunction with one or more other agents, a desirable or beneficial result may be or is achieved. Suitable doses of any co-administered compounds may optionally be lowered due to the combined action (e.g., additive or synergistic effects) of the compounds.

The term “co-administration” as used herein refers to administration of unit dosages of the compounds disclosed herein before or after administration of unit dosages of one or more additional therapeutic agents, for example, administration of the compound disclosed herein within seconds, minutes, or hours of the administration of one or more additional therapeutic agents. For example, in some embodiments, a unit dose of a compound of the present application is administered first, followed within seconds or minutes by administration of a unit dose of one or more additional therapeutic agents. Alternatively, in other embodiments, a unit dose of one or more additional therapeutic agents is administered first, followed by administration of a unit dose of a compound of the present application within seconds or minutes. In some embodiments, a unit dose of a compound of the present application is administered first, followed, after a period of hours (e.g., 1-12 hours), by administration of a unit dose of one or more additional therapeutic agents. In other embodiments, a unit dose of one or more additional therapeutic agents is administered first, followed, after a period of hours (e.g., 1-12 hours), by administration of a unit dose of a compound of the present application. Co-administration of a compound disclosed herein with one or more additional therapeutic agents generally refers to simultaneous or sequential administration of a compound disclosed herein and one or more additional therapeutic agents, such that therapeutically effective amounts of each agent are present in the body of the subject.

Provided are also pharmaceutically acceptable salts, hydrates, solvates, tautomeric forms, polymorphs, and prodrugs of the compounds described herein.

The terms “pharmaceutically acceptable” or “physiologically acceptable” refer to compounds, salts, compositions, dosage forms and other materials which are useful in preparing a pharmaceutical composition that is suitable for veterinary or human pharmaceutical use.

The compounds described herein may be prepared and/or formulated as pharmaceutically acceptable salts or when appropriate as a free base. Pharmaceutically acceptable salts are non-toxic salts of a free base form of a compound that possesses the desired pharmacological activity of the free base. These salts may be derived from inorganic or organic acids or bases. For example, a compound that contains a basic nitrogen may be prepared as a pharmaceutically acceptable salt by contacting the compound with an inorganic or organic acid. Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in Remington: The Science and Practice of Pharmacy, 21st Edition, Lippincott Wiliams and Wilkins, Philadelphia, Pa., 2006.

Examples of “pharmaceutically acceptable salts” of the compounds disclosed herein also include salts derived from an appropriate base, such as an alkali metal (for example, sodium, potassium), an alkaline earth metal (for example, magnesium), ammonium and N(C₁-C₄ alkyl)₄_{⁺}. Also included are base addition salts, such as sodium or potassium salts.

Provided are also compounds described herein or pharmaceutically acceptable salts, isomers, or a mixture thereof, in which from 1 to n hydrogen atoms attached to a carbon atom may be replaced by a deuterium atom or D, in which n is the number of hydrogen atoms in the molecule. As known in the art, the deuterium atom is a non-radioactive isotope of the hydrogen atom. Such compounds may increase resistance to metabolism, and thus may be useful for increasing the half-life of the compounds described herein or pharmaceutically acceptable salts, isomer, or a mixture thereof when administered to a mammal. See, e.g., Foster, “Deuterium Isotope Effects in Studies of Drug Metabolism”, Trends Pharmacol. Sci., 5(12):524-527 (1984).

Such compounds are synthesized by means well known in the art, for example by employing starting materials in which one or more hydrogen atoms have been replaced by deuterium.

Examples of isotopes that can be incorporated into the disclosed compounds also include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, 150, 170, ¹⁸O, ³¹P, ³²P, ³⁵S, ¹⁸F, ³⁶Cl, ¹²³I, and ¹²⁵I, respectively. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of Formula I-A-1 can generally be prepared by conventional techniques known to those skilled in the art or by processes analogous to those described in the Examples as set out below using an appropriate isotopically-labeled reagent in place of the non-labeled reagent previously employed.

The compounds of the embodiments disclosed herein, or their pharmaceutically acceptable salts may contain one or more asymmetric centers and may thus give rise to enantiomers, tautomer, diastereomers, and other stereoisomeric forms that may be defined, in terms of absolute stereochemistry, as (R)- or (S)- or, as (D)- or (L)- for amino acids, as well as deuterated analogs thereof. The chemical formula shown in the present application is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included. Where compounds are represented in their chiral form, it is understood that the embodiment encompasses, but is not limited to, the specific diastereomerically or enantiomerically enriched form. Where chirality is not specified but is present, it is understood that the embodiment is directed to either the specific diastereomerically or enantiomerically enriched form; or a racemic or scalemic mixture of such compound(s). As used herein, “scalemic mixture” is a mixture of stereoisomers at a ratio other than 1:1.

The term “stereoisomer” as used herein refers to a compound made up of the same atoms bonded by the same bonds but having different three-dimensional structures, which are not interchangeable. The present application contemplates various stereoisomers and mixtures thereof and includes “enantiomers”, which refers to two stereoisomers whose molecules are non-superimposable mirror images of one another.

The term “tautomer” as used herein refers to a proton shift from one atom of a molecule to another atom of the same molecule. In some embodiments, the present application includes tautomers of said compounds.

The term “solvate” as used herein refers to the result of the interaction of a solvent and a compound. Solvates of salts of the compounds described herein are also provided. Hydrates of the compounds described herein are also provided.

The term “hydrate” as used herein refers to a compound of the disclosure that is chemically associated with one or more molecules of water.

The terms “prevention” or “preventing” means any treatment of a disease or condition that causes the clinical symptoms of the disease or condition not to develop. Compounds may, in some embodiments, be administered to a subject (including a human) who is at risk or has a family history of the disease or condition.

The term “prodrug” as used herein refers to a derivative of a drug that upon administration to the human body is converted to the parent drug according to some chemical or enzymatic pathway. In some embodiments, a prodrug is a biologically inactive derivative of a drug that upon administration to the human body is converted to the biologically active parent drug according to some chemical or enzymatic pathway.

The terms “treatment” or “treat” or “treating” as used herein refers to an approach for obtaining beneficial or desired results. For purposes of the present application, beneficial or desired results include, but are not limited to, alleviation of a symptom and/or diminishment of the extent of a symptom and/or preventing a worsening of a symptom associated with a disease or condition. In one embodiment, “treatment” or “treating” includes one or more of the following: a) inhibiting the disease or condition (e.g., decreasing one or more symptoms resulting from the disease or condition, and/or diminishing the extent of the disease or condition); b) slowing or arresting the development of one or more symptoms associated with the disease or condition (e.g., stabilizing the disease or condition, delaying the worsening or progression of the disease or condition); and c) relieving the disease or condition, e.g., causing the regression of clinical symptoms, ameliorating the disease state, delaying the progression of the disease, increasing the quality of life, and/or prolonging survival. “At risk individual” as used herein refers to an individual who is at risk of developing a condition to be treated. An individual “at risk” may or may not have detectable disease or condition, and may or may not have displayed detectable disease prior to the treatment of methods described herein. “At risk” denotes that an individual has one or more so-called risk factors, which are measurable parameters that correlate with development of a disease or condition and are known in the art. An individual having one or more of these risk factors has a higher probability of developing the disease or condition than an individual without these risk factor(s).

### II. Compounds

One aspect of the present application relates to a compound of Formula I:

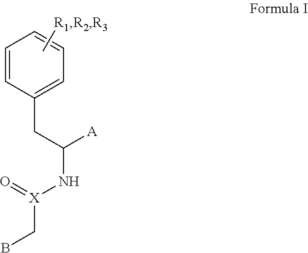

or stereoisomers thereof, or pharmaceutically acceptable salts thereof, or deuterium substitutes thereof,
wherein each of R₁, R₂, and R₃ is independently selected from the group consisting of H, Cl, F, -OMe, —CN, C₁-C₃ alkyl, and C₃-C₅ cycloalkyl, wherein the C₁-C₃ alkyl is optionally substituted with 1-3 halo;
wherein X is C, S, S═O, or P—R₁₀;
wherein R₁₀ is selected from the group consisting of H, OH, C₁-C₃ alkyl, C₃-C₅ cycloalkyl, —O—C₁-C₃ alkyl, —O—C₃-C₅ cycloalkyl, —NH—C₁-C₃ alkyl, and —NH—C₃-C₅ cycloalkyl;
wherein A is

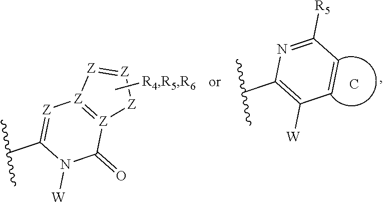

wherein each Z is independently selected from the group consisting of C, CH, N, NH, O, and S;
wherein each of R₄, R₅ and R₆ is independently selected from the group consisting of H, halogen, C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, C₃₋₁₅ cycloalkyl, heterocyclyl, C₆₋₁₀ aryl, 5-10 membered heteroaryl, oxo, —NO₂, —N₃, —CN, —NH(C₁₋₉ alkyl), —NH(C₁₋₈ haloalkyl), —NH(C₂₋₆ alkenyl), —NH(C₂₋₆ alkynyl), —NH(C₃₋₁₅ cycloalkyl), —NH(heterocyclyl), —NH(C₆₋₁₀ aryl), —NH(5-10 membered heteroaryl), —N(cycloalkyl)₂, —N(C₁₋₉ alkyl)₂, —N(C₁₋₈ haloalkyl)₂, —N(C₂₋₆ alkenyl)₂, —N(C₂₋₆ alkynyl)₂, —N(C₃₋₁₅ cycloalkyl)₂, —N(heterocyclyl)₂, —N(C₆₋₁₀ aryl)₂, —N(5-10 membered heteroaryl)₂, —N(C₁₋₉ alkyl)(C₁₋₈ haloalkyl), —N(C₁₋₉ alkyl)(C₂₋₆ alkenyl), —N(C₁₋₉ alkyl)(C₂₋₆ alkynyl), —N(C₁₋₉ alkyl)(C₃₋₁₅ cycloalkyl), —N(C₁₋₉ alkyl)(heterocyclyl), —N(C₁₋₉ alkyl)(C₆₋₁₀ aryl), —N(C₁₋₉ alkyl)(5-10 membered heteroaryl), —SH, —O(C₁₋₉ alkyl), —O(C₁₋₈ haloalkyl), —O(C₂₋₆ alkenyl), —O(C₂₋₆ alkynyl), —O(C₃₋₁₅ cycloalkyl), —O(heterocyclyl), —O(C₆₋₁₀ aryl), and —O(5-10 membered heteroaryl);
wherein each of R₄, R₅ and R₆ is optionally substituted with one or more substituents each independently selected from the group consisting of halo, C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, C₃₋₁₅ cycloalkyl, heterocyclyl, C₆₋₁₀ aryl, 5-10 membered heteroaryl, oxo, —NO₂, —N₃, —CN, —S(O)— C₁₋₉ alkyl, —S(O)₂— C₁₋₉ alkyl, —S(O)—C₃₋₁₅ cycloalkyl, and —S(O)₂— C₃₋₁₅ cycloalkyl, wherein the C₁₋₉ alkyl and C₃₋₁₅ cycloalkyl are optionally substituted with one or more halo;
wherein ring C is 5-10 membered heteroaryl; wherein the 5-10 membered heteroaryl is optionally substituted with one or more substituents each independently selected from the group consisting of halo, C₁₋₉ alkyl, C₁₋₈ haloalkyl, C₁₋₆ alkoxy, and C₁₋₆ haloalkoxy;
wherein W is

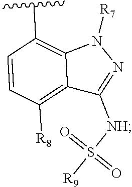

wherein R₇ is C₁-C₃ alkyl, or C₃₋₅ cycloalkyl; wherein R₇ is optionally substituted with one to nine halo;
wherein R₈ is selected from the group consisting of H, Cl, F, -OMe, —CN, —C₁-C₃ alkyl, and —C₃-C₅ cycloalkyl, wherein the C₁-C₃ alkyl is optionally substituted with 1-3 fluorines;
wherein R₉ is C₁-C₃ alkyl, or C₃-C₅ cycloalkyl; wherein R₉ is optionally substituted with 1-3 fluorines; and
wherein B is

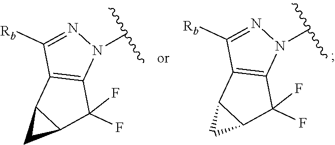

wherein R_{b} is C₁-C₃ alkyl, or C₃₋₅ cycloalkyl; wherein R_{b} is optionally substituted with one to nine halo.

In some embodiments, A is selected from the group consisting of

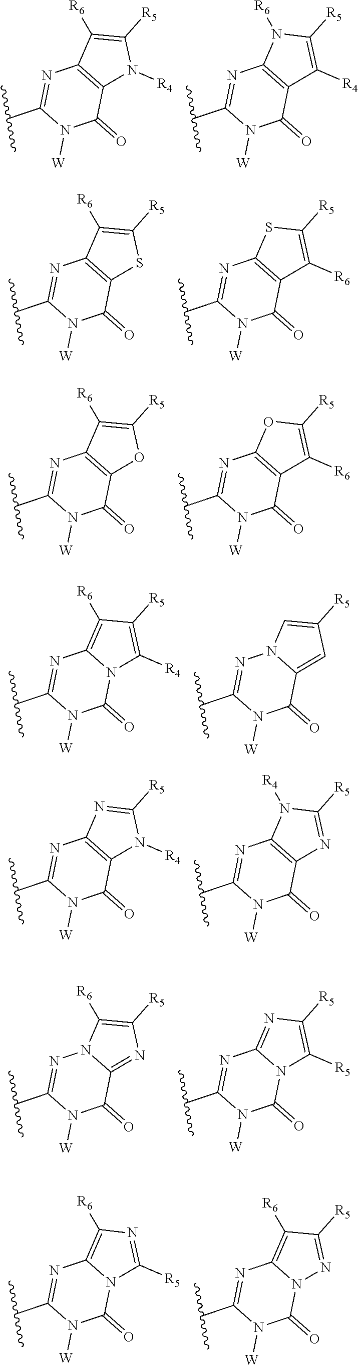

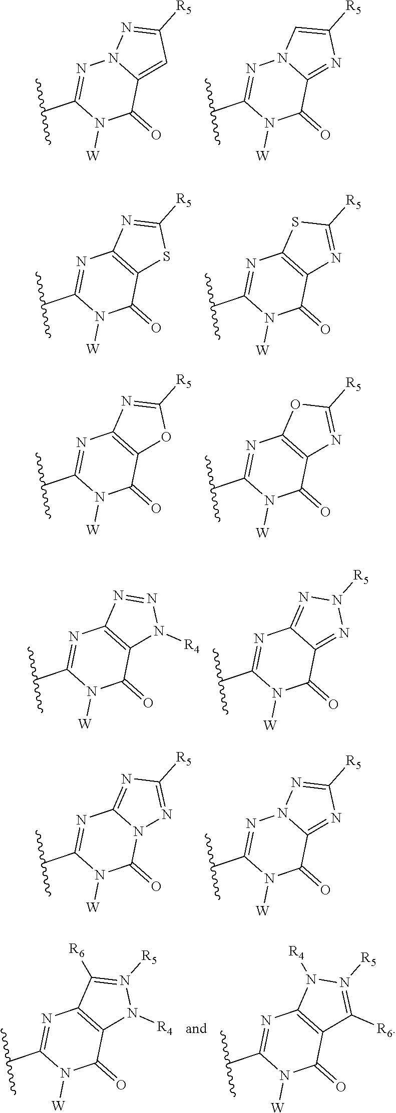

In some embodiments, wherein A is

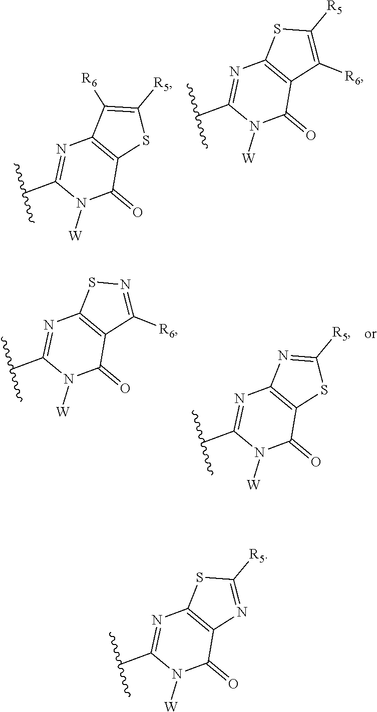

In some embodiments, wherein A is

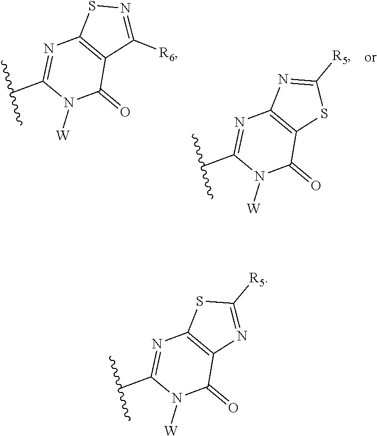

In some embodiments, wherein each of R₁, R₂, and R₃ is independently selected from the group consisting of H, Cl, F.

In some embodiments, wherein X is C.

In some embodiments, wherein B is

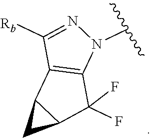

In some embodiments, wherein B is

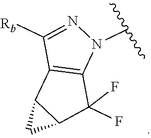

In some embodiments, wherein W is

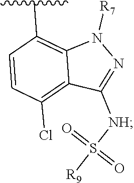

wherein R₇ is C₁-C₃ alkyl, or C₃₋₅ cycloalkyl; wherein R₇ is optionally substituted with one to nine halo;
wherein R₉ is C₁-C₃ alkyl, or C₃-C₅ cycloalkyl; wherein R₉ is optionally substituted with 1-3 fluorines.

In some embodiments, the compound has the structure of Formula II,

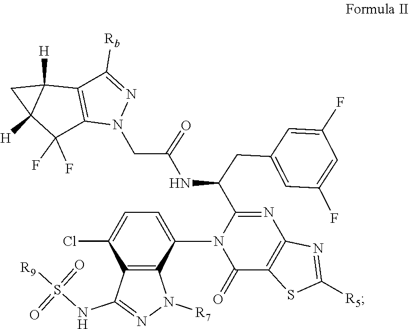

wherein R₇ is C₁-C₃ alkyl, or C₃₋₅ cycloalkyl; wherein R₇ is optionally substituted with one to nine halo;
wherein R₉ is C₁-C₃ alkyl, or C₃-C₅ cycloalkyl; wherein R₉ is optionally substituted with 1-3 fluorines;
wherein R_{b} is C₁-C₃ alkyl, or C₃₋₅ cycloalkyl; wherein R_{b} is optionally substituted with one to nine halo;
wherein R₅ is independently selected from the group consisting of H, halogen, C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, C₃₋₁₅ cycloalkyl, heterocyclyl, C₆₋₁₀ aryl, 5-10 membered heteroaryl, oxo, —NO₂, —N₃, —CN, —NH(C₁₋₉ alkyl), —NH(C₁₋₈ haloalkyl), —NH(C₂₋₆ alkenyl), —NH(C₂₋₆ alkynyl), —NH(C₃₋₁₅ cycloalkyl), —NH(heterocyclyl), —NH(C₆₋₁₀ aryl), —NH(5-10 membered heteroaryl), —N(cycloalkyl)₂, —N(C₁₋₉ alkyl)₂, —N(C₁.s haloalkyl)₂, —N(C₂₋₆ alkenyl)₂, —N(C₂₋₆ alkynyl)₂, —N(C₃₋₁₅ cycloalkyl)₂, —N(heterocyclyl)₂, —N(C₆₋₁₀ aryl)₂, —N(5-10 membered heteroaryl)₂, —N(C₁₋₉ alkyl)(C₁₋₈ haloalkyl), —N(C₁₋₉ alkyl)(C₂₋₆ alkenyl), —N(C₁₋₉ alkyl)(C₂₋₆ alkynyl), —N(C₁₋₉ alkyl)(C₃₋₁₅ cycloalkyl), —N(C₁₋₉ alkyl)(heterocyclyl), —N(C₁₋₉ alkyl)(C₆₋₁₀ aryl), —N(C₁₋₉ alkyl)(5-10 membered heteroaryl), —SH, —O(C₁₋₉ alkyl), —O(C₁₋₈ haloalkyl), —O(C₂₋₆ alkenyl), —O(C₂₋₆ alkynyl), —O(C₃₋₁₅ cycloalkyl), —O(heterocyclyl), —O(C₆₋₁₀ aryl), and —O(5-10 membered heteroaryl);
wherein R₅ is optionally substituted with one or more substituents each independently selected from the group consisting of halo, C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, C₃₋₁₅ cycloalkyl, heterocyclyl, C₆₋₁₀ aryl, 5-10 membered heteroaryl, oxo, —NO₂, —N₃, —CN, —S(O)— C₁₋₉ alkyl, —S(O)₂— C₁₋₉ alkyl, —S(O)— C₃₋₁₅ cycloalkyl, and —S(O)₂— C₃₋₁₅ cycloalkyl, wherein the C₁₋₉ alkyl and C₃₋₁₅ cycloalkyl are optionally substituted with one or more halo.

In some embodiments, wherein R₅ is independently selected from the group consisting of H, halogen, C₁₋₉ alkyl, C₁₋₈ haloalkyl, C₁₋₆ alkoxy, C₁₋₆ haloalkoxy, C₂₋₆ alkoxyalkyl, C₃₋₁₅ cycloalkyl, heterocyclyl, C₆₋₁₀ aryl, 5-10 membered heteroaryl, oxo, —NO₂, —N₃, —CN, —NH(C₁₋₉ alkyl), —NH(C₁₋₈ haloalkyl), —NH(C₂₋₆ alkenyl), —NH(C₂₋₆ alkynyl), —NH(C₃₋₁₅ cycloalkyl), —NH(heterocyclyl), —NH(C₆₋₁₀ aryl), —NH(5-10 membered heteroaryl), —N(cycloalkyl)₂, —N(C₁₋₉ alkyl)₂, —N(C₁₋₈ haloalkyl)₂, —N(C₂₋₆ alkenyl)₂, —N(C₂₋₆ alkynyl)₂, —N(C₃₋₁₅ cycloalkyl)₂, —N(heterocyclyl)₂, —N(C₆₋₁₀ aryl)₂, —N(5-10 membered heteroaryl)₂, —N(C₁₋₉ alkyl)(C₁.s haloalkyl), —N(C₁₋₉ alkyl)(C₂₋₆ alkenyl), —N(C₁₋₉ alkyl)(C₂₋₆ alkynyl), —N(C₁₋₉ alkyl)(C₃₋₁₅ cycloalkyl), —N(C₁₋₉ alkyl)(heterocyclyl), —N(C₁₋₉ alkyl)(C₆₋₁₀ aryl), —N(C₁₋₉ alkyl)(5-10 membered heteroaryl), —O(C₁₋₉ alkyl), —O(C₁₋₈ haloalkyl), —O(C₂₋₆ alkenyl), —O(C₂₋₆ alkynyl), —O(C₃₋₁₅ cycloalkyl), —O(heterocyclyl);
wherein R₅ is optionally substituted with one or more substituents each independently selected from the group consisting of halo, C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, C₃₋₁₅ cycloalkyl, heterocyclyl, C₆₋₁₀ aryl, 5-10 membered heteroaryl, oxo, —NO₂, —N₃, —CN, —S(O)— C₁₋₉ alkyl, —S(O)₂— C₁₋₉ alkyl, —S(O)— C₃₋₁₅ cycloalkyl, and —S(O)₂— C₃₋₁₅ cycloalkyl, wherein the C₁₋₉ alkyl and C₃₋₁₅ cycloalkyl are optionally substituted with one or more halo.

In some embodiments, wherein R₅ is independently selected from the group consisting of H, halogen, C₁₋₉ alkyl, C₁₋₈ haloalkyl, C₁₋₆ alkoxy, C₁₋₆ haloalkoxy, C₂₋₆ alkoxyalkyl, C₃₋₁₅ cycloalkyl, heterocyclyl, C₆₋₁₀ aryl, 5-10 membered heteroaryl, oxo, —NO₂, —N₃, —CN, —NH(C₁₋₉ alkyl), —NH(C₁₋₈ haloalkyl), —NH(C₂₋₆ alkenyl), —NH(C₂₋₆ alkynyl), —NH(C₃₋₁₅ cycloalkyl), —NH(heterocyclyl), —NH(C₆₋₁₀ aryl), —NH(5-10 membered heteroaryl), —N(cycloalkyl)₂, —N(C₁₋₉ alkyl)₂, —N(C₁₋₈ haloalkyl)₂, —N(C₂₋₆ alkenyl)₂, —N(C₂₋₆ alkynyl)₂, —N(C₃₋₁₅ cycloalkyl)₂, —N(heterocyclyl)₂, —N(C₆₋₁₀ aryl)₂, —N(5-10 membered heteroaryl)₂, —N(C₁₋₉ alkyl)(C₁.s haloalkyl), —N(C₁₋₉ alkyl)(C₂₋₆ alkenyl), —N(C₁₋₉ alkyl)(C₂₋₆ alkynyl), —N(C₁₋₉ alkyl)(C₃₋₁₅ cycloalkyl), —N(C₁₋₉ alkyl)(heterocyclyl), —N(C₁₋₉ alkyl)(C₆₋₁₀ aryl), —N(C₁₋₉ alkyl)(5-10 membered heteroaryl), —O(C₁₋₉ alkyl), —O(C₁₋₈ haloalkyl), —O(C₂₋₆ alkenyl), —O(C₂₋₆ alkynyl), —O(C₃₋₁₅ cycloalkyl), —O(heterocyclyl);

wherein R₅ is optionally substituted with one or more substituents each independently selected from the group consisting of halo, C₁₋₉ alkyl, C₁₋₈ haloalkyl, C₁₋₆ alkoxy, C₁₋₆ haloalkoxy, C₃₋₁₅ cycloalkyl, heterocyclyl, C₆₋₁₀ aryl, 5-10 membered heteroaryl, wherein the C₁₋₉ alkyl and C₃₋₁₅ cycloalkyl are optionally substituted with one or more halo.

In some embodiments, wherein R₅ is independently selected from the group consisting of H, halogen, C₁₋₉ alkyl, C₁₋₈ haloalkyl, C₁₋₆ alkoxy, C₁₋₆ haloalkoxy, C₂₋₆ alkoxyalkyl, C₃₋₁₅ cycloalkyl, heterocyclyl, C₆₋₁₀ aryl, 5-10 membered heteroaryl, —NH(C₁₋₉ alkyl), —NH(C₁₋₈ haloalkyl), —NH(C₃₋₁₅ cycloalkyl), —NH(heterocyclyl), —N(cycloalkyl)₂, —N(C₁₋₉ alkyl)₂, —N(C₁₋₈ haloalkyl)₂, —N(C₃₋₁₅ cycloalkyl)₂, —N(C₁₋₉ alkyl)(C₁₋₈ haloalkyl), —N(C₁₋₉ alkyl)(C₃₋₁₅ cycloalkyl), —N(C₁₋₉ alkyl)(heterocyclyl), —N(C₁₋₉ alkyl)(C₆₋₁₀ aryl), —N(C₁₋₉ alkyl)(5-10 membered heteroaryl), —O(C₁₋₉ alkyl), —O(C₁₋₈ haloalkyl), —O(C₃₋₁₅ cycloalkyl), —O(heterocyclyl);

wherein R₅ is optionally substituted with one or more substituents each independently selected from the group consisting of halo, C₁₋₉ alkyl, C₁₋₈ haloalkyl, C₁₋₆ alkoxy, C₁₋₆ haloalkoxy, C₃₋₁₅ cycloalkyl, heterocyclyl, C₆₋₁₀ aryl, 5-10 membered heteroaryl, wherein the C₁₋₉ alkyl and C₃₋₁₅ cycloalkyl are optionally substituted with one or more halo.

In some embodiments, the compound of the present application is selected from the group consisting of the following compounds:

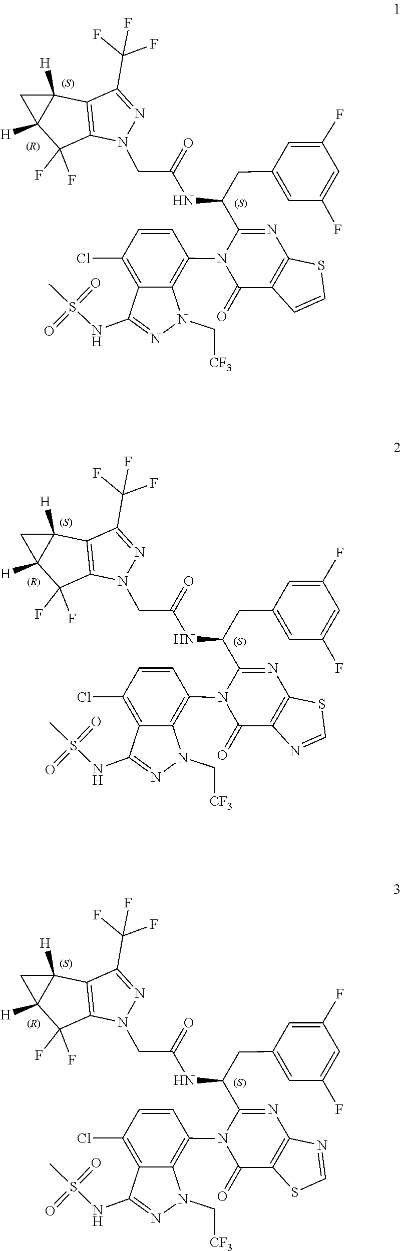

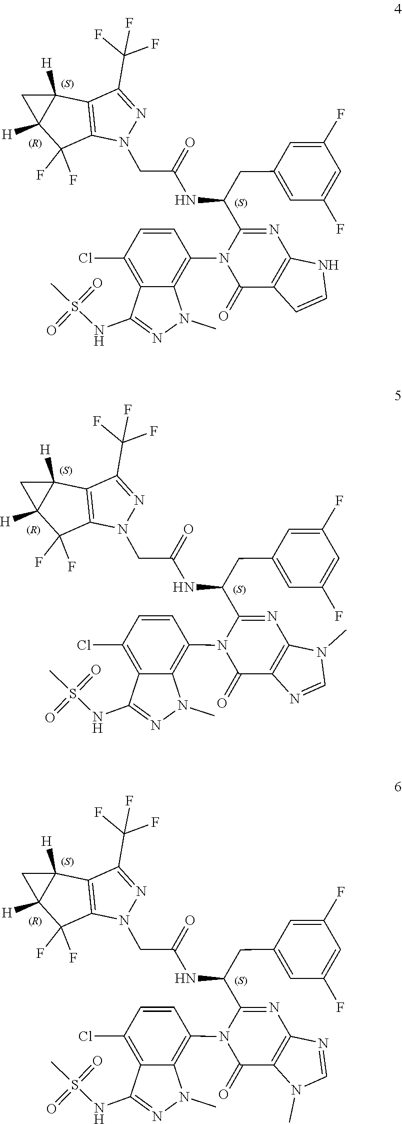

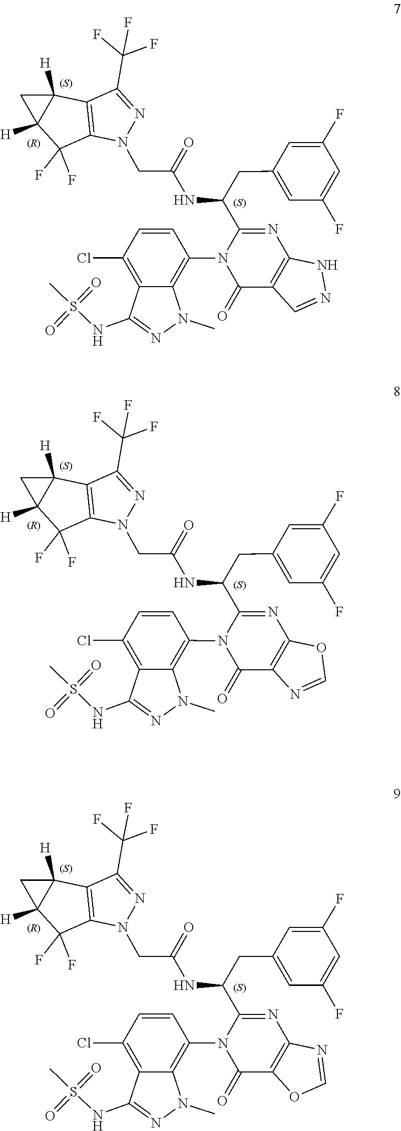

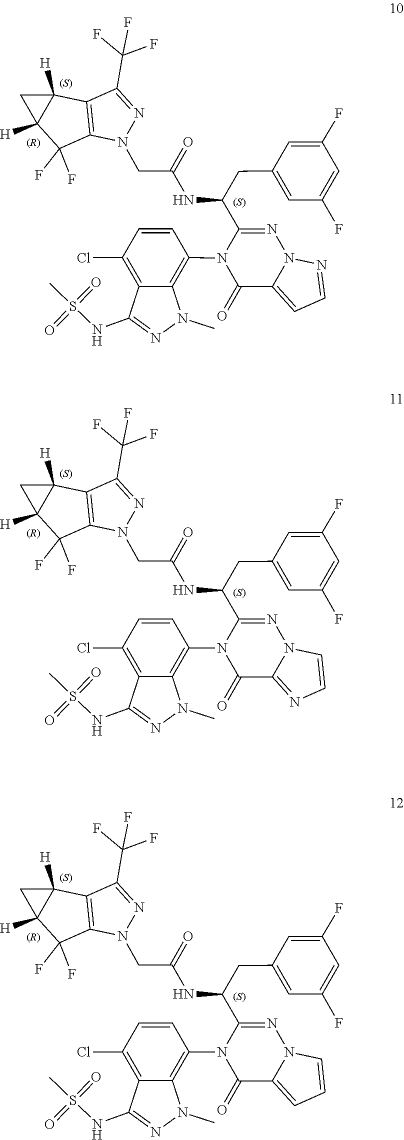

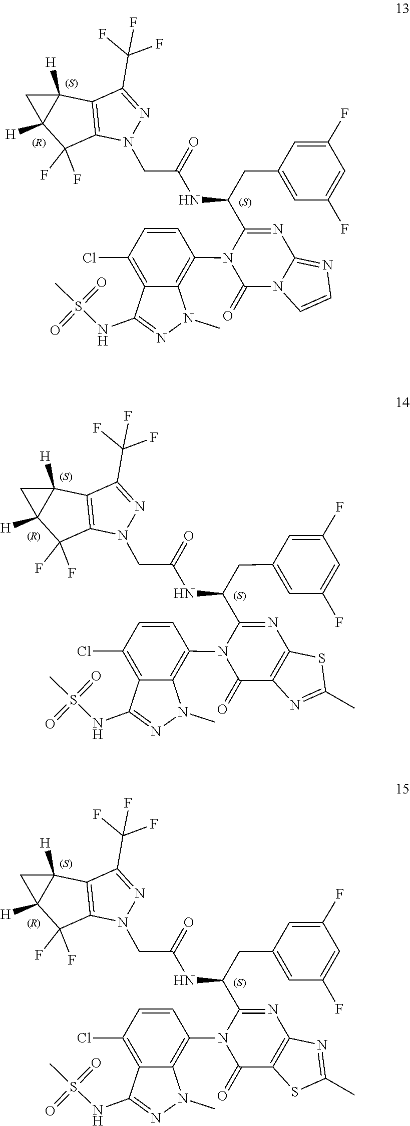

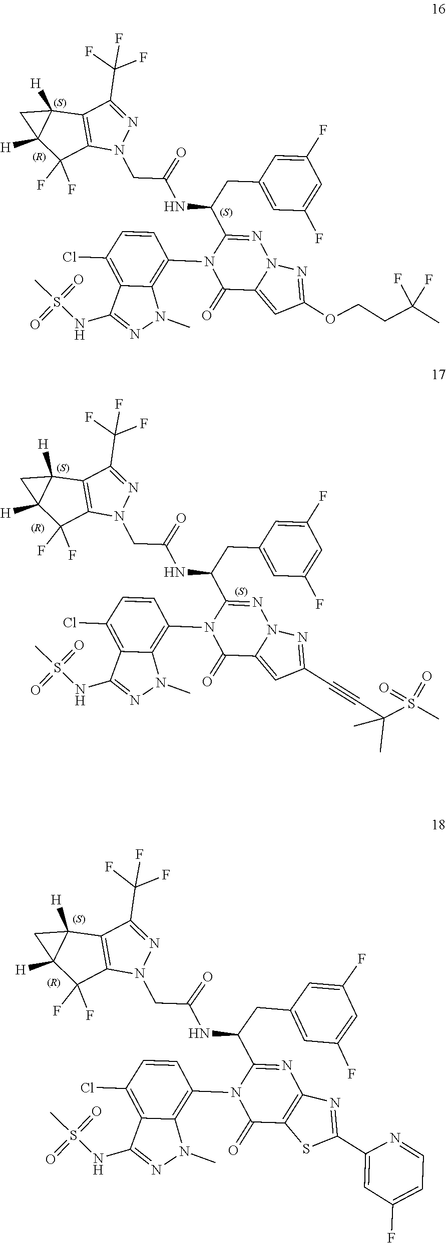

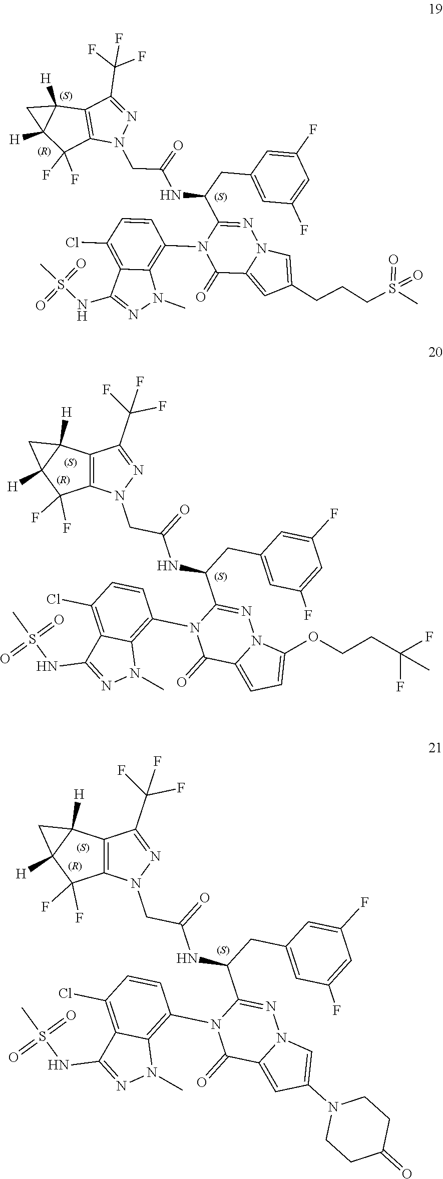

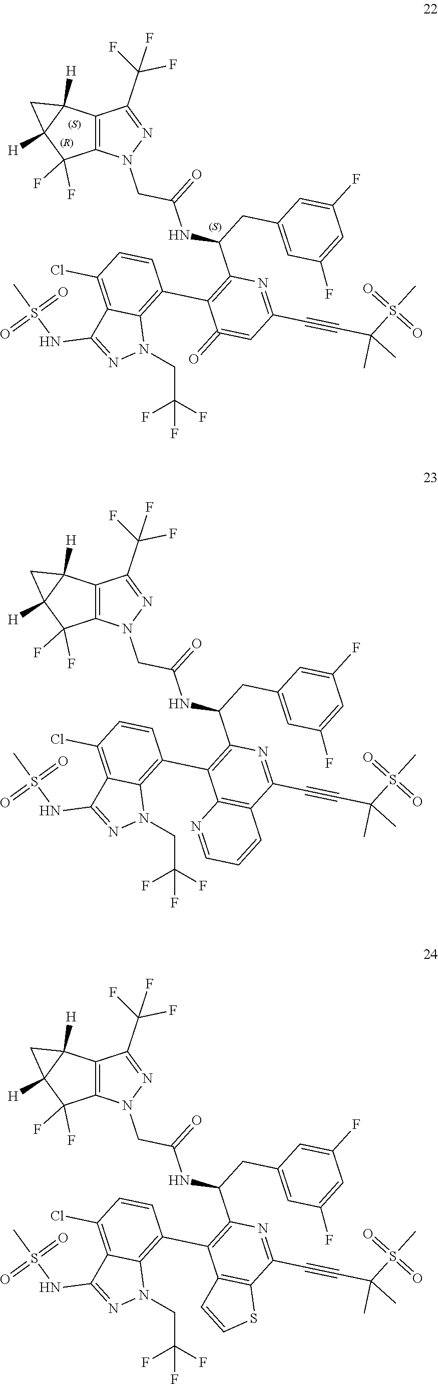

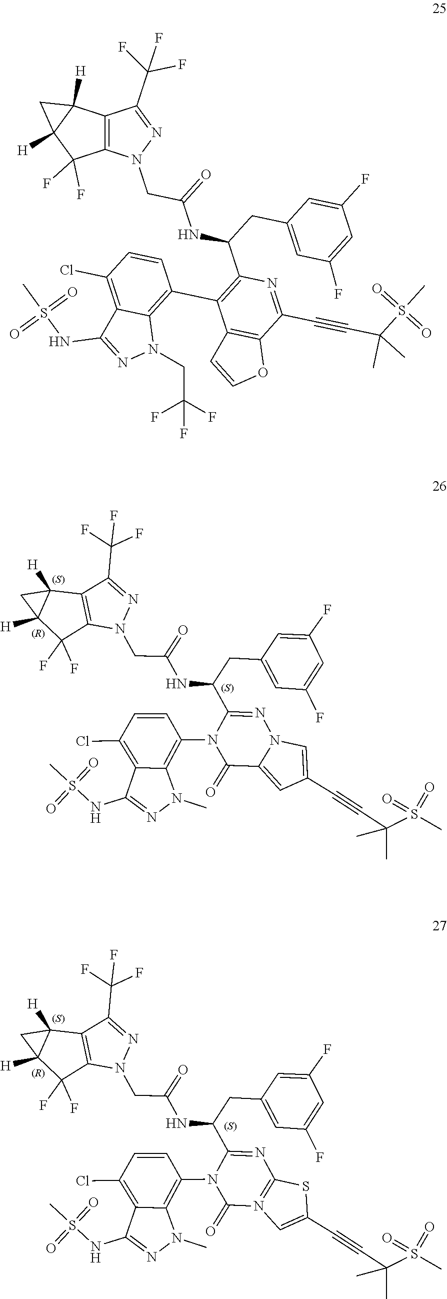

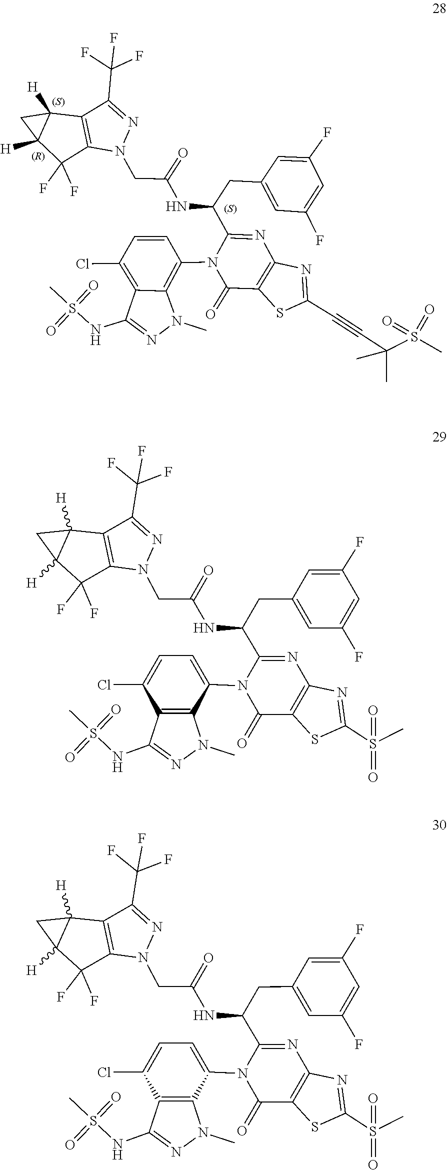

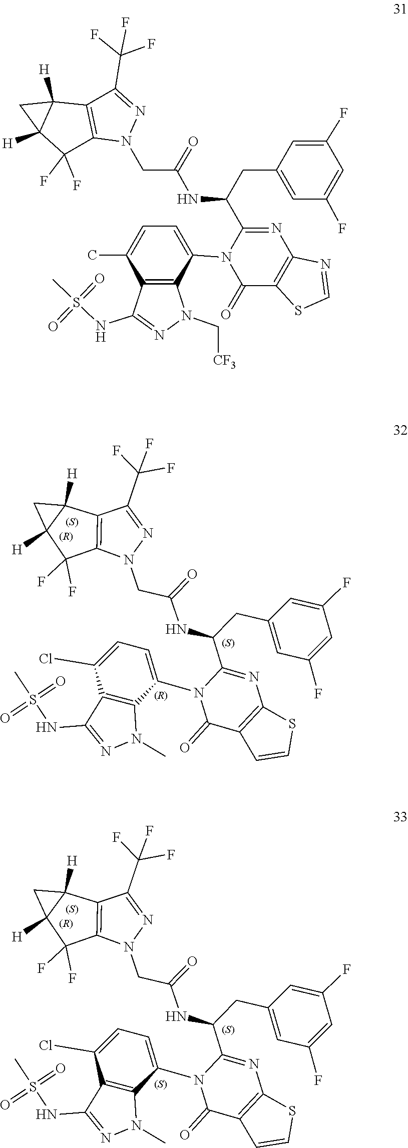

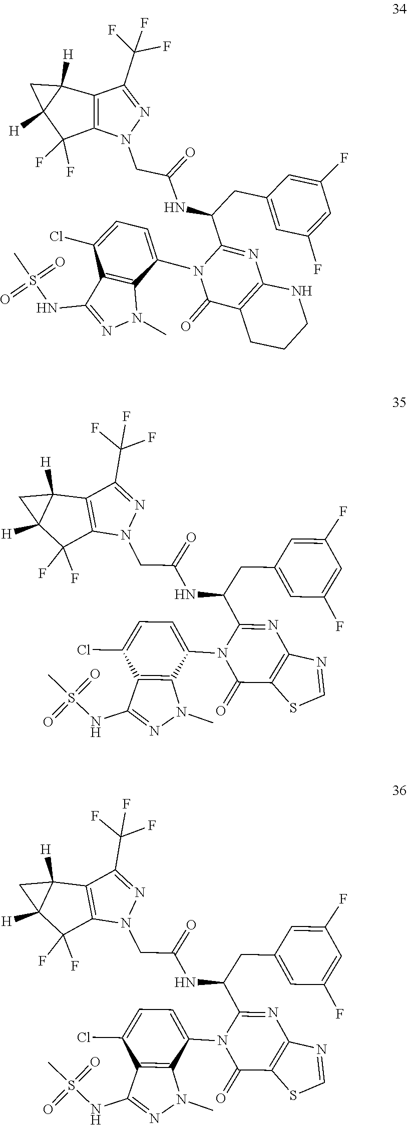

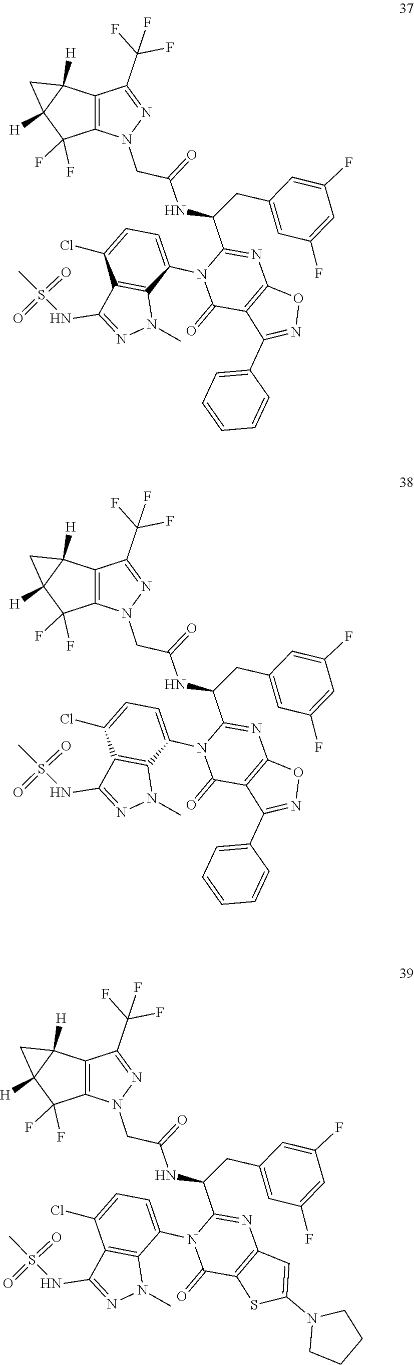

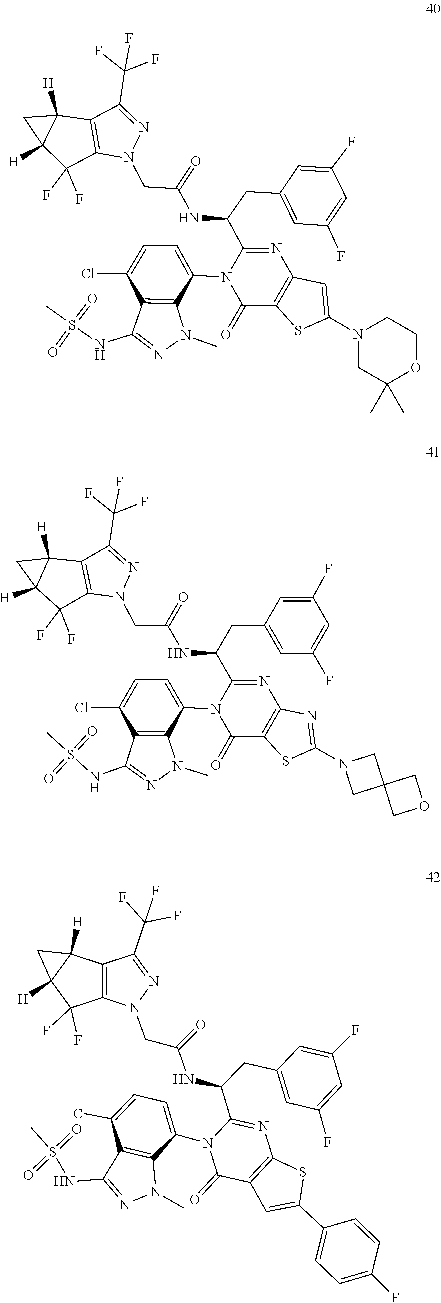

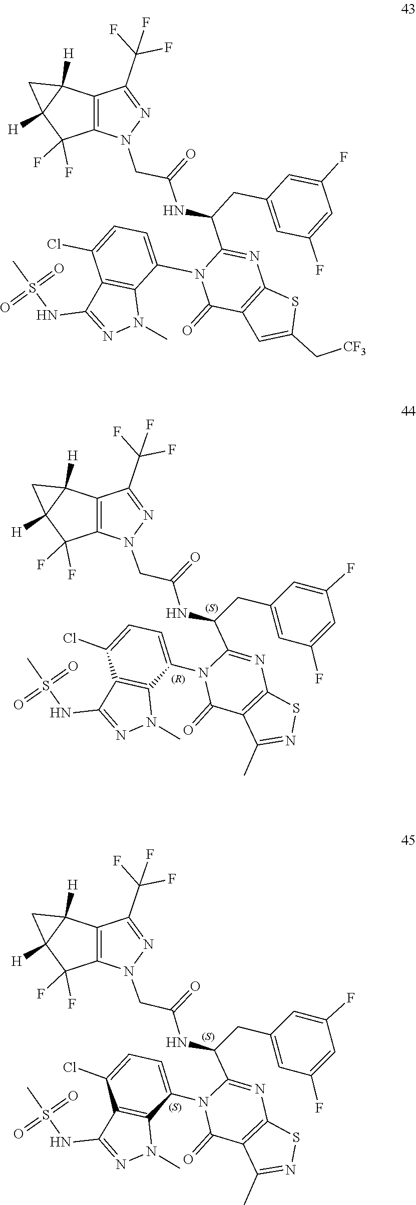

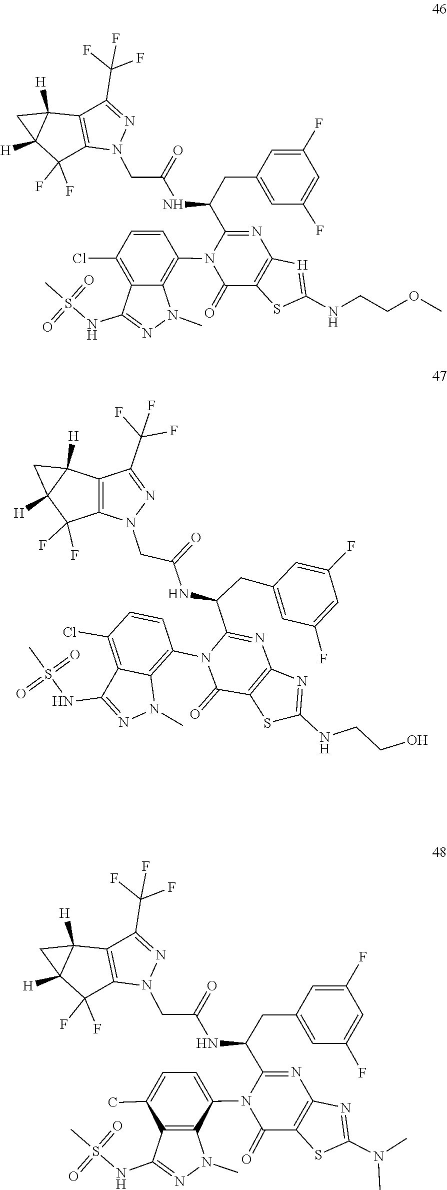

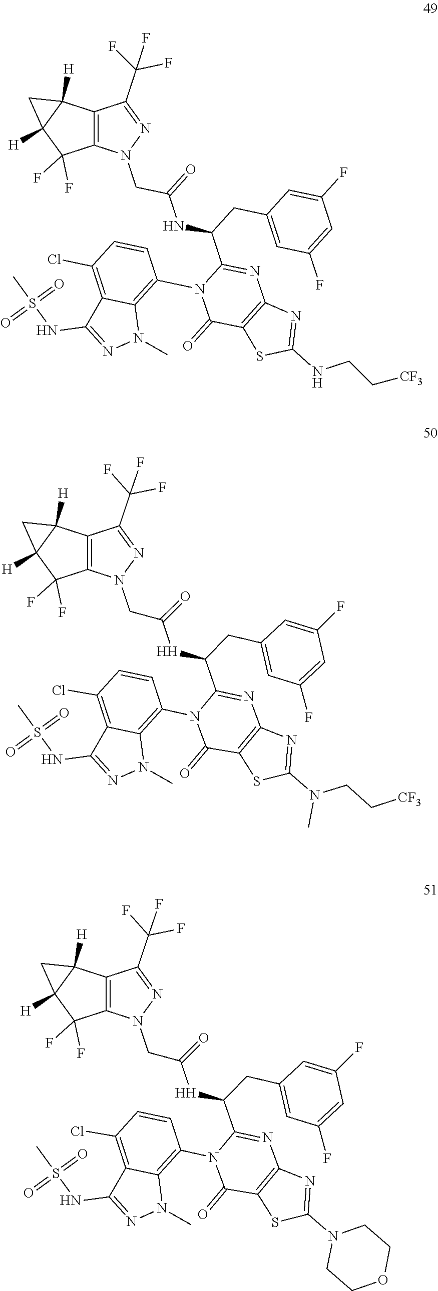

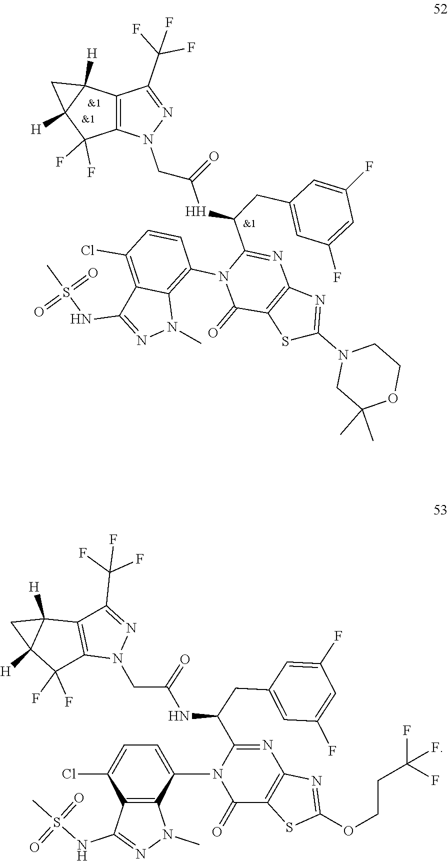

### III. Methods of Treatment

In accordance with another aspect of the instant application, methods for treating, inhibiting, and/or preventing a disease or disorder in a subject in need thereof are provided. The methods comprise administering to the subject at least one compound of the formula (I), optionally within a composition comprising a pharmaceutically acceptable carrier. In a particular embodiment, the composition is formulated for oral administration, intramuscular injection, or subcutaneous injection. In a particular embodiment, the disease or disorder is viral infection. In a particular embodiment, the viral infection is an HIV. In some embodiments, the method comprises the step of administering to a subject in need of such treatment an effective amount of a compound of Formula I, or a stereoisomer thereof, or a pharmaceutically acceptable salt thereof, or a deuterated compound thereof, or the pharmaceutical composition. In some embodiments, the method further comprises the step of administering to the subject of at least one other therapeutic agent. In some embodiments, the at least one other therapeutic agent is selected from the group consisting of dolutegravir, bictegravir, lamivudine, fostemsavir, cabotegravir, maraviroc, rilpiverine, atazanavir, tenofovir alafenamide, islatravir, doravirine, and darunavir.

### EXAMPLES

### Synthesis

The compounds of the disclosure may be prepared using methods disclosed herein and routine modifications thereof which will be apparent given the disclosure herein and methods well known in the art. Conventional and well-known synthetic methods may be used in addition to the teachings herein. The synthesis of typical compounds of Formula I, or a pharmaceutically acceptable salt thereof, e.g., compounds having structures described by one or more of Formula I, or other formulas or compounds disclosed herein, may be accomplished as described in the following examples.

### General Syntheses

Typical embodiments of compounds in accordance with the present application may be synthesized using the general reaction schemes and/or examples described below. It will be apparent given the description herein that the general schemes may be altered by substitution of the starting materials with other materials having similar structures to result in products that are correspondingly different. Descriptions of syntheses follow to provide numerous examples of how the starting materials may vary to provide corresponding products. Starting materials are typically obtained from commercial sources or synthesized using published methods for synthesizing compounds which are embodiments of the present application, inspection of the structure of the compound to be synthesized will provide the identity of each substituent group The identity of the final product will generally render apparent the identity of the necessary starting materials by a simple process of inspection, given the examples herein. Group labels (e.g., R1, R2) used in the reaction schemes herein are for illustrative purposes only and unless otherwise specified do not necessarily match by name or function the labels used elsewhere to describe compounds of Formula I or aspects or fragments thereof.

### Synthetic Reaction Parameters

The compounds of this disclosure can be prepared from readily available starting materials using, for example, the following general methods and procedures. It will be appreciated that where typical or preferred process conditions (i.e., reaction temperatures, times, mole ratios of reactants, solvents, pressures, etc.) are given; other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts (1999) Protecting Groups in Organic Synthesis, 3rd Edition, Wiley, New York, and references cited therein.

Furthermore, the compounds of the present application may contain one or more chiral centers. Accordingly, if desired, such compounds can be prepared or isolated as pure stereoisomers, i.e., as individual enantiomers or diastereomers or as stereoisomer-enriched mixtures. All such stereoisomers (and enriched mixtures) are included within the scope of this disclosure, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents, and the like.

The starting materials for the following reactions are generally known compounds or can be prepared by known procedures or obvious modifications thereof. For example, many of the starting materials are available from commercial suppliers such as Aldrich Chemical Co. (Milwaukee, Wisconsin, USA). Others may be prepared by procedures or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplemental (Elsevier Science Publishers, 1989) organic Reactions, Volumes 1-40 (John Wiley, and Sons, 1991), March's Advanced Organic Chemistry, (John Wiley, and Sons, 5th Edition, 2001), and Larock's Comprehensive Organic Transformations (VCH Publishers Inc., 1989).

The terms “solvent,” “inert organic solvent” or “inert solvent” refer to a solvent inert under the conditions of the reaction being described in conjunction therewith (including, for example, benzene, toluene, acetonitrile, tetrahydrofuran (“THF”), N, N-dimethylformamide (“DMF”), chloroform, methylene chloride (or dichloromethane), diethyl ether, methanol, pyridine and the like). Unless specified to the contrary, the solvents used in the reactions of the present application are inert organic solvents, and the reactions are carried out under an inert gas, preferably nitrogen.

The term “q.s.” means adding a quantity sufficient to achieve a stated function, e.g., to bring a solution to the desired volume (i.e., 100%).

Compounds as provided herein may be synthesized according to the general schemes provided below. In the Schemes below, it should be appreciated that each of the compounds shown therein may have protecting groups as required present at any step. Standard protecting groups are well within the pervue of one skilled in the art.

### Example 1: Preparation of Intermediate

### (1) Preparation of N-(7-amino-4-chloro-1-methyl-1H-indazol-3-yl)-N-(4-methoxybenzyl)methanesulfonamide (Int A)

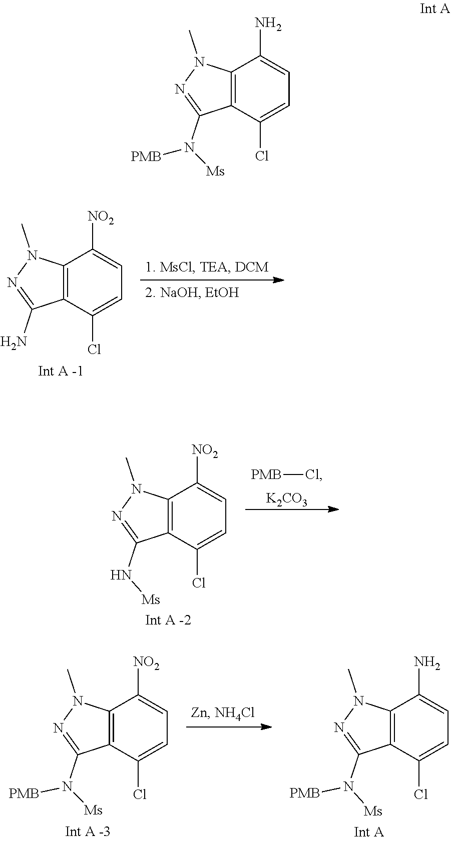

Trimethylamine (TEA) (2.68 g, 26.48 mmol) was added to a solution of Int A-1 (1.0 g, 4.41 mmol, CAS: 1192263-90-3) in DCM (10 ml) at 0-5° C. The solution of methanesulfonyl chloride (MsCl) (2.0 g, 17.6 mmol) of DCM (5 ml) was added dropwise into the mixture solution, and stirred for 2 hours. Then water (15 ml) was added, and stirred for 10 mins. Then the organic layers were separated and concentrated under vacuum to afford a solid. The solid was dissolved into ethanol (10 ml), followed 5% NaOH solution (10 ml), and stirred for 3 hours at room temperature. pH was adjusted to 4-5, and solids was precipitated. After filtration, the Int A-2 was obtained, 1.54 g, yield: 91.2%. LCMS: [M+H]⁺: 305.0, 307.0.

PMB-Cl (1.19 g, 7.6 mmol) and K₂CO₃ (1.40 g, 10.11 mol) were added to a solution of Int A-2 (1.54 g, 5.05 mmol) in DMF (15 ml) at 80-90° C. and stirred for 4 hours. After cooling, the mixture solution was added into an ice water (20 ml) and stirred for 30 mins. The Int A-3 was obtained by filtration with the mixture solution, 1.89 g, yield: 88.0%. LCMS: [M+H]⁺: 425.1, 427.1.

Zn (1.16 g, 17.8 mmol) and NH₄Cl (1.9 g, 35.6 mmol) were added to a solution of Int A-3 (1.89 g, 4.45 mmol) in ethanol (15 ml) and water (5 ml) and stirred for 2 hours at 80-90° C. After cooling, the mixture was concentrated under vacuum to afford a crude solid. The solid was dissolved into the ethyl acetate (20 ml) and washed the water (20 ml×2), and the organic layer was concentrated under vacuum to afford Int A, 1.43 g, yield:81.4%. LCMS: [M+H]⁺: 395.1, 397.1.

### (2) Preparation of tert-butyl ((1S)-1-(6-bromo-3-(4-chloro-3-(N-(4-methoxybenzyl)methylsulfonamido)-1-methyl-1H-indazol-7-yl)-4-oxo-3,4-dihydropyrazolo[5,1-f][1,2,4]triazin-2-yl)-2-(3,5-difluorophenyl)ethyl)carbamate (Int B)

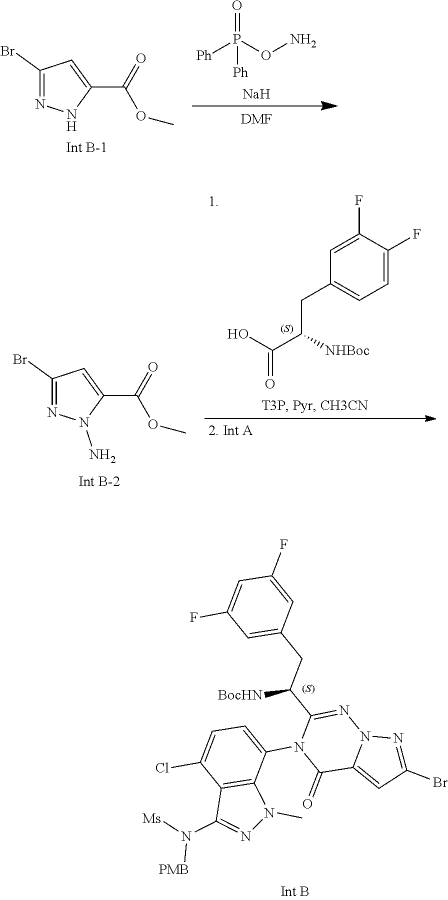

NaH (1.2 g, 29.3 mmol, 1.2eq.) (60% in oil) was added to a solution of Int B-1 (5.0 g, 24.4 mmol, 1 eq) in DMF (50 ml) at 0˜5° C. and stirred for 10 mins. Then (aminooxy)diphenylphosphine oxide (6.83 g, 29.3 mmol, 1.2 eq) was added to the mixture at 0° C., and the reaction was stirred for 15 h at 25° C. The mixture was poured into water (50 ml) and extracted with EtOAc (50 ml×2). The layers were concentrated under reduce pressure. The residue was purified by silica gel column chromatography (PE:EA=1:0-10:1) to afford Int B-2, 4.93 g. yield: 91.8%. LCMS: [M+H]⁺: 220.0.

(S)-2-((tert-butoxycarbonyl)amino)-3-(3,5-difluorophenyl)propanoic acid (2.74 g, 9.1) and pyridine (3.6 g, 45.5 mmol) were added to a solution of Int B-2 (2.0 g, 9.1 mmol) in CH₃CN(20 ml) at 0˜5° C., followed addition dropwise T3P (14.46 g, 45.45 mmol) and stirred for 3 hours. Then the Int A (3.59 g, 9.1 mmol) was added and stirred for 6 hours at 80˜85° C. After cooling, the mixture was concentrated to get a oil, which was purification by silica gel column chromatography, eluted with DCM/MEOH (10:1). The Int B was afford, 4.6 g. yield: 59.7%. LCMS: [M+H]⁺: 847.1.

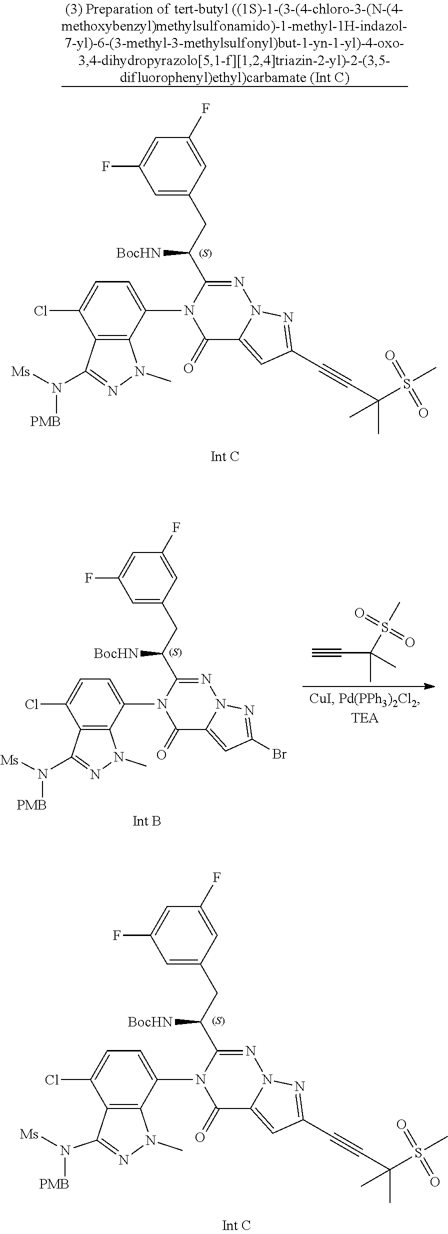

3-methyl-3-(methylsulfonyl)but-1-yne (103 mg, 0.71 mmol), CuI (13.5 mg, 0.07 mmol), Pd(PPh₃)₂Cl₂ (25 mg, 0.036 mmol) and TEA (215, 1 mg 2.13 mmol) were added to a solution of Int B(0.6 g, 0.71 mmol) in DMF (5 ml). At a N₂ atmosphere, the mixture was heated to 60-65° C. and stirred for 6 hours. After cooling, water (5 ml) was added and stirred for 15 mins, then filtrated to get a solid. After purification by silica gel column chromatography, eluted with DCM/MEOH (10:1), the Int C was obtained, 0.43 g, yield:66.5%. LCMS: [M+H]⁺: 913.2.

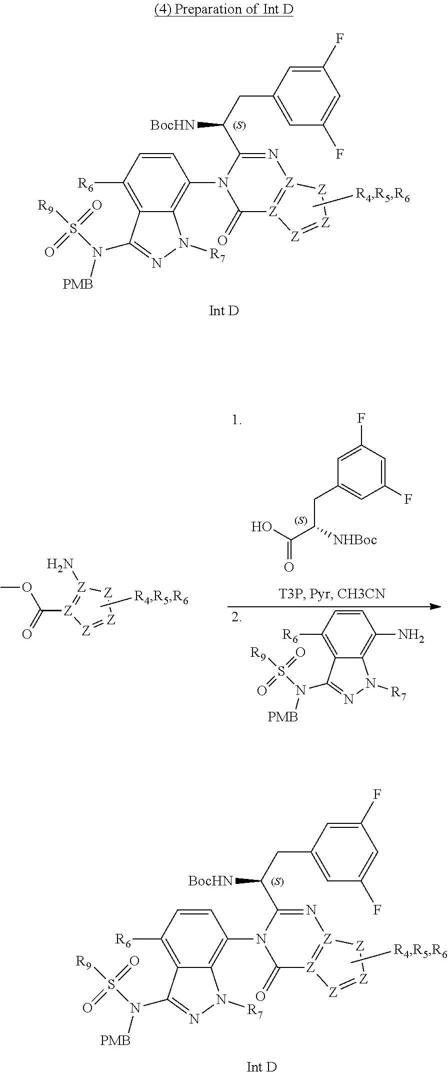

Int D is a common intermediate, which can be prepared the method of Int B using a suitable reagent. The Int D include the structures as followed:

TABLE 1
The list of Int D

| | | (m/z) |
|---|---|---|
| Int D | Structure | [M + H]⁺ |
| Int D-1 | 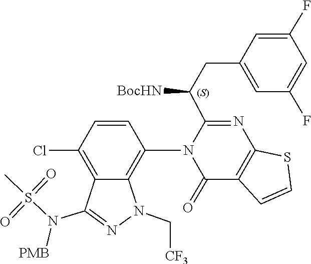 | 852.2 |
| Int D-2 | 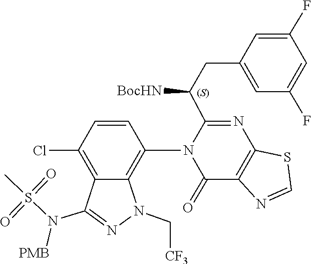 | 853.2 |
| Int D-3 | 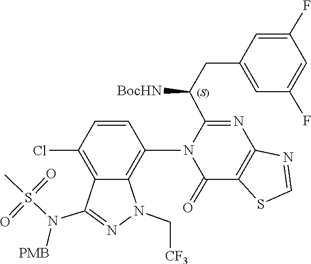 | 853.2 |
| Int D-4 | 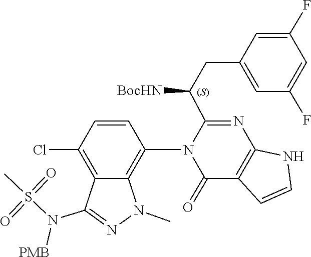 | 768.2 |
| Int D-5 | 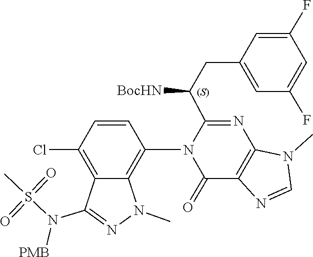 | 783.2 |
| Int D-6 | 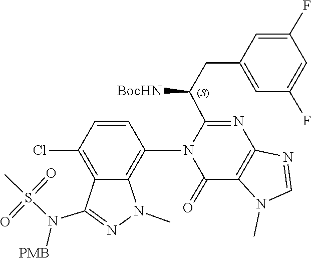 | 783.2 |
| Int D-7 | 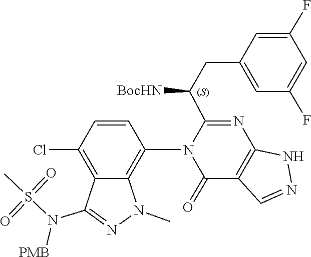 | 769.2 |
| Int D-8 | 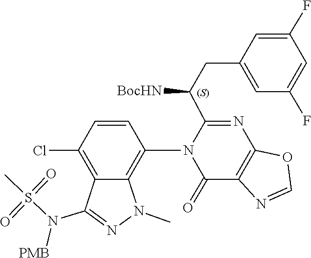 | 770.2 |
| Int D-9 | 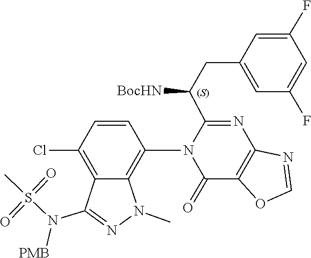 | 770.2 |
| Int D-10 | 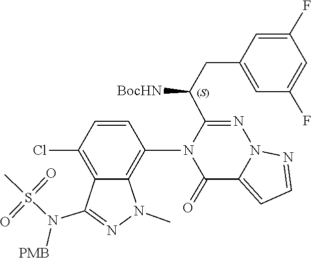 | 769.2 |
| Int D-11 | 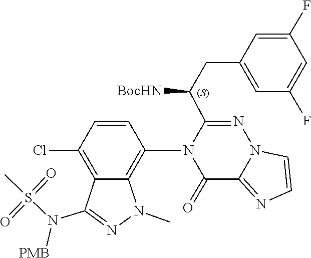 | 769.2 |
| Int D-12 | 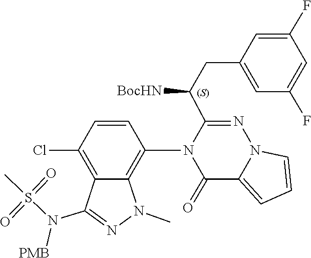 | 768.2 |
| Int D-13 | 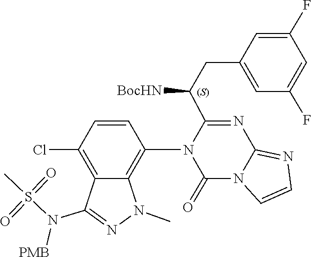 | 769.2 |
| Int D-14 | 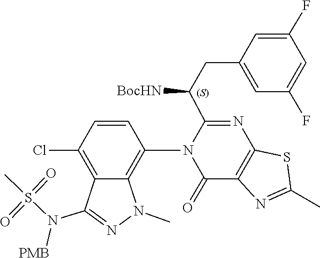 | 800.2 |
| Int D-15 | 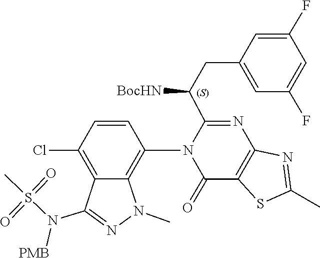 | 800.2 |
| Int D-16 | 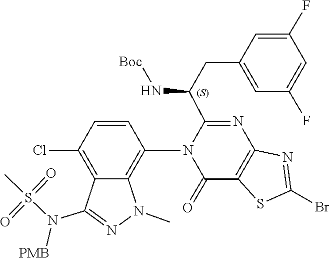 | 864.1 |

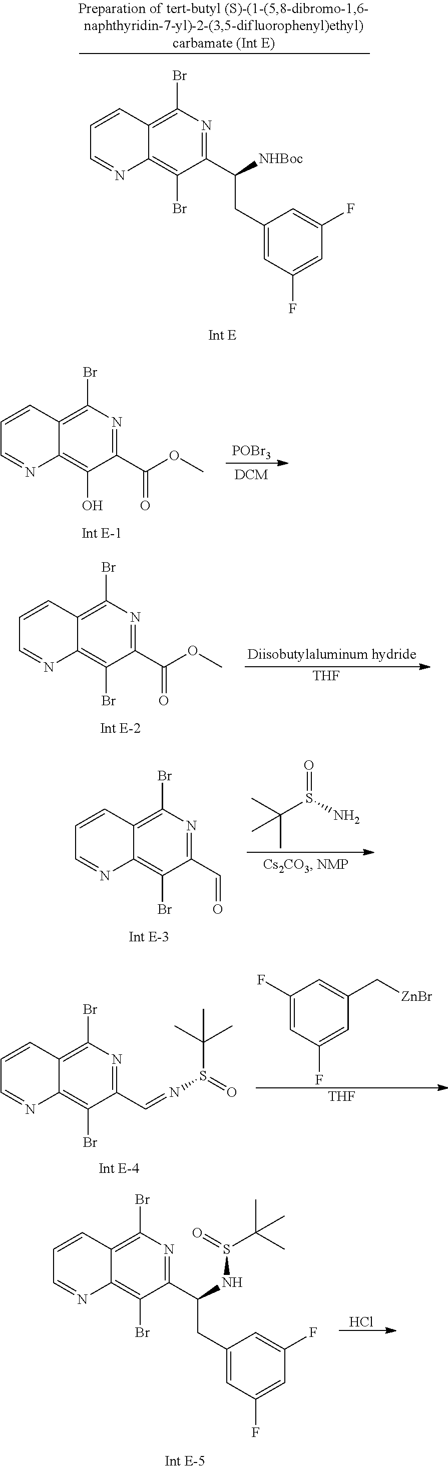

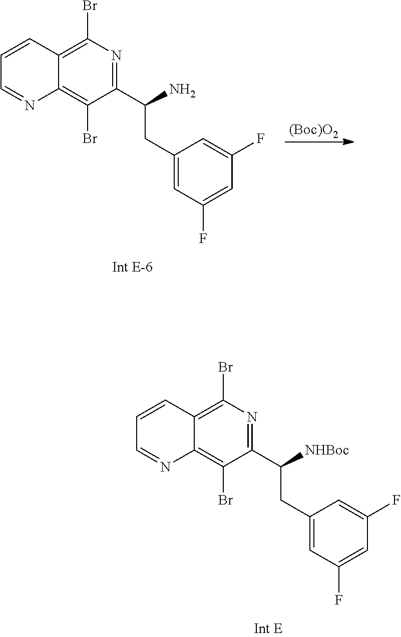 [SMILES: "(Boc)O2" ; N[C@@H](Cc1cc(F)cc(F)c1)c1nc(Br)c2cccnc2c1Br ; CC(C)(C)OC(=O)N[C@@H](Cc1cc(F)cc(F)c1)c1nc(Br)c2cccnc2c1Br]

The POBr₃ (1.32 g, 4.6 mmol) was added into the solution of Int E-1 (1.0 g, 3.5 mmol) in DCM (10 ml) dropwise at −5˜0° C., and stirred for 3 hours. The mixture was added slowly into an ice saturated sodium hydrogen carbonate solution, and a solid was precipitated. After filtration, wash by water and dry, Int E-2 was obtained, 1.15 g, yield:94.1%. LCMS: [M+H]⁺:344.9.

The diisobutylaluminum hydride in THF (10M in THF, 3.3 ml) was added into the solution of Int E-2 (1.1 g, 3.32 mmol) in THF (10 ml) dropwise at −78° C., and stirred for 1 hour. Then the mixture was warmed naturally to room temperature, and saturated ammonium chloride solution was added. EtOAc (20 ml) was added and extracted to get an organic layer. The organic layer was concentrated under reduce pressure and purified by silica gel column chromatography to afford Int E-3, 0.87 g, yield: 82.8%. LCMS: [M+H]⁺:314.9.

The solution of Int E-3 (0.8 g, 2.53 mmol) in NMP (N-methyl pyrrolidone) (10 ml) was added (S)-2-methylpropane-2-sulfinamide (0.338 g, 2.79 mmol) at room temperature. The Cs₂CO₃ (1.24 g, 3.8 mmol) was added into the mixture and stirred for 2 hours. An ice water (20 ml) was added into the mixture, and a solid was precipitated. After filtration, wash by water and dry, Int E-4 was obtained, 0.98 g, yield:92.3%. LCMS: [M+H]⁺:417.9.

At a N₂ atmosphere, Int E-4 (0.95 g, 2.27 mmol) was dissolved into DMF (10 ml). After cooling the mixture to 0˜5° C., (3,5-difluorobenzyl)zinc(II) bromide (1M in THF, 3.41 ml) was added dropwise and stirred for 3 hours. To the reaction mixture, 5% AcOH in water (30 ml) was added over 10 mins followed by Cyclopentyl Methyl ether. The mixture was stirred for 5 mins, warmed to room temperature, and the organic layer was separated and washed with brine. Then the layer was concentrated under reduce pressure and purified by silica gel column chromatography to afford Int E-5, 0.56 g, yield: 45.3%. LCMS: [M+H]⁺:546.0.

The solution of Int E-5 (0.5 g, 0.9 mmol) in THF (5 ml) was added HCl (37%, 0.5 ml) and stirred for 1 hour at room temperature. EtOAc (10 ml) and saturated sodium hydrogen carbonate solution (10 ml) were added and extracted to get an organic layer. The organic layer was concentrated under reduce pressure to get a crude Int E-6 without purification and used in the next reaction step. LCMS: [M+H]⁺:441.9 The solution of Int E-6 in DCM (5 ml) was added the (Boc)₂O (294 mg, 1.35 mmol) and DIPEA (174 mg, 1.35 mmol) at room temperature and stirred overnight. Water (5 ml) was added and stirred for 5 mins. The organic layer was separated. After concentration under reduce pressure and purification by silica gel column chromatography, the Int E was obtained, 0.42 g. LCMS: [M+H]⁺:544.2.

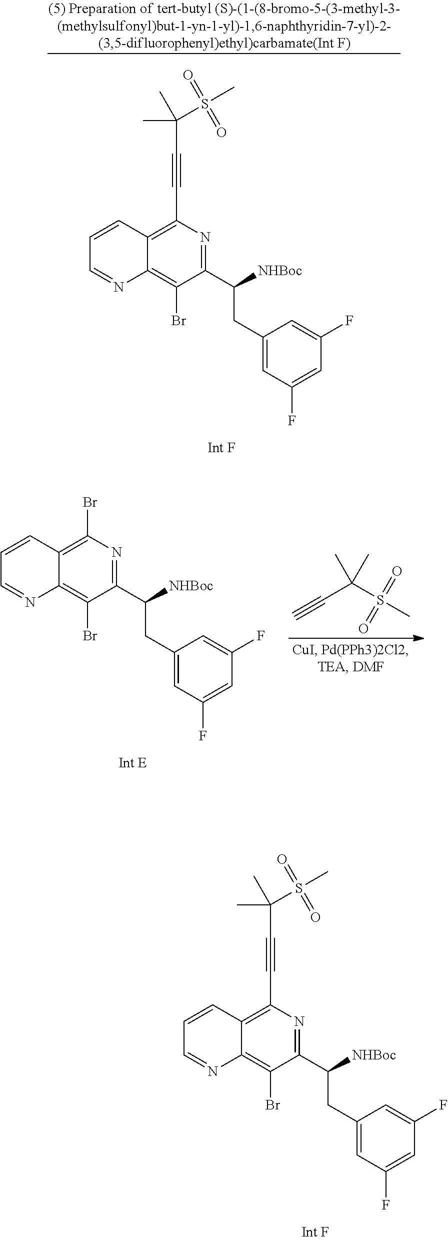

At a N₂ atmosphere, Int E (0.4 g, 0.74 mmol), 3-methyl-3-(methylsulfonyl)but-1-yne (140 mg, 0.96 mmol), TEA (149 mmol, 1.48 mmol), Pd(PPh₃)₂Cl₂(26 mg, 0.037 mmol) and CuI (7.0 mg, 0.037 mmol) were dissolved into DMF (5 ml), and stirred for 2 hours at 50˜60° C. Water (10 ml) and EtOAc (10 ml) were added into the mixture, and stirred for 10 mins. The organic layer was separated and washed with brine. After concentration under reduce pressure and purification by silica gel column chromatography, the Int F was obtained, 0.24 g, yield: 53.6%. LCMS: [M+H]⁺:608.1.

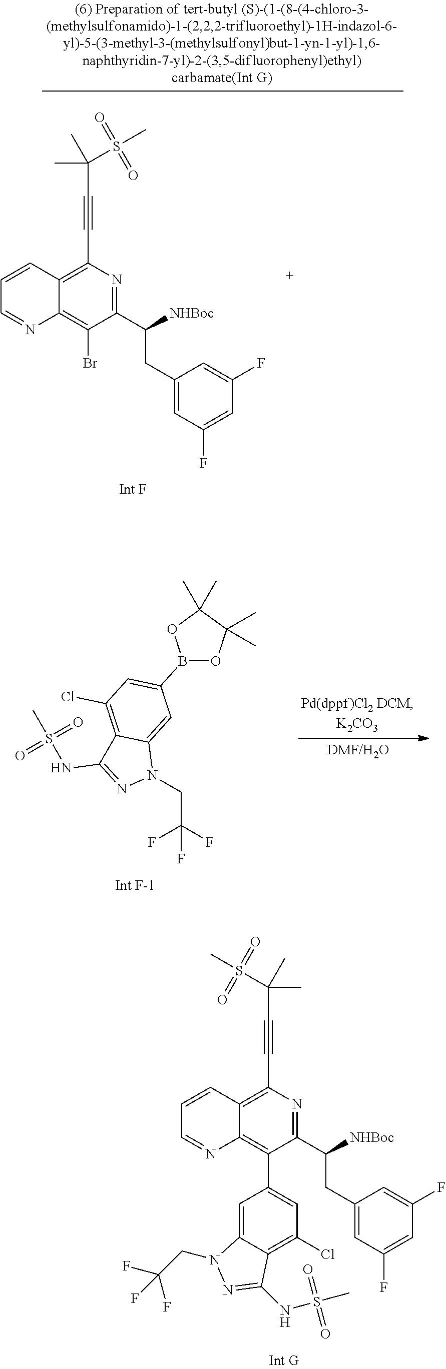 [SMILES: CC(C)(C)OC(=O)N[C@@H](Cc1cc(F)cc(F)c1)c1nc(C#CC(C)(C)S(C)(=O)=O)c2cccnc2c1Br ; CC1(C)OB(c2cc(Cl)c3c(NS(C)(=O)=O)nn(CC(F)(F)F)c3c2)OC1(C)C ; CC(C)(C)OC(=O)N[C@@H](Cc1cc(F)cc(F)c1)c1nc(C#CC(C)(C)S(C)(=O)=O)c2cccnc2c1-c1cc(Cl)c2c(NS(C)(=O)=O)nn(CC(F)(F)F)c2c1]

At a N₂ atmosphere, Int F (0.15 g, 0.25 mmol), Int F-1 (0.17 g, 0.26 mmol), Pd(dppf)Cl₂·DCM (24.3 mg, 0.03 mmol) and K₂CO₃ (69 mg, 0.5 mmol) in DMF/H₂O (4 ml/1 ml) and stirred for 2 hours at 70˜75° C. After cooling, EtOAc (5 ml×2) and H₂O (5 ml) were added and stirred for 10 mins. The organic layer was separated and washed with brine. After concentration under reduce pressure and purification by silica gel column chromatography, the Int G was obtained, 0.18 g, yield: 85.4%. LCMS: [M+H]⁺: 855.2.

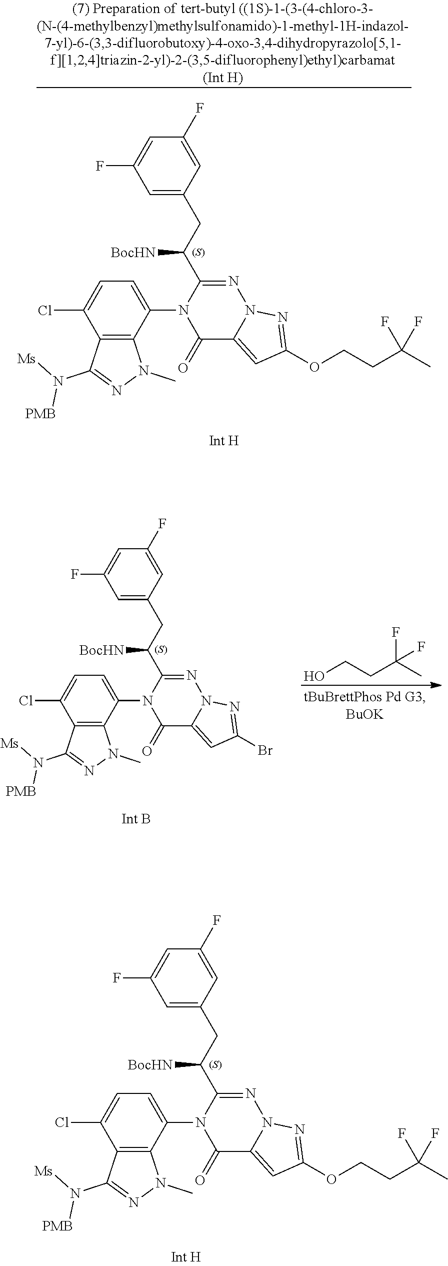

At a N₂ atmosphere, Int B (200 mg, 0.24 mmol), 3,3-difluorobutan-1-ol (39.0 mg, 0.35 mmol), tBuBrettPhos Pd G3 (21 mg, 0.024 mmol) and BuOK (53 mg, 0.48 mmol) were added into DMF/H₂O (5 ml/1 ml) and stirred for 16 hours at 70-75° C. After cooling, EtOAc (10 ml) and H₂O (10 ml) were added and stirred for 10 mins. The organic layer was separated and washed with brine twice. After concentration under reduce pressure and purification by preparative liquid phase chromatography (ACN:0.5% TFA, SunFire^{@} Prep C18 OBD™, 5 μM×19 mm×150 mm), the Int H was obtained, 35 mg, yield: 16.9%. LCMS: [M+H]⁺: 877.2.

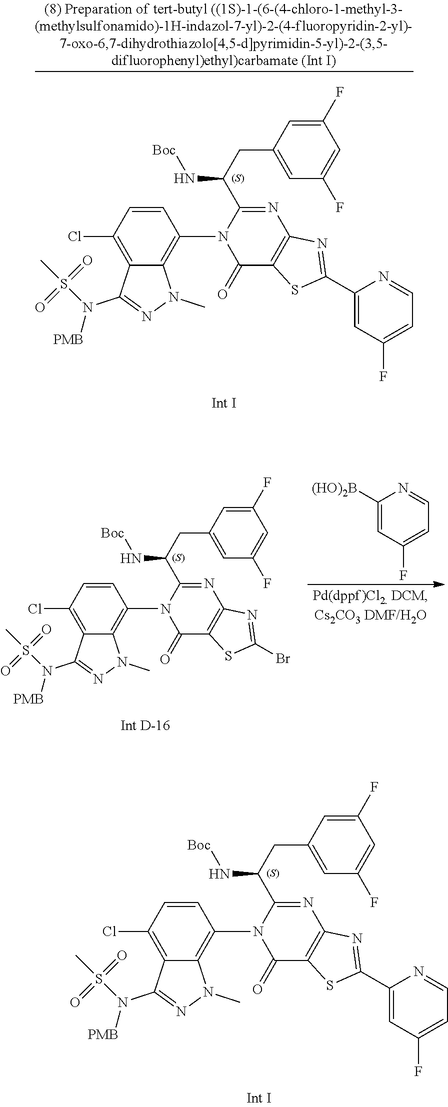

At a N₂ atmosphere, Int D-16 (120 mg, 0.14 mmol), (4-fluoropyridin-2-yl)boronic acid (23.8 mg, 0.15 mmol), Pd(dppf)Cl₂·DCM (11 mg, 0.014 mmol) and Cs₂CO₃ (68.5 mg, 0.21 mmol) were added into DMF/H₂O (5 ml/1 ml) and stirred for 2 hours at 70-75° C. After cooling, EtOAc (10 ml) and H₂O (10 ml) were added and stirred for 10 mins. The organic layer was separated and washed with brine twice. After concentration under reduce pressure and purification by preparative liquid phase chromatography (ACN: 0.5% TFA, SunFire^{@} Prep C18 OBD™, 5 μM×19 mm×150 mm), the Int I was obtained, 76 mg, yield: 62.2%. LCMS: [M+H]⁺: 761.1.

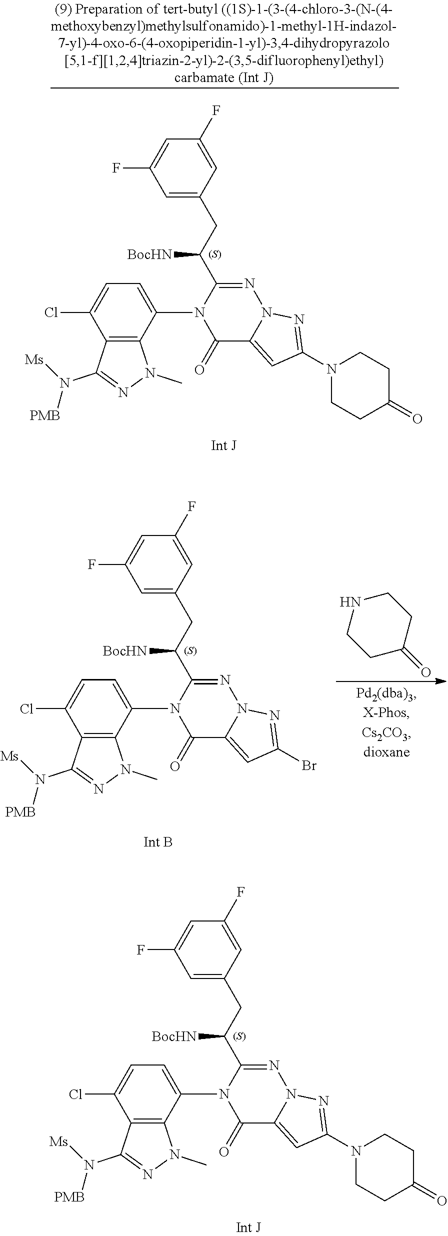

At a N₂ atmosphere, Int B (100 mg, 0.18 mmol), piperidin-4-one (14 mg, 0.14 mmol), Pd₂(dba)₃ (16.5 mg, 0.018 mmol), X-Phose (16.5 mg, 0.036 mmol) and Cs₂CO₃ (117 mg, 0.36 mmol) were added into dioxane (5 ml) and stirred for 2 hours at 100-110° C. After cooling, EtOAc (10 ml) and H₂O (10 ml) was added and stirred for 10 mins. The organic layer was separated and washed with brine twice. After concentration under reduce pressure and purification by preparative liquid phase chromatography (ACN: 0.5% TFA, SunFire^{@} Prep C18 OBD™, 5 μM×19 mm×150 mm), the Int J was obtained, 65 mg, yield: 63.6%. LCMS: [M+H]⁺: 865.3.

### Example 2: Preparation of Compounds

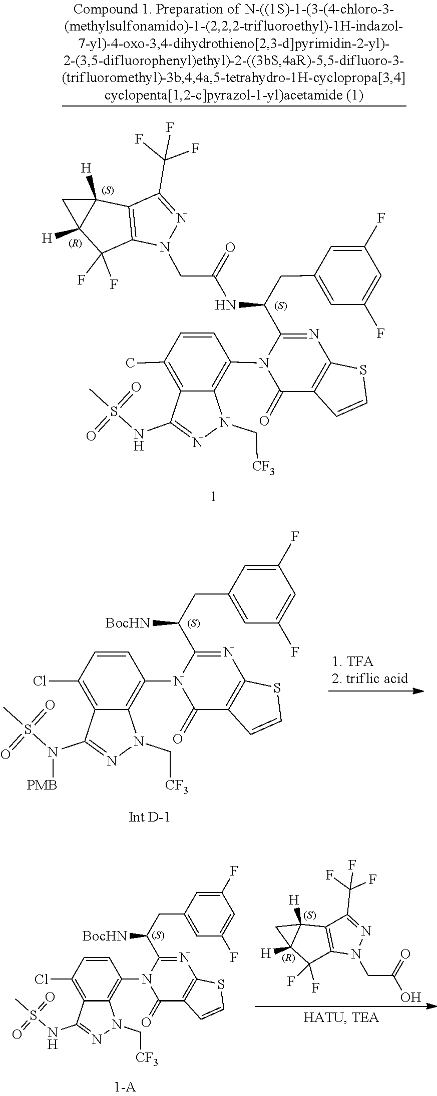

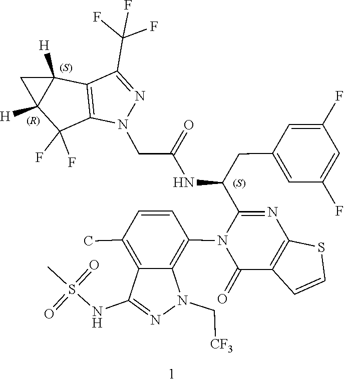

Trifluoroacetic acid (TFA) (1 ml) was added into the solution of Int D-1 (50 mg, 0.06 mmol) in DCM (2 ml) and stirred for 1 hour at room temperature. Then triflic acid (1 ml) was added into the mixture and keep stirring for 3 hours. The mixture was added slowly into an ice saturated sodium hydrogen carbonate solution (10 ml) followed the DCM (10 ml), and stirred for 10 mins. The organic layer was separated and washed with brine. Then the layer was concentrated under reduce pressure and purified by preparative liquid phase chromatography (ACN: 0.5% TFA, SunFire^{@} Prep C18 OBD™, 51 μM×19 mm×150 mm) to afford 1-A, 35 mg, yield: 94.4%. LCMS: [M+H]⁺:633.1.

Compound 1-A (35 mg, 0.055 mmol), 2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetic acid (15.6 mg, 0.055 mmol), HATU (31.4 mg, 0.083 mmol) and TEA (11.1 mg, 0.11 mmol) were added into DCM (5 ml), and stirred for 4 hours. H₂O (5 ml) was added into the mixture and stirred for 10 mins. The organic layer was separated and washed with brine. After concentration under reduce pressure and purification by preparative liquid phase chromatography (ACN: 0.5% TFA, SunFire^{@} Prep C18 OBD™, 5 μM×19 mm×150 mm), the title compound (1) was obtained, 21 mg. LCMS: [M+H]⁺:897.1.

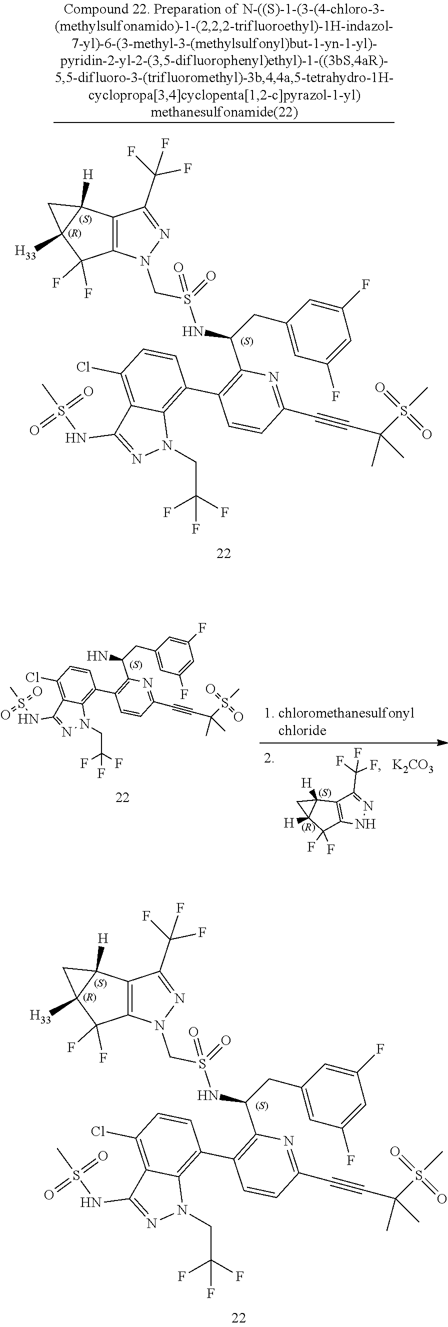

The chloromethanesulfonyl chloride (25.6 mg, 0.17 mmol) was added into the solution of compound 22-1 (110 mg, 0.16 mmol) and TEA (47.4 mg, 0.47 mmol) in DCM (10 ml) dropwise at 0˜5° C., and stirred for 1 hour. To the reaction mixture, H₂O (10 ml) was added, and stirred for 10 mins. The organic layer was separated and washed with brine. Then the layer was concentrated under reduce pressure to afford an oil. The oil, (3bS,4aR)-5,5-difluoro-3-(trifluoromethyl)-3b,4,4a,5-tetrahydro-1H-cyclopropa[3,4]cyclopenta[1,2-c]pyrazole(35 mg, 0.16 mmol) and K₂CO₃ (70 mg, 0.31 mmol) were dissolved into DMF (10 ml), and stirred for 2 hours at 40-45° C. After cooling, EtOAc (10 ml) and H₂O (15 ml) was added and stirred for 10 mins. The organic layer was separated and washed with brine twice. After concentration under reduce pressure and purification by preparative liquid phase chromatography (ACN: 0.5% TFA, SunFire^{@} Prep C18 OBD™, 5 μM×19 mm×150 mm), the title compound (22) was obtained, 9 mg. LCMS: [M+H]⁺:1004.1.

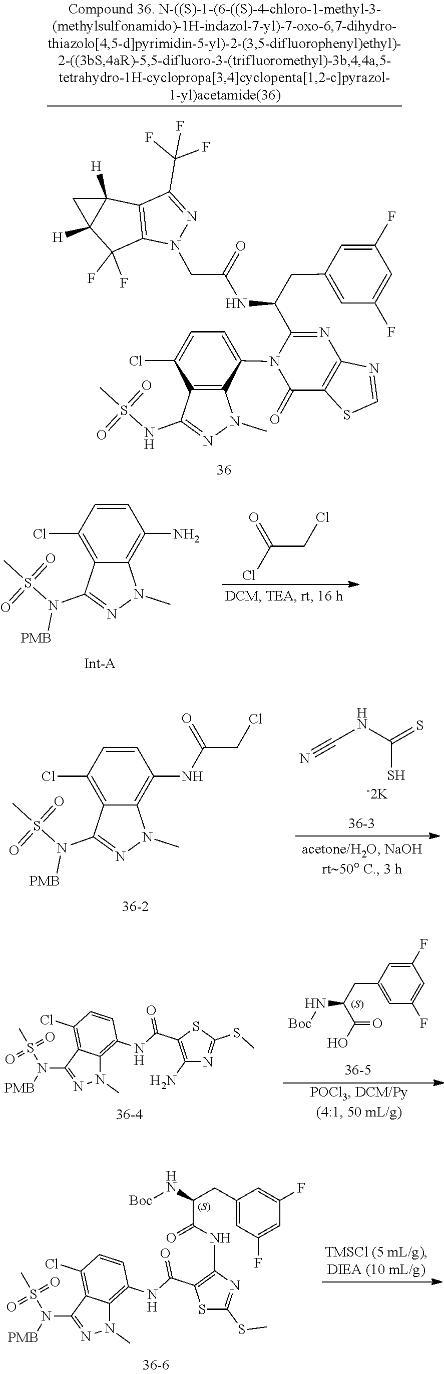

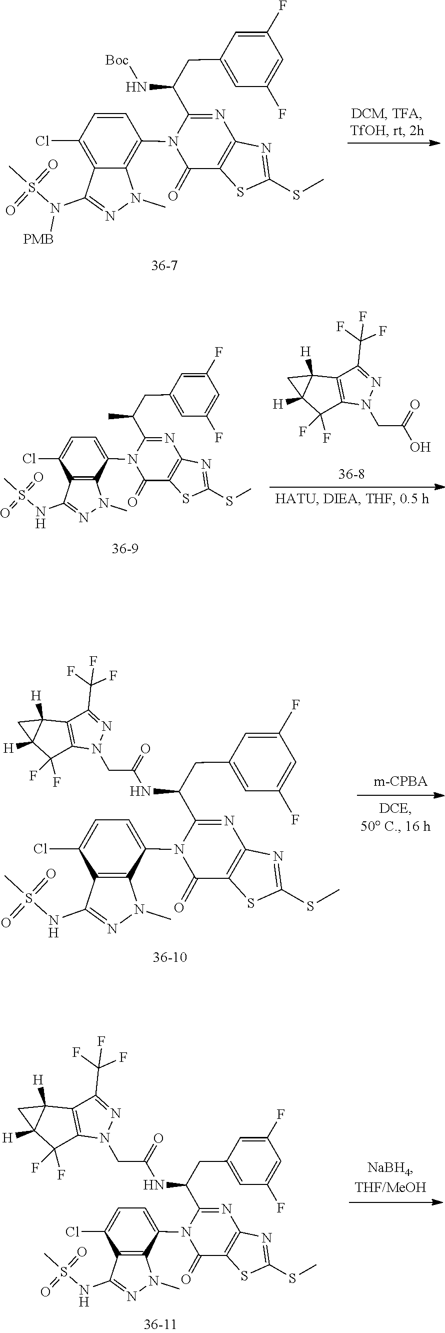

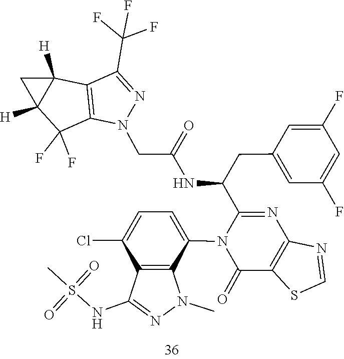

To a solution of N-(7-amino-4-chloro-1-methyl-1H-indazol-3-yl)-N-(4 methoxybenzyl)methanesulfonamide (10.0 g, 1.0 equiv) and TEA (3.9 g, 1.5 equiv) in DCM (100.0 mL) at 0° C., was added a solution of 2-chloroacetyl chloride (3.4 g, 1.2 equiv) in DCM (5.0 mL) slowly. Then, the reaction mixture was stirred at room temperature for 16 h. After completion of the reaction, the solvent is removed from the solution by evaporation. The crude product was purified by silica gel chromatography eluted with PE:EtOAc (0% to 10%) to give product (9.8 g) as a white solid.

To a solution of 2-chloro-N-(4-chloro-3-(N-(4-methoxybenzyl)methylsulfonamido)-1-methyl-1H-indazol-7-yl)acetamide (9.0 g, 1.0 equiv) and Dipotassium cyanodithioimidocarbonate (5 g, 0.8 equiv) in acetone (45.0 mL) and H₂O (45.0 mL) at 0° C., was added a solution of NaOH (0.4 g, 0.2 equiv) in H2O (4.0 mL). The mixture was stirred for 0.5 h at room temperature and then heated at 60° C. for 0.5 h. When the reaction was cooled to room temperature, MeI (2.7 g, 1.2 equiv) was added to it and stirring was continued for 1 h. After removal of acetone by evaporation, the mixture was extracted with EA (100.0 mL) three times. The separated organic layer was dried over Na₂SO₄ and evaporated. The solid was filtered and washed with petroleum ether to obtain the intermediate 4-amino-N-(4-chloro-3-(N-(4-methoxybenzyl)methylsulfonamido)-1-methyl-1H-indazol-7-yl)-2-(methylthio)thiazole-5-carboxamide (5.0 g) as a white solid.

To a solution of 4-amino-N-(4-chloro-3-(N-(4-methoxybenzyl)methylsulfonamido)-1-methyl-1H-indazol-7-yl)-2-(methylthio)thiazole-5-carboxamide (4.7 g, 1.0 equiv) and (S)-2-((tert-butoxycarbonyl)amino)-3-(3,5-difluorophenyl)propanoic acid (3.8 g, 1.5 equiv) in DCM (160.0 mL) at 0° C. under N₂ atmosphere, was added pyridine (40.0 mL). After stirring at 0° C. for 0.5 h, a solution of POCl3 (2.5 g, 2.0 equiv) in DCM (3.0 mL) was added slowly. Then, the reaction was stirred at room temperature for 2 h. After removal of solvent by evaporation. The crude product was purified by silica gel chromatography eluted with PE:EtOAc (0% to 50%) to give product (2.9 g) as a white solid.

To a solution of tert-butyl (S)-(1-((5-((4-chloro-3-(N-(4-methoxybenzyl)methylsulfonamido)-1-methyl-1H-indazol-7-yl)carbamoyl)-2-(methylthio)thiazol-4-yl)amino)-3-(3,5-difluorophenyl)-1-oxopropan-2-yl)carbamate (2.9 g, 1.0 equiv) in DCE (29.0 mL) at room temperature, was added DIEA (29.0 mL, 50.0 equiv) and TMSCl (14.5 mL, 50.0 equiv) slowly. Then, the reaction mixture was stirred at 90° C. for 1 h. After removal of solvent by evaporation, the crude product was purified by silica gel chromatography eluted with PE:EtOAc (0% to 45%) to give product (2.2 g) as a white solid.

To a solution of tert-butyl ((1S)-1-(6-(4-chloro-3-(N-(4-methoxybenzyl)methylsulfonamido)-1-methyl-1H-indazol-7-yl)-2-(methylthio)-7-oxo-6,7-dihydrothiazolo[4,5-d]pyrimidin-5-yl)-2-(3,5-difluorophenyl)ethyl)carbamate (2.2 g, 1.0 equiv) in DCM (15.0 mL) and TFA (5.0 mL) at room temperature, was added a solution of TfOH (2.0 g, 5.0 equiv) in DCM (2.0 mL). Then, the reaction mixture was stirred at room temperature for 1 h. After removal of solvent by evaporation, the crude was purified by SFC to give N—((S)-7-(5-((S)-1-amino-2-(3,5-difluorophenyl)ethyl)-2-(methylthio)-7-oxothiazolo[4,5-d]pyrimidin-6(7H)-yl)-4-chloro-1-methyl-1H-indazol-3-yl)methanesulfonamide (1.0 g) as a white solid.

To a solution of N—((S)-7-(5-((S)-1-amino-2-(3,5-difluorophenyl)ethyl)-2-(methylthio)-7-oxothiazolo[4,5-d]pyrimidin-6(7H)-yl)-4-chloro-1-methyl-1H-indazol-3-yl)methanesulfonamide (1.0 g, 1.0 equiv) and 2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetic acid (0.5 g, 1.2 equiv) in THF (10.0 mL) at room temperature, was added HATU (0.87 g, 1.5 equiv) and DIEA (0.79 g, 4.0 equiv). After stirring at room temperature for 1 h, the 2 N aqueous NaOH (10.0 mL) was added and the reaction was attired at room temperature for 15 mins. The mixture was extracted with EA (15.0 mL) three times. The separated organic layer was dried over Na₂SO₄ and evaporated to dryness. The crude product was purified by silica gel chromatography eluted with PE:EtOAc (0% to 60%) to give product (1.3 g) as a white solid.

To a solution of N-((1S)-1-(6-(4-chloro-1-methyl-3-(methylsulfonamido)-1H-indazol-7-yl)-2-(methylthio)-7-oxo-6,7-dihydrothiazolo[4,5-d]pyrimidin-5-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide (1.3 g, 1.0 equiv) in DCE (20.0 mL) at room temperature, was added m-CPBA (1.0 g, 4.0 equiv). Then, the reaction mixture was stirred at 50° C. for 16 h. After removal of solvent by evaporation, the crude product was purified by silica gel chromatography eluted with PE:EtOAc (0% to 50%) to give product (1.0 g) as a white solid.

To a solution of N—((S)-1-(6-((S)-4-chloro-1-methyl-3-(methylsulfonamido)-1H-indazol-7-yl)-2-(methylsulfonyl)-7-oxo-6,7-dihydrothiazolo[4,5-d]pyrimidin-5-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide (500 mg, 1.0 equiv) in THF (5.0 mL) and MeOH (5.0 mL) at 0° C., was added NaBH4 (1.8 g, 1.8 equiv). Then, the reaction mixture was stirred at room temperature for 0.5 h. After removal of solvent by evaporation, the material was purified by C18 reverse phase chromatography to give product 36 (259.0 mg) as a white solid.

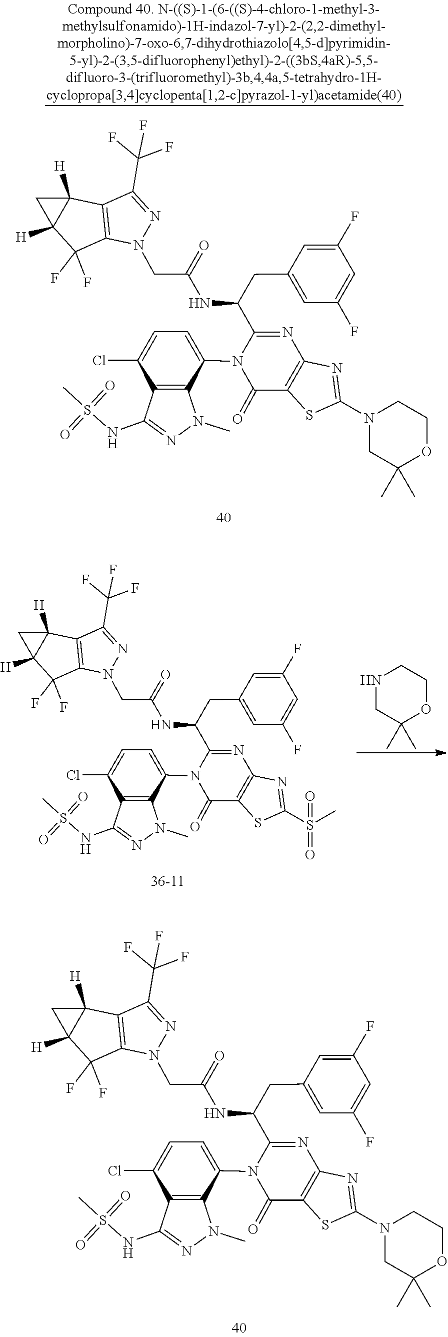

To a solution of N—((S)-1-(6-((S)-4-chloro-1-methyl-3-(methylsulfonamido)-1H-indazol-7-yl)-2-(methylsulfonyl)-7-oxo-6,7-dihydrothiazolo[4,5-d]pyrimidin-5-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide (500 mg, 1.0 equiv) in dioxane (5.0 mL) at room temperature, was added 2,2-dimethylmorpholine (317.3 mg, 5.0 equiv). Then, the reaction mixture was stirred at 80° C. for 1.0 h. After removal of solvent by evaporation, the material was purified by C18 reverse phase chromatography to give product 40 (410.0 mg) as a white solid.

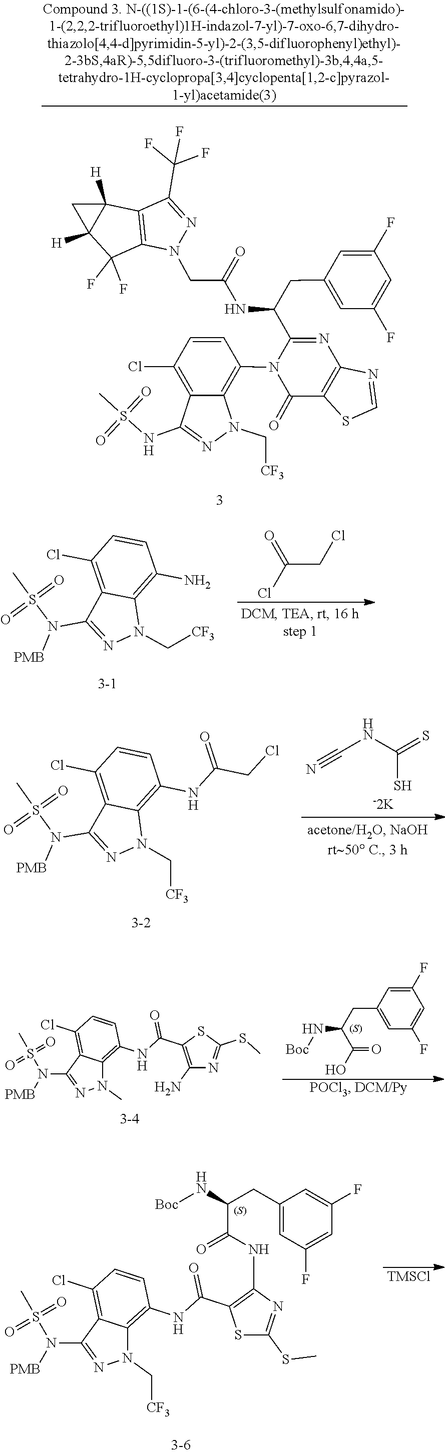

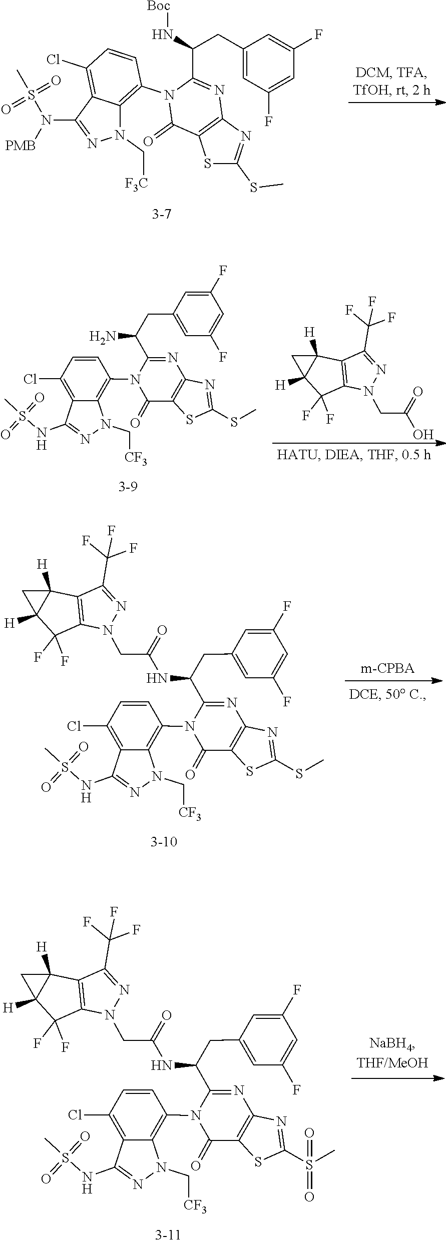

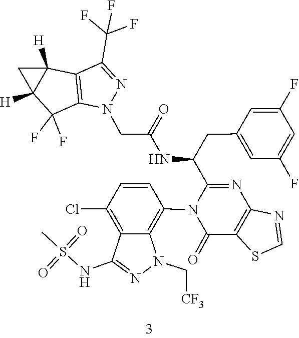 [SMILES: CS(=O)(=O)Nc1nn(CC(F)(F)F)c2c(-n3c([C@H](Cc4cc(F)cc(F)c4)NC(=O)Cn4nc(C(F)(F)F)c5c4C(F)(F)[C@@H]4C[C@H]54)nc4ncsc4c3=O)ccc(Cl)c12]

To a solution of N-(7-amino-4-chloro-1-(2,2,2-trifluoroethyl)-1H-indazol-3-yl)-N-(4-methoxybenzyl)methanesulfonamide (22.5 g, 1.0 equiv), TEA (12.3 g, 2.5 equiv) and DMAP (0.3 g, 0.05 equiv) in DCM (220.0 mL) at 0° C., was added a solution of 2-chloroacetyl chloride (11.0 g, 2.0 equiv) in DCM (8.0 mL) slowly. Then, the reaction mixture was stirred at room temperature for 16 h. After completion of the reaction, the solvent is removed from the solution by evaporation. The crude product was purified by silica gel chromatography eluted with PE:EtOAc (0% to 10%) to give product (19.5 g) as white solid.

To a solution of 2-chloro-N-(4-chloro-3-(N-(4-methoxybenzyl)methylsulfonamido)-1-(2,2,2-trifluoroethyl)-1H-indazol-7-yl)acetamide (19.5 g, 1.0 equiv) and Dipotassium cyanodithioimidocarbonate (10.8 g, 0.8 equiv) in acetone (100.0 mL) and H₂O (100.0 mL) at 0° C., was added a solution of NaOH (0.87 g, 0.2 equiv) in H₂O (8.0 mL). The mixture was stirred for 0.5 h at room temperature and then heated at 60° C. for 0.5 h. When the reaction was cooled to room temperature, MeI (8.8 g, 1.2 equiv) was added to it and stirring was continued for 1 h. After removal of acetone by evaporation, the mixture was extracted with EA (150.0 mL) three times. The separated organic layer was dried over Na₂SO₄ and evaporated. The solid was filtered and washed with petroleum ether to obtain the intermediate 4-amino-N-(4-chloro-3-(N-(4-methoxybenzyl)methylsulfonamido)-1-(2,2,2-trifluoroethyl)-1H-indazol-7-yl)-2-(methylthio)thiazole-5-carboxamide (13.2 g) as white solid.

To a solution of 4-amino-N-(4-chloro-3-(N-(4-methoxybenzyl)methylsulfonamido)-1-(2,2,2-trifluoroethyl)-1H-indazol-7-yl)-2-(methylthio)thiazole-5-carboxamide (12.1 g, 1.0 equiv) and (S)-2-((tert-butoxycarbonyl)amino)-3-(3,5-difluorophenyl)propanoic acid (8.6 g, 1.5 equiv) in DCM (240.0 mL) at 0° C. under N₂ atmosphere, was added pyridine (120.0 mL). After stirring at 0° C. for 0.5 h, a solution of POCl₃ (5.85 g, 2.0 equiv) in DCM (8.0 mL) was added slowly. Then, the reaction was stirred at room temperature for 2 h. After removal of solvent by evaporation, the crude product was purified by silica gel chromatography eluted with PE:EtOAc (0% to 50%) to give product (4.8 g) as a white solid.

To a solution of tert-butyl (S)-(1-((5-((4-chloro-3-(N-(4-methoxybenzyl)methylsulfonamido)-1-(2,2,2-trifluoroethyl)-1H-indazol-7-yl)carbamoyl)-2-(methylthio)thiazol-4-yl)amino)-3-(3,5-difluorophenyl)-1-oxopropan-2-yl)carbamate (4.8 g, 1.0 equiv) in DCE (48.0 mL) at room temperature, was added DIEA (48.0 mL, 50.0 equiv) and TMSCl (23.0 mL, 50.0 equiv) slowly. Then, the reaction mixture was stirred at 90° C. for 1 h. After removal of solvent by evaporation, the crude product was purified by silica gel chromatography eluted with PE:EtOAc (0% to 45%) to give product (2.9 g) as a white solid.

To a solution of tert-butyl ((1S)-1-(6-(4-chloro-3-(N-(4-methoxybenzyl)methylsulfonamido)-1-(2,2,2-trifluoroethyl)-1H-indazol-7-yl)-2-(methylthio)-7-oxo-6,7-dihydrothiazolo[4,5-d]pyrimidin-5-yl)-2-(3,5-difluorophenyl)ethyl)carbamate (2.9 g, 1.0 equiv) in DCM (29.0 mL) and TFA (10.0 mL) at room temperature, was added a solution of TfOH (2.4 g, 5.0 equiv) in DCM (3.0 mL). Then, the reaction mixture was stirred at room temperature for 1 h. After removal of solvent by evaporation, the crude was purified by C18 reverse phase chromatography to give product (1.5 g) as a white solid.

To a solution of N-(7-(5-((S)-1-amino-2-(3,5-difluorophenyl)ethyl)-2-(methylthio)-7-oxothiazolo[4,5-d]pyrimidin-6(7H)-yl)-4-chloro-1-(2,2,2-trifluoroethyl)-1H-indazol-3-yl)methanesulfonamide (766.0 mg, 1.0 equiv) and 2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetic acid (382.0 mg, 1.2 equiv) in THF (10.0 mL) at room temperature, was added HATU (601.0 mg, 1.5 equiv) and DIEA (583.0 g, 4.0 equiv). After stirring at room temperature for 1 h, the 2 N aqueous NaOH (10.0 mL) was added and the reaction was attired at room temperature for 15 min s. The mixture was extracted with EA (15.0 mL) three times. The separated organic layer was dried over Na₂SO₄ and evaporated to dryness. The crude product was purified by silica gel chromatography eluted with PE:EtOAc (0% to 65%) to give product (1.1 g) as a white solid.

To a solution of N-((1S)-1-(6-(4-chloro-3-(methylsulfonamido)-1-(2,2,2-trifluoroethyl)-1H-indazol-7-yl)-2-(methylthio)-7-oxo-6,7-dihydrothiazolo[4,5-d]pyrimidin-5-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide (1.1 g, 1.0 equiv) in DCE (20.0 mL) at room temperature, was added m-CPBA (0.95 g, 4.0 equiv). Then, the reaction mixture was stirred at 50° C. for 16 h. After removal of solvent by evaporation, the crude product was purified by silica gel chromatography eluted with PE:EtOAc (0% to 50%) to give product (1.0 g) as a white solid.

To a solution of N-((1S)-1-(6-(4-chloro-3-(methylsulfonamido)-1-(2,2,2-trifluoroethyl)-1H-indazol-7-yl)-2-(methylsulfonyl)-7-oxo-6,7-dihydrothiazolo[4,5-d]pyrimidin-5-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide (100 mg, 1.0 equiv) in THF(2.0 mL) and MeOH (2.0 mL) at 0° C., was added NaBH₄ (7.0 mg, 1.8 equiv). Then, the reaction mixture was stirred at room temperature for 0.5 h. After removal of solvent by evaporation, the material was purified by C18 reverse phase chromatography to give product (55.0 mg) as a white solid.

The following compounds (No. 1-53) were prepared according to the procedures described herein using the appropriate starting material(s) and intermediate(s) and appropriate protecting group chemistry as needed, and certified by ¹H NMR.

TABLE 2
The list of compounds

| | | MS | |
|---|---|---|---|
| No. | Structure | [M + H]⁺ | ¹H NMR |
| 1 | 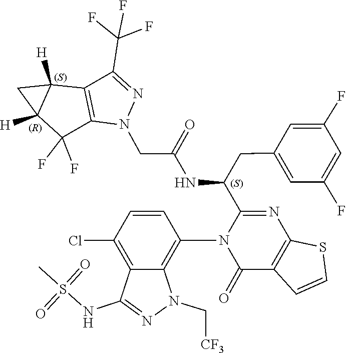 | 897.08 | ¹H NMR(400M, d-DMSO) δ: 9.98(s, 1H), 7.78-7.71(m, 2H), 7.44(d, J = 8.8 Hz, 1H), 7.39(d, J = 8.8 Hz, 1H), 7.03-6.96(m, 1H), 6.82(d, J = 8.0 Hz, 1H), 6.45- 6.42(m, 2H), 4.90-4.86(m, 1H), 4.68-4.64(m, 1H), 4.48-4.45(m, 2H), 3.92-3.90(m, 1H), 3.29- 3.19(m, 2H), 3.12(s, 3H), 2.97- 2.89(m, 2H), 1.37-1.35(m, 1H), 0.92-0.91(m, 1H). |
| 2 | 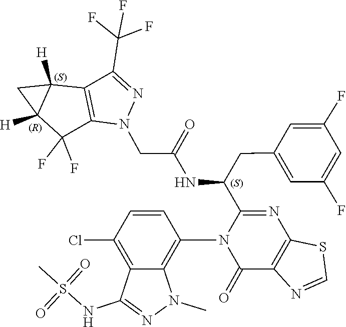 | 898.08 | ¹H NMR(400M, d-DMSO) δ: 9.97(s, 1H), 8.85(s, 1H), 7.78- 7.71(m, 2H), 7.03-6.96(m, 1H), 6.82(d, J = 8.0 Hz, 1H), 6.45- 6.42(m, 2H), 4.90-4.86(m, 1H), 4.68-4.64(m, 1H), 4.48-4.45(m, 2H), 3.92-3.90(m, 1H), 3.29- 3.19(m, 2H), 3.12(s, 3H), 2.97- 2.89(m, 2H), 1.37-1.35(m, 1H), 0.92-0.91(m, 1H). |
| 3 | 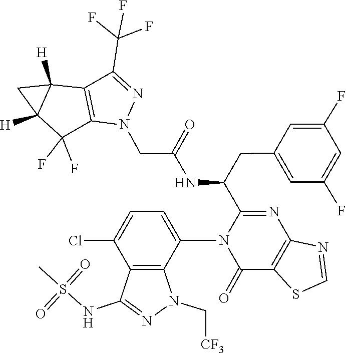 [SMILES: CS(=O)(=O)Nc1nn(CC(F)(F)F)c2c(-n3c([C@H](Cc4cc(F)cc(F)c4)NC(=O)Cn4nc(C(F)(F)F)c5c4C(F)(F)[C@@H]4C[C@H]54)nc4ncsc4c3=O)ccc(Cl)c12] | 898.08 | ¹H NMR(400M, d-DMSO) δ: 9.97(s, 1H), 9.07 (s, 1H), 7.78- 7.71(m, 2H), 7.03-6.96(m, 1H), 6.82(d, J = 8.0 Hz, 1H), 6.45- 6.42(m, 2H), 4.90-4.86(m, 1H), 4.68-4.64(m, 1H), 4.48-4.45(m, 2H), 3.92-3.90(m, 1H), 3.29- 3.19(m, 2H), 3.12(s, 3H), 2.97- 2.89(m, 2H), 1.37-1.35(m, 1H), 0.92-0.91(m, 1H). |
| 4 | 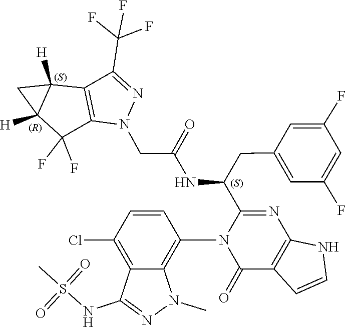 | 812.13 | ¹H NMR(400M, d-DMSO) δ: 11.2(d, J = 2.4 Hz, 1H), 9.98(s, 1H), 7.78-7.71(m, 2H), 7.21(d, J = 6.4 Hz, 1H), 7.03-6.96(m, 1H), 6.82(d, J = 8.0 Hz, 1H), 6.45- 6.42(m, 2H), 6.37(d, J = 6.4 Hz, 1H), 4.48-4.45(m, 2H), 4.05(s, 3H), 3.92-3.90(m, 1H), 3.29- 3.19(m, 2H), 3.12(s, 3H), 2.97- 2.89(m, 2H), 1.37-1.35(m, 1H), 0.92-0.91(m, 1H). |
| 5 | 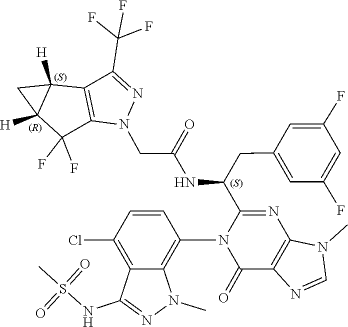 | 827.14 | ¹H NMR(400M, d-DMSO) δ: 9.98(s, 1H), 7.93(s, 1H), 7.78- 7.71(m, 2H), 7.03-6.96(m, 1H), 6.82(d, J = 8.0 Hz, 1H), 6.45- 6.42(m, 2H), 4.48-4.45(m, 2H), 4.05(s, 3H), 3.92-3.90(m, 1H), 3.76(s, 3H), 3.29-3.19(m, 2H), 3.12(s, 3H), 2.97-2.89(m, 2H) 1.37-1.35(m, 1H), 0.92-0.91(m, 1H) |
| 6 | 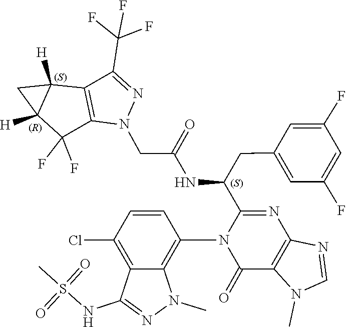 | 827.14 | ¹H NMR(400M, d-DMSO) δ: 9.96(s, 1H), 7.85 (s, 1H), 7.78- 7.71(m, 2H), 7.03-6.96(m, 1H), 6.82(d, J = 8.0 Hz, 1H), 6.45- 6.42(m, 2H), 4.48-4.45(m, 2H), 4.01(s, 3H), 3.92-3.90(m, 1H), 3.65(s, 3H), 3.29-3.19(m, 2H), 3.12(s, 3H), 2.97-2.89(m, 2H), 1.37-1.35(m, 1H), 0.92-0.91(m, 1H). |
| 7 | 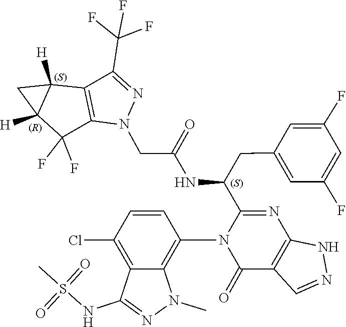 | 813.13 | ¹H NMR(400M, d-DMSO) δ: 12.65(s, 1H), 9.98(s, 1H), 8.33(s, 1H), 7.78-7.71(m, 2H), , 7.03- 6.96(m, 1H), 6.82(d, J = 8.0 Hz, 1H), 6.45-6.42(m, 2H), 4.48- 4.45(m, 2H), 4.05(s, 3H), 3.92- 3.90(m, 1H), 3.29-3.19(m, 2H), 3.12(s, 3H), 2.97-2.89(m, 2H), 1.37-1.35(m, 1H), 0.92-0.91(m, 1H). |
| 8 | 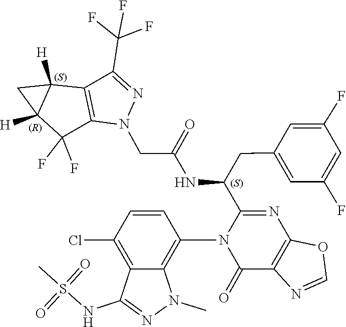 | 814.11 | ¹H NMR(400M, d-DMSO) δ: 9.98(s, 1H), 8.82(s, 1H), 7.78- 7.71(m, 2H), 7.03-6.96(m, 1H), 6.82(d, J = 8.0 Hz, 1H), 6.45- 6.42(m, 2H), 4.48-4.45(m, 2H), 4.05(s, 3H), 3.92-3.90(m, 1H), 3.29-3.19(m, 2H), 3.12(s, 3H), 2.97-2.89(m, 2H), 1.37-1.35(m, 1H), 0.92-0.91(m, 1H). |
| 9 | 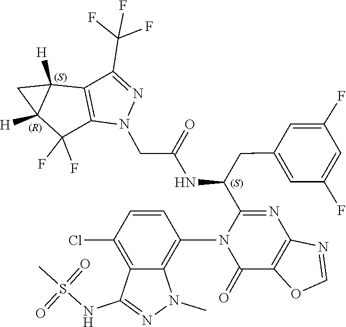 | 814.11 | ¹H NMR(400M, d-DMSO) δ: 9.98(s, 1H), 8.32(s, 1H), 7.78- 7.71(m, 2H), 7.03-6.96(m, 1H), 6.82(d, J = 8.0 Hz, 1H), 6.45- 6.42(m, 2H), 4.48-4.45(m, 2H), 4.05(s, 3H), 3.92-3.90(m, 1H), 3.29-3.19(m, 2H), 3.12(s, 3H), 2.97-2.89(m, 2H), 1.37- 1.35(m, 1H), 0.92-0.91(m, 1H). |
| 10 | 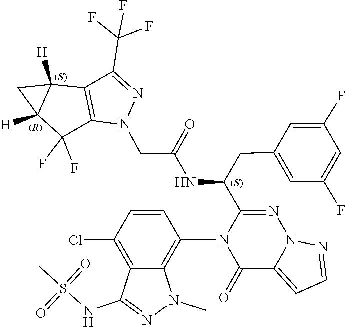 | 813.13 | ¹H NMR(400M, d-DMSO) δ: 9.98(s, 1H), 7.78-7.71(m, 2H), 7.44(d, J = 6.4 Hz, 1H), 7.19(d, J = 6.4 Hz, 1H), 7.03-6.96(m, 1H), 6.82(d, J = 8.0 Hz, 1H), 6.45- 6.42(m, 2H), 4.48-4.45(m, 2H), 4.05(s, 3H), 3.92-3.90(m, 1H), 3.29-3.19(m, 2H), 3.12(s, 3H), 2.97-2.89(m, 2H), 1.37-1.35(m, 1H), 0.92-0.91(m, 1H). |
| 11 | 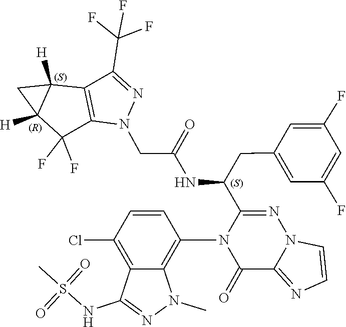 | 813.13 | ¹H NMR(400M, d-DMSO) δ: 9.98(s, 1H), 7.84(d, J = 8.0 Hz, 1H), 7.78-7.71(m, 2H), 7.69(d, J = 8.0 Hz, 1H), 7.03-6.96(m, 1H), 6.82(d, J = 8.0 Hz, 1H), 6.45- 6.42(m, 2H), 4.48-4.45(m, 2H), 4.05(s, 3H), 3.92-3.90(m, 1H), 3.29-3.19(m, 2H), 3.12(s, 3H), 2.97-2.89(m, 2H), 1.37-1.35(m, 1H), 0.92-0.91(m, 1H). |
| 12 | 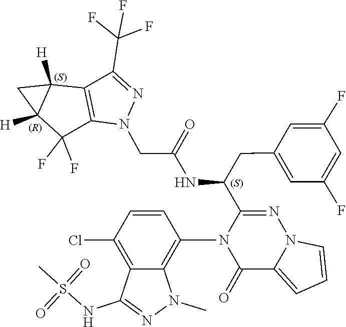 | 812.13 | ¹H NMR(400M, d-DMSO) δ: 9.98(s, 1H), 7.78-7.71(m, 2H), 7.59-7.58(m, 1H), 7.23-7.21(m, 1H), 7.03-6.96(m, 1H), 6.82(d, J = 8.0 Hz, 1H), 6.45-6.42(m, 2H), 6.20-6.19(m, 1H), 4.48-4.45(m, 2H), 4.05(s, 3H), 3.92-3.90(m, 1H), 3.29-3.19(m, 2H), 3.12(s, 3H), 2.97-2.89(m, 2H), 1.37- 1.35(m, 1H), 0.92-0.91(m, 1H). |
| 13 | 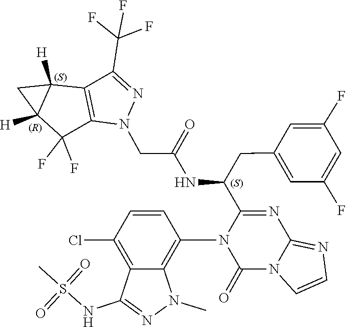 | 812.13 | ¹H NMR(400M, d-DMSO) δ: 9.98(s, 1H), 7.78-7.71(m, 2H), 7.63(d, J = 8.0 Hz, 1H), 7.42(d, J = 8.0 Hz, 1H), 7.03-6.96(m, 1H), 6.82(d, J = 8.0 Hz, 1H), 6.45- 6.42(m, 2H), 4.48-4.45(m, 2H), 4.05(s, 3H), 3.92-3.90(m, 1H), 3.29-3.19(m, 2H), 3.12(s, 3H), 2.97-2.89(m, 2H), 1.37- 1.35(m, 1H), 0.92-0.91(m, 1H). |
| 14 | 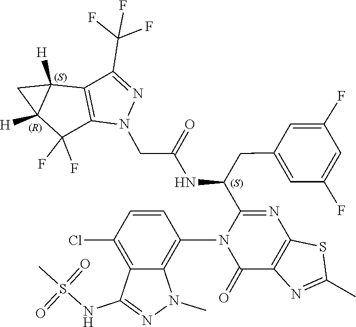 | 844.10 | ¹H NMR(400M, d-DMSO) δ: 9.98(s, 1H), 7.78-7.71(m, 2H), 7.03-6.96(m, 1H), 6.82(d, J = 8.0 Hz, 1H), 6.45-6.42(m, 2H), 4.48-4.45(m, 2H), 4.05(s, 3H), 3.92-3.90(m, 1H), 3.29-3.19(m, 2H), 3.12(s, 3H), 3.02(s, 3H), 2.97-2.89(m, 2H), 1.37-1.35(m, 1H), 0.92-0.91(m, 1H). |
| 15 | 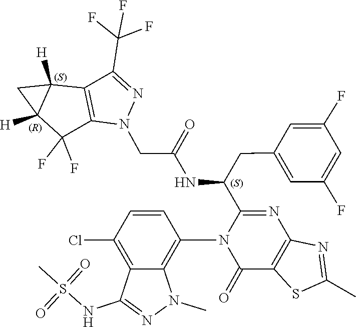 | 844.10 | ¹H NMR(400M, d-DMSO) δ: 9.98(s, 1H), 7.78-7.71(m, 2H), 7.03-6.96(m, 1H), 6.82(d, J = 8.0 Hz, 1H), 6.45-6.42(m, 2H), 4.48-4.45(m, 2H), 4.05(s, 3H), 3.92-3.90(m, 1H) , 3.29-3.19(m, 2H), 3.12(s, 3H), 2.98(s, 3H), 2.97-2.89(m, 2H), 1.37-1.35(m, 1H), 0.92-0.91(m, 1H). |
| 16 | 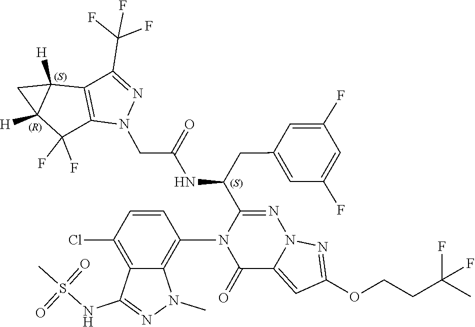 | 921.17 | ¹H NMR(400M, d-DMSO) δ: 9.98(s, 1H), 7.78-7.71(m, 2H), 7.17(d, J = 6.4 Hz, 1H), 7.03- 6.96(m, 1H), 6.82(d, J = 8.0 Hz, 1H), 6.45-6.42(m, 2H), 4.48- 4.45(m, 2H), 4.12(d, J = 6.4 Hz, 2H), 4.05(s, 3H), 3.92-3.90(m, 1H), 3.29-3.19(m, 2H), 3.12(s, 3H), 2.97-2.89(m, 2H), 2.22(d, J = 6.4 Hz, 2H), 1.83(s, 3H), 1.37- 1.35(m, 1H), 0.92-0.91(m, 1H). |
| 17 | 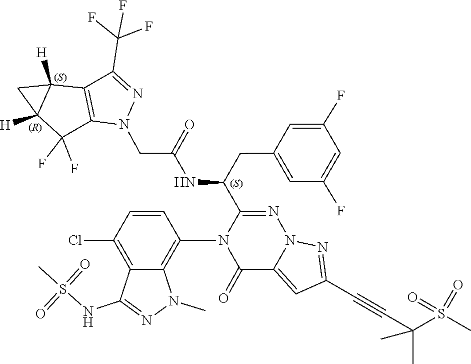 | 957.15 | ¹H NMR(400M, d-DMSO) δ: 9.98(s, 1H), 7.78-7.71(m, 2H), 7.25(d, J = 6.4 Hz, 1H), 7.03- 6.96(m, 1H), 6.82(d, J = 8.0 Hz, 1H), 6.45-6.42(m, 2H), 4.48- 4.45(m, 2H), 4.05(s, 3H), 3.92- 3.90(m, 1H), 3.29-3.19(m, 5H), 3.12(s, 3H), 2.97-2.89(m, 2H), 1.71(s, 6H), 1.37-1.35(m, 1H), 0.92-0.91(m, 1H). |
| 18 | 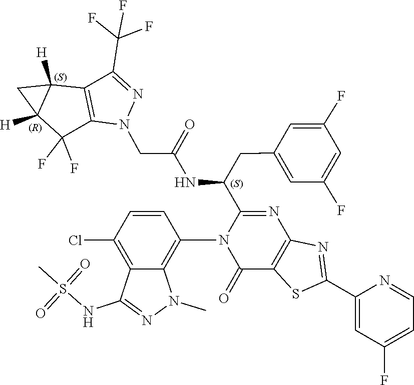 | 925.11 | ¹H NMR(400M, d-DMSO) δ: 9.98(s, 1H), 8.63-8.59(m, 1H), 7.78-7.71(m, 2H), 7.43-7.39(m, 1H), 7.03-6.92(m, 2H), 6.82(d, J = 8.0 Hz, 1H), 6.45-6.42(m, 2H), 4.48-4.45(m, 2H), 4.05(s, 3H), 3.92-3.90(m, 1H), 3.29-3.19(m, 2H), 3.12(s, 3H), 2.97-2.89(m, 2H), 1.37-1.35(m, 1H), 0.92- 0.91(m, 1H). |
| 19 | 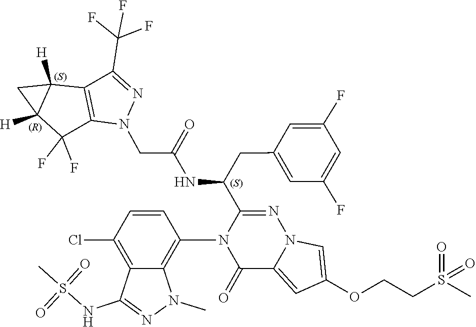 | 934.14 | ¹H NMR(400M, d-DMSO) δ: 9.98(s, 1H), 7.78-7.71(m, 2H), 7.61(s, 1H), 7.03-6.96(m, 1H), 6.82(d, J = 8.0 Hz, 1H), 6.74(s, 1H), 6.45-6.42(m, 2H), 4.48- 4.40(m, 4H), 4.05(s, 3H), 3.92- 3.90(m, 1H), 3.42(d, J = 7.6Hz, 2H), 3.29-3.19(m, 2H), 3.15(s, 3H), 3.12(s, 3H), 2.97-2.89(m, 2H), 1.37-1.35(m, 1H), 0.92- 0.91(m, 1H). |
| 20 | 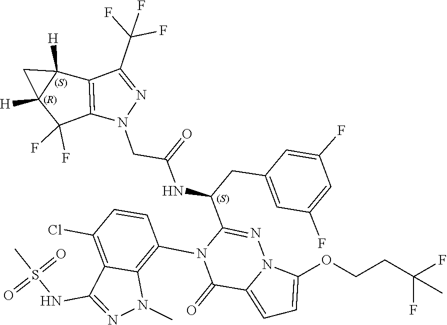 | 920.17 | ¹H NMR(400M, d-DMSO) δ: 9.98(s, 1H), 7.78-7.71(m, 2H), 7.61(s, 1H), 7.03-6.96(m, 1H), 6.82(d, J = 8.0 Hz, 1H), 6.74(s, 1H), 6.45-6.42(m, 2H), 4.48- 4.45(m, 2H), 4.23(d, J = 7.6Hz, 2H), 4.05(s, 3H), 3.92-3.90(m, 1H), 3.19 (d, J = 7.6Hz, 2H), 3.29-3.19(m, 2H), 3.15(s, 3H), 3.12(s, 3H), 2.97-2.89(m, 2H), 1.83(s, 3H), 1.37-1.35(m, 1H), 0.92-0.91(m, 1H). |
| 21 | 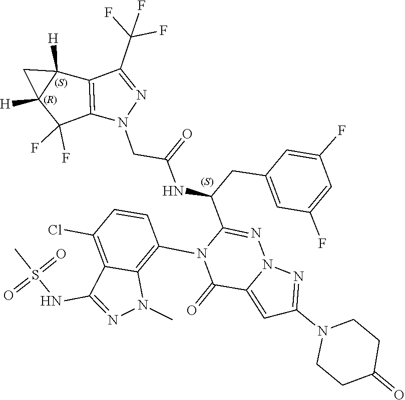 | 910.18 | ¹H NMR(400M, d-DMSO) δ: 9.98(s, 1H), 7.78-7.71(m, 2H), 7.40(d, J = 6.4 Hz, 1H), 7.03- 6.96(m, 1H), 6.82(d, J = 8.0 Hz, 1H), 6.45-6.42(m, 2H), 4.48- 4.45(m, 2H), 4.05(s, 3H), 3.92- 3.90(m, 1H), 3.59-3.55(m, 4H), 3.29-3.19(m, 2H), 3.12(s, 3H), 2.66-2.62(m, 4H), 2.97- 2.89(m, 2H), 1.37-1.35(m, 1H), 0.92-0.91(m, 1H). |
| 22 | 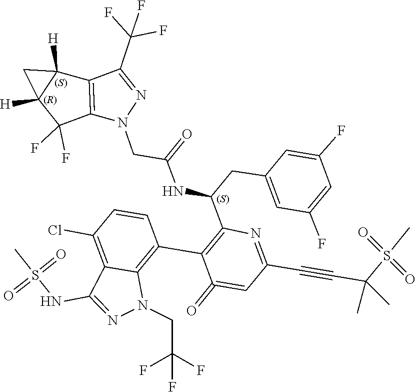 | 1004.11 | ¹H NMR(400M, d-DMSO) δ: 9.98(s, 1H), 9.21(d, J = 8.0 Hz, 1H), 7.78-7.71(m, 2H), 7.27(d, J = 8.0 Hz, 1H), 7.03-6.96(m, 1H), 6.82(d, J = 8.0 Hz, 1H), 6.45- 6.42(m, 2H), 5.11-5.07(m, 2H), 4.90-4.86(m, 1H), 4.68-4.64(m, 1H), 4.48-4.45(m, 2H), 3.92- 3.90(m, 1H), 3.21(s, 3H), 1.72(s, 6H), 3.12(s, 3H), 3.29-3.19(m, 2H), 1.37-1.35(m, 1H), 0.92- 0.91(m, 1H). |
| 23 | 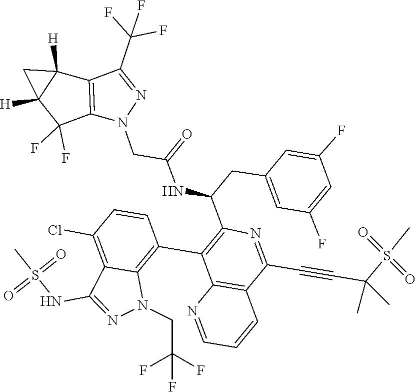 [SMILES: CC(C)(C#Cc1nc([C@H](Cc2cc(F)cc(F)c2)NC(=O)Cn2nc(C(F)(F)F)c3c2C(F)(F)[C@@H]2C[C@H]32)c(-c2ccc(Cl)c3c(NS(C)(=O)=O)nn(CC(F)(F)F)c23)c2ncccc12)S(C)(=O)=O] | 1019.15 | ¹H NMR(400M, d-DMSO) δ: 9.98(s, 1H), 8.68-8.65(m, 1H), 8.23-8.21(m, 1H), 7.78-7.71(m, 2H), 7.67-7.64(m, 1H), 7.03- 6.96(m, 1H), 6.82(d, J = 8.0 Hz, 1H), 6.45-6.42(m, 2H), 3.01- 2.95(m, 2H), 4.90-4.86(m, 1H), 4.68-4.64(m, 1H), 4.48-4.45(m, 2H), 3.92-3.90(m, 1H), 3.21(s, 3H), 1.72(s, 6H), 3.12(s, 3H), 3.29-3.19(m, 2H), 1.37-1.35(m, 1H), 0.92-0.91(m, 1H). |
| 24 | 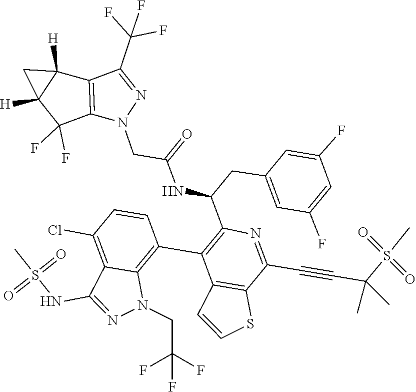 [SMILES: CC(C)(C#Cc1nc([C@H](Cc2cc(F)cc(F)c2)NC(=O)Cn2nc(C(F)(F)F)c3c2C(F)(F)[C@@H]2C[C@H]32)c(-c2ccc(Cl)c3c(NS(C)(=O)=O)nn(CC(F)(F)F)c23)c2ccsc12)S(C)(=O)=O] | 1024.12 | ¹H NMR(400M, d-DMSO) δ: 9.98(s, 1H), 8.05 (d, J = 4.8 Hz, 1H), 7.78-7.71(m, 3H), 7.03- 6.96(m, 1H), 6.82(d, J = 8.0 Hz, 1H), 6.45-6.42(m, 2H), 3.01- 2.95(m, 2H), 4.90-4.86(m, 1H), 4.68-4.64(m, 1H), 4.48-4.45(m, 2H), 3.92-3.90(m, 1H), 3.21(s, 3H), 1.72(s, 6H), 3.12(s, 3H), 3.29-3.19(m, 2H), 1.37-1.35(m, 1H), 0.92-0.91(m, 1H). |
| 25 | 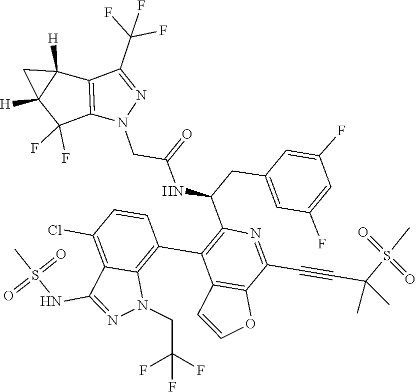 [SMILES: CC(C)(C#Cc1nc([C@H](Cc2cc(F)cc(F)c2)NC(=O)Cn2nc(C(F)(F)F)c3c2C(F)(F)[C@@H]2C[C@H]32)c(-c2ccc(Cl)c3c(NS(C)(=O)=O)nn(CC(F)(F)F)c23)c2ccoc12)S(C)(=O)=O] | 1008.14 | ¹H NMR(400M, d-DMSO) δ: 9.98(s, 1H), 7.97 (d, J = 4.8 Hz, 1H), 7.78-7.71(m, 2H), 7.67 (d, J = 4.8 Hz, 1H), 7.03-6.96(m, 1H), 6.82(d, J = 8.0 Hz, 1H), 6.45- 6.42(m, 2H), 3.01-2.95(m, 2H), 4.90-4.86(m, 1H), 4.68-4.64(m, 1H), 4.48-4.45(m, 2H), 3.92- 3.90(m, 1H), 3.21(s, 3H), 1.72(s, 6H), 3.12(s, 3H), 3.29-3.19(m, 2H), 1.37-1.35(m, 1H), 0.92- 0.91(m, 1H). |
| 26 | 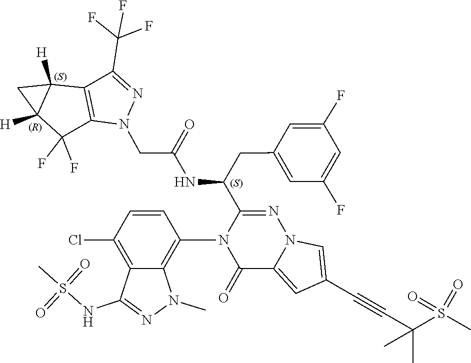 | 956.16 | ¹H NMR(400M, d-DMSO) δ: 9.98(s, 1H), 7.78-7.71(m, 2H), 7.27(s, 1H), 7.11(s, 1H), 7.03- 6.96(m, 1H), 6.82(d, J = 8.0 Hz, 1H), 6.45-6.42(m, 2H), 4.48- 4.45(m, 2H), 4.05(s, 3H), 3.92- 3.90(m, 1H), 3.29-3.19(m, 5H), 3.12(s, 3H), 2.97-2.89(m, 2H), 1.71(s, 6H), 1.37-1.35(m, 1H), 0.92-0.91(m, 1H). |
| 27 | 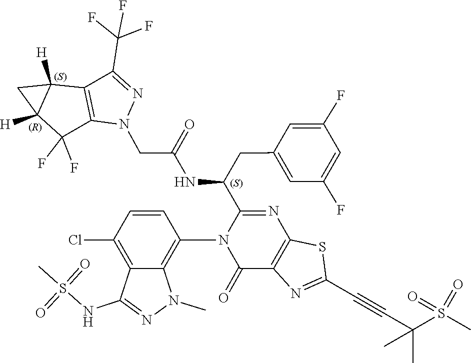 | 974.11 | ¹H NMR(400M, d-DMSO) δ: 9.98(s, 1H), 7.78-7.71(m, 2H), 7.03-6.96(m, 1H), 6.82(d, J = 8.0 Hz, 1H), 6.45-6.42(m, 2H), 4.48-4.45(m, 2H), 4.05(s, 3H), 3.92-3.90(m, 1H), 3.29-3.19(m, 5H), 3.12(s, 3H), 2.97-2.89(m, 2H), 1.71(s, 6H), 1.37-1.35(m, 1H), 0.92-0.91(m, 1H). |
| 28 | 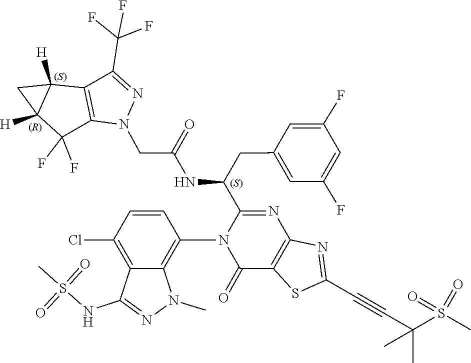 | 974.11 | ¹H NMR(400M, d-DMSO) δ: 9.97(s, 1H), 7.78-7.71(m, 2H), 7.03-6.97(m, 1H), 6.82(d, J = 8.0 Hz, 1H), 6.45-6.42(m, 2H), 4.48-4.45(m, 2H), 4.04(s, 3H), 3.92-3.90(m, 1H), 3.29-3.19(m, 5H), 3.12(s, 3H), 2.97-2.89(m, 2H), 1.71(s, 6H), 1.37-1.35(m, 1H), 0.92-0.91(m, 1H). |
| 29 | 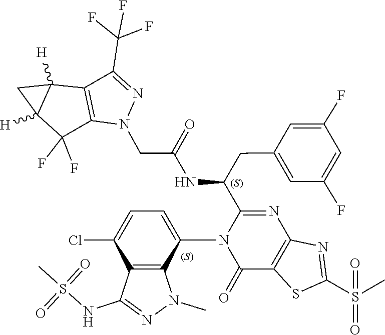 | 908.07 | ¹H NMR(400M, d-DMSO) δ: 9.86(s, 1H), 9.80(s, 1H), 7.46(d, J = 4.0 Hz, 1H), 7.04-6.99(m, 1H), 6.63-6.59(m, 2H), 4.76-4.72(m, 1H), 4.60-4.56(m, 1H), 4.51- 4.47(m, 1H), 3.49(s, 3H), 3.44(s, 3H), 3.38-3.35(m, 1H), 3.15(s, 3H), 3.03-2.97(m, 1H), 2.64- 2.53(m, 2H), 1.38-1.32(m, 1H), 0.88-0.87(m, 1H) |
| 30 | 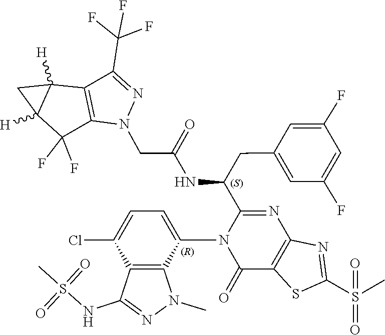 | 908.07 | ¹H NMR(400M, d-DMSO) δ: 9.86(s, 1H), 9.80(s, 1H), 7.46(d, J = 4.0 Hz, 1H), 7.04-6.99(m, 1H), 6.63-6.59(m, 2H), 4.76-4.72(m, 1H), 4.60-4.56(m, 1H), 4.51- 4.47(m, 1H), 3.49(s, 3H), 3.44(s, 3H), 3.38-3.35(m, 1H), 3.15(s, 3H), 3.03-2.97(m, 1H), 2.64- 2.53(m, 2H), 1.38-1.32(m, 1H), 0.88-0.87(m, 1H) |
| 31 | 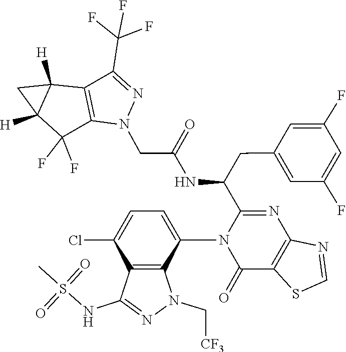 | 898.08 | ¹H NMR(400M, d-DMSO) δ: 9.81(s, 1H), 9.04(d, J = 4.0 Hz, 1H), 7.83(d, J = 8.0 Hz, 1H), 7.58(d, J = 8.0 Hz, 1H), 7.36(d, J = 8.0 Hz, 1H), 7.04-6.99(m, 1H), 6.67-6.64(m, 2H), 4.61- 4.42(m, 1H), 3.51(s, 3H), 3.37- 3.33(m, 1H), 3.14(s, 3H), 2.96- 2.90(m, 1H), 2.57-2.53(m, 2H), 1.40-1.35(m, 1H), 0.90(s, 1H). |
| 32 | 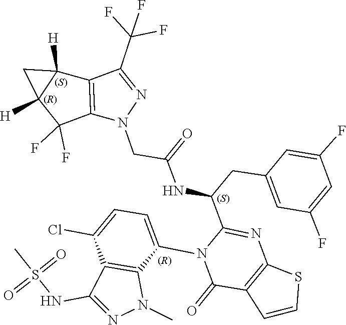 | 829.09 | ¹H NMR(400M, d-DMSO) δ: 9.86(s, 1H), 9.82(s, 1H), 7.63(d, J = 4.0 Hz, 1H), 7.46(d, J = 4.0 Hz, 1H), 7.37-7.35(m, 1H), 7.04- 6.99(m, 1H), 6.63-6.59(m, 2H), 4.76-4.72(m, 1H), 4.60-4.56(m, 1H), 4.51-4.47(m, 1H), 3.44(s, 3H), 3.38-3.35(m, 1H), 3.15(s, 3H), 3.03-2.97(m, 1H), 2.64- 2.53(m, 2H), 1.38-1.32(m, 1H), 0.88-0.87(m, 1H) |
| 33 | 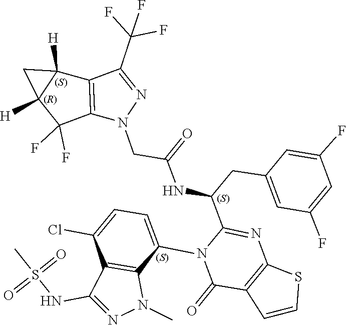 | 829.09 | ¹H NMR(400M, d-DMSO) δ: 9.86(s, 1H), 9.82(s, 1H), 7.63(d, J = 4.0 Hz, 1H), 7.46(d, J = 4.0 Hz, 1H), 7.37-7.35(m, 1H), 7.04- 6.99(m, 1H), 6.63-6.59(m, 2H), 4.76-4.72(m, 1H), 4.60-4.56(m, 1H), 4.51-4.47(m, 1H), 3.44(s, 3H), 3.38-3.35(m, 1H), 3.15(s, 3H), 3.03-2.97(m, 1H), 2.64- 2.53(m, 2H), 1.38-1.32(m, 1H), 0.88-0.87(m, 1H) |
| 34 | 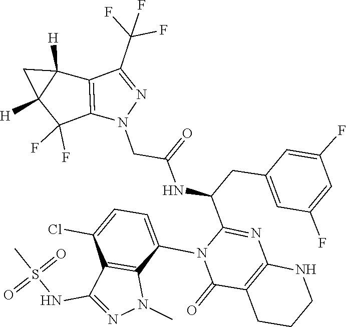 | 828.16 | ¹H NMR(400M, d-DMSO) δ: 9.86(s, 1H), 9.80(s, 1H), 9.21- 9.19(m, 1H), 7.46(d, J = 4.0 Hz, 1H), 7.04-6.99(m, 1H), 6.63- 6.59(m, 2H), 4.76-4.72(m, 1H), 4.60-4.56(m, 1H), 4.51-4.47(m, 1H), 3.44(s, 3H), 3.38-3.35(m, 3H), 3.15(s, 3H), 3.03-2.97(m, 1H), 2.64-2.53(m, 4H), 1.92- 1.89(m, 2H), 1.38-1.32(m, 1H), 0.88-0.87(m, 1H) |
| 35 | 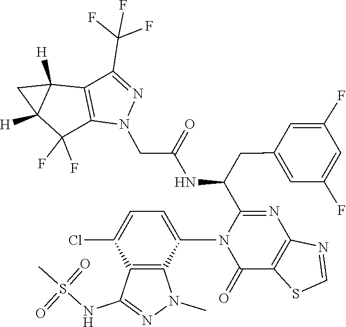 | 830.09 | ¹H NMR(400M, d-DMSO) δ: 9.86(s, 1H), 9.80(s, 1H), 7.73(d, J = 4.0 Hz, 1H), 7.46(d, J = 4.0 Hz, 1H), 7.04-6.99(m, 1H), 6.63- 6.59(m, 2H), 4.76-4.72(m, 1H), 4.60-4.56(m, 1H), 4.51-4.47(m, 1H), 3.44(s, 3H), 3.38-3.35(m, 1H), 3.15(s, 3H), 3.03-2.97(m, 1H), 2.64-2.53(m, 2H), 1.38- 1.32(m, 1H), 0.88-0.87(m, 1H) |
| 36 | 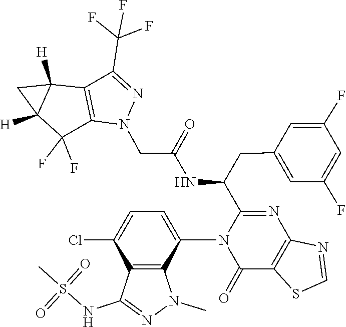 | 830.09 | ¹H NMR(400M, d-DMSO) δ: 9.86(s, 1H), 9.80(s, 1H), 7.73(d, J = 4.0 Hz, 1H), 7.46(d, J = 4.0 Hz, 1H), 7.04-6.99(m, 1H), 6.63- 6.59(m, 2H), 4.76-4.72(m, 1H), 4.60-4.56(m, 1H), 4.51-4.47(m, 1H), 3.44(s, 3H), 3.38-3.35(m, 1H), 3.15(s, 3H), 3.03-2.97(m, 1H), 2.64-2.53(m, 2H), 1.38- 1.32(m, 1H), 0.88-0.87(m, 1H) |
| 37 | 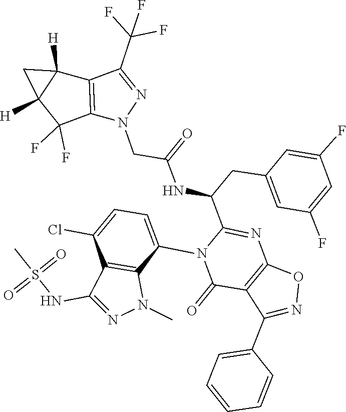 | 890.14 | ¹H NMR(400M, d-DMSO) δ: 9.89(s, 1H), 9.48(d, J = 8.0 Hz, 1H), 8.22-8.21(m, 2H), 7.75- 7.72(m, 1H), 7.61-7.50(m, 4H), 7.10-7.03(m, 1H), 6.69-6.66(m, 2H), 4.83-4.68(m, 2H), 4.55- 4.51(m, 1H), 3.56(s, 3H), 3.18(s, 3H), 3.07-3.01(m, 1H), 2.66- 2.56(m, 2H), 1.42-1.36(m, 1H), 0.93-0.86(m, 1H). |
| 38 | 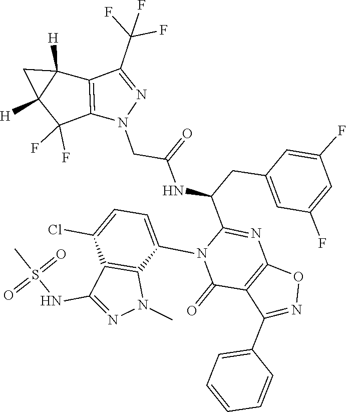 | 890.14 | ¹H NMR(400M, d-DMSO) δ: 9.89(s, 1H), 9.48(d, J = 8.0 Hz, 1H), 8.22-8.21(m, 2H), 7.75- 7.72(m, 1H), 7.61-7.50(m, 4H), 7.10-7.03(m, 1H), 6.69-6.66(m, 2H), 4.83-4.68(m, 2H), 4.55- 4.51(m, 1H), 3.56(s, 3H), 3.18(s, 3H), 3.07-3.01(m, 1H), 2.66- 2.56(m, 2H), 1.42-1.36(m, 1H), 0.93-0.86(m, 1H). |
| 39 | 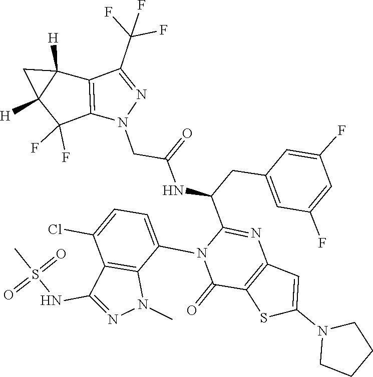 [SMILES: Cn1nc(NS(C)(=O)=O)c2c(Cl)ccc(-n3c([C@H](Cc4cc(F)cc(F)c4)NC(=O)Cn4nc(C(F)(F)F)c5c4C(F)(F)[C@@H]4C[C@H]54)nc4cc(N5CCCC5)sc4c3=O)c21] | 898.15 | ¹H NMR(400M, d-DMSO) δ: 9.86(s, 1H), 9.82(s, 1H), 7.46(d, J = 4.0 Hz, 1H), 7.37-7.35(m, 1H), 7.04-6.99(m, 1H), 6.63-6.59(m, 2H), 6.42(s, 1H), 4.76-4.72(m, 1H), 4.60-4.56(m, 1H), 3.65- 3.62(m, 4H), 4.51-4.47(m, 1H), 3.44(s, 3H), 3.38-3.35(m, 1H), 3.15(s, 3H), 3.03-2.97(m, 1H), 2.64-2.53(m, 2H), 2.05-1.99(m, 4H), 1.38-1.32(m, 1H), 0.88- 0.87(m, 1H). |
| 40 | 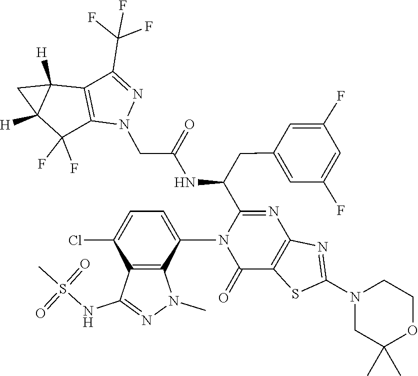 | 943.17 | ¹H NMR(400M, d-DMSO) δ: 9.82(s, 1H), 9.28(d, J = 8.0 Hz, 1H), 7.61(d, J = 8.0 Hz, 1H), 7.38(d, J = 8.0 Hz, 1H), 7.04- 6.99(m, 1H), 6.61-6.58(m, 2H), 4.73-4.69(m, 1H), 4.63-4.53(m, 1H), 4.44-4.41(m, 1H), 3.77- 3.75(m, 2H), 3.63-3.51(m, 3H), 3.33(s, 3H), 3.14(s, 3H), 2.96- 2.90(m, 1H), 2.62-2.48(m, 2H), 1.39-1.35(m, 1H), 1.20(s, 2H), 0.89(s, 1H). |
| 41 | 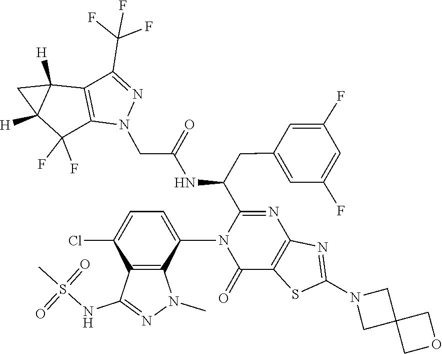 | 927.14 | ¹H NMR(400M, d-DMSO) δ: 9.81(s, 1H), 9.22(d, J = 8.0 Hz, 1H), 7.60(d, J = 8.0 Hz, 1H), 7.37(d, J = 8.0 Hz, 1H), 7.04- 6.99(m, 1H), 6.61-6.59(m, 2H), 4.73-4.67(m, 5H), 4.52- 4.47(m, 1H), 4.45-4.36(m, 5H), 3.41(s, 3H), 3.36-3.33(m, 1H), 3.13(s, 3H), 2.95-2.89(m, 1H), 2.63-2.51(m, 2H), 1.39-1.34(m, 1H), 0.89(s, 1H). |
| 42 | 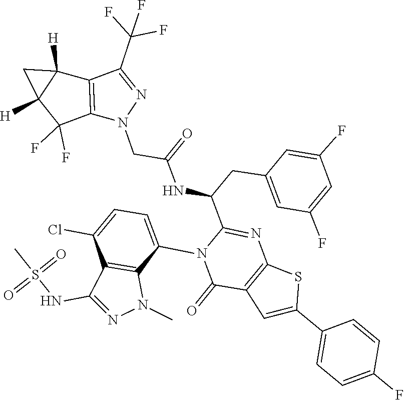 | 923.12 | ¹H NMR(400M, d-DMSO) δ: 9.86(s, 1H), 9.82(s, 1H), 7.87- 7.85(m, 2H), 7.76(d, J = 4.0 Hz, 1H), 7.37-7.35(m, 1H), 7.17- 7.15(m, 2H), 7.04-6.99(m, 1H), 6.63-6.59(m, 2H), 4.76-4.72(m, 1H), 4.60-4.56(m, 1H), 4.51- 4.47(m, 1H), 3.44(s, 3H), 3.38- 3.35(m, 1H), 3.15(s, 3H), 3.03- 2.97(m, 1H), 2.64-2.53(m, 2H), 1.38-1.32(m, 1H), 0.88-0.87(m, 1H) |
| 43 | 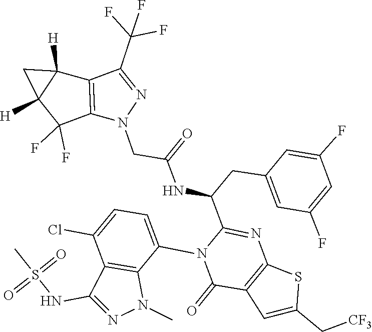 [SMILES: Cn1nc(NS(C)(=O)=O)c2c(Cl)ccc(-n3c([C@H](Cc4cc(F)cc(F)c4)NC(=O)Cn4nc(C(F)(F)F)c5c4C(F)(F)[C@@H]4C[C@H]54)nc4sc(CC(F)(F)F)cc4c3=O)c21] | 911.10 | ¹H NMR(400M, d-DMSO) δ: 9.86(s, 1H), 9.82(s, 1H), 7.46(d, J = 4.0 Hz, 1H), 7.37-7.35(m, 1H), 7.04-6.99(m, 1H), 6.63-6.59(m, 2H), 4.76-4.72(m, 1H), 4.60- 4.56(m, 1H), 4.51-4.47(m, 1H), 3.67-3.60(m, 2H), 3.44(s, 3H), 3.38-3.35(m, 1H), 3.15(s, 3H), 3.03-2.97(m, 1H), 2.64-2.53(m, 2H), 1.38-1.32(m, 1H), 0.88- 0.87(m, 1H) |
| 44 | 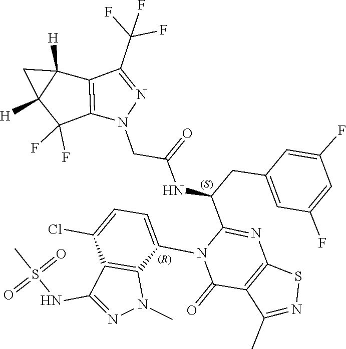 | 844.10 | ¹H NMR(400M, d-DMSO) δ: 9.86(s, 1H), 9.80(s, 1H), 7.46(d, J = 4.0 Hz, 1H), 7.04-6.99(m, 1H), 6.63-6.59(m, 2H), 4.76-4.72(m, 1H), 4.60-4.56(m, 1H), 4.51- 4.47(m, 1H), 3.44(s, 3H), 3.38- 3.35(m, 1H), 3.15(s, 3H), 3.03- 2.97(m, 1H), 2.71(s, 3H), 2.64- 2.53(m, 2H), 1.38-1.32(m, 1H), 0.88-0.87(m, 1H) |
| 45 | 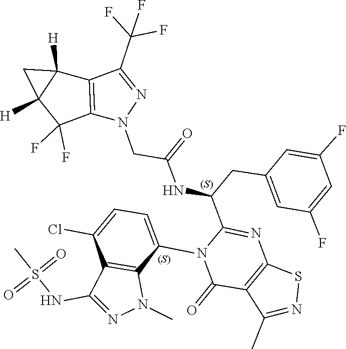 | 844.10 | ¹H NMR(400M, d-DMSO) δ: 9.86(s, 1H), 9.80(s, 1H), 7.46(d, J = 4.0 Hz, 1H), 7.04-6.99(m, 1H), 6.63-6.59(m, 2H), 4.76-4.72(m, 1H), 4.60-4.56(m, 1H), 4.51- 4.47(m, 1H), 3.44(s, 3H), 3.38- 3.35(m, 1H), 3.15(s, 3H), 3.03- 2.97(m, 1H), 2.71(s, 3H), 2.64- 2.53(m, 2H), 1.38-1.32(m, 1H), 0.88-0.87(m, 1H) |
| 46 | 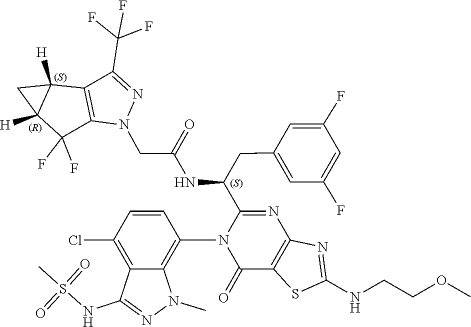 | 903.14 | ¹H NMR(400M, d-DMSO) δ: 9.81(s, 1H), 9.28(d, J = 8.0 Hz, 1H), 9.08(t, J = 4.0 Hz, 1H), 7.61(d, J = 8.0 Hz, 1H), 7.37(d, J = 8.0 Hz, 1H), 7.04-6.99(m, 1H), 6.61-6.58(m, 2H), 4.74- 4.70(m, 1H), 4.57-4.53(m, 1H), 4.43(t, J = 4.0 Hz, 1H), 3.57- 3.44(m, 4H), 3.35(s, 3H), 3.34- 3.28(m, 1H), 3.13(s, 3H), 2.99- 2.90(m, 1H), 2.54-2.48(m, 2H), 1.39-1.35(m, 1H), 0.91-0.87(m, 1H). |
| 47 | 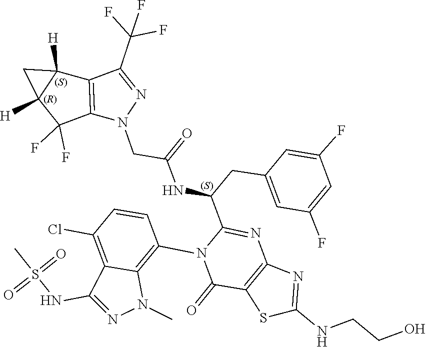 | 889.13 | ¹H NMR(400M, d-DMSO) δ: 9.81(s, 1H), 9.26(d, J = 8.0 Hz, 1H), 9.05(t, J = 4.0 Hz, 1H), 7.60(d, J = 8.0 Hz, 1H), 7.36(d, J = 8.0 Hz, 1H), 7.04-6.99(m, 1H), 6.60-6.58(m, 2H), 4.89- 4.86(m, 1H), 4.74-4.70(m, 1H), 4.57-4.53(m, 1H), 4.46-4.41(m, 1H), 3.61-3.58(m, 2H), 3.51- 3.48(m, 5H), 3.34-3.31(m, 1H), 3.13(s, 3H), 2.96-2.90(m, 1H), 2.63-2.48(m, 2H), 1.38-1.33(m, 1H), 0.91-0.87(m, 1H). |
| 48 | 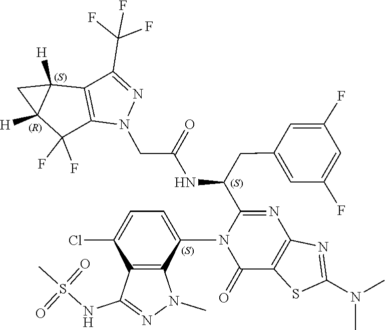 | 873.13 | ¹H NMR(400M, d-DMSO) δ: 9.81(s, 1H), 9.32(d, J = 8.0 Hz, 1H), 7.64(d, J = 8.0 Hz, 1H), 7.41(d, J = 8.0 Hz, 1H), 7.08- 7.02(m, 1H), 6.65-6.62(m, 2H), 4.77-4.73(m, 1H), 4.61-4.57(m, 1H), 4.48(t, J = 12.0 Hz, 1H), 3.46(s, 3H), 3.40-3.33(m, 1H), 3.24(s, 6H), 3.13(s, 3H), 3.00- 2.94(m, 1H), 2.67-2.53(m, 2H), 1.42-1.37(m, 1H), 0.94-0.92(m, 1H). |
| 49 | 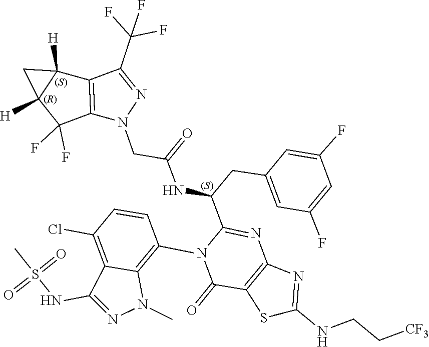 | 941.09 | ¹H NMR(400M, d-DMSO) δ: 9.84(s, 1H), 9.33(d, J = 8.0 Hz, 1H), 9.21(d, J = 8.0 Hz, 1H), 7.77(d, J = 8.0 Hz, 1H), 7.41(d, J = 8.0 Hz, 1H), 7.07-7.01(m, 1H), 6.63-6.61(m, 2H), 4.79- 4.74(m , 1H), 4.61-4.57(m, 1H), 4.46-4.43(m, 1H), 3.71-3.70(m, 2H), 3.47(s, 3H), 3.46-3.45(m, 1H), 3.17(s, 3H), 3.01-2.94(m, 1H), 2.74-2.68(m, 2H), 2.55- 2.50(m, 2H), 1.42-1.37(m, 1H), 0.94-0.92(m, 1H). |
| 50 | 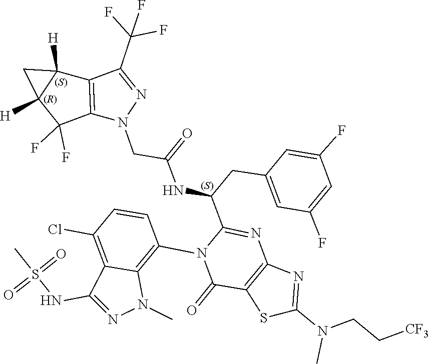 | 955.13 | ¹H NMR(400M, d-DMSO) δ: 9.84(s, 1H), 9.33(d, J = 8.0 Hz, 1H), 7.77(d, J = 8.0 Hz, 1H), 7.41(d, J = 8.0 Hz, 1H), 7.07- 7.01(m, 1H), 6.63-6.61(m, 2H), 4.79-4.74(m , 1H), 4.61-4.57(m, 1H), 4.46-4.43(m, 1H), 3.71- 3.70(m, 2H), 3.47(s, 3H), 3.34(s, 3H), 3.46-3.45(m, 1H), 3.17(s, 3H), 3.01-2.94(m, 1H), 2.74- 2.68(m, 2H), 2.55-2.50(m, 2H), 1.42-1.37(m, 1H), 0.94-0.92(m, 1H). |
| 51 | 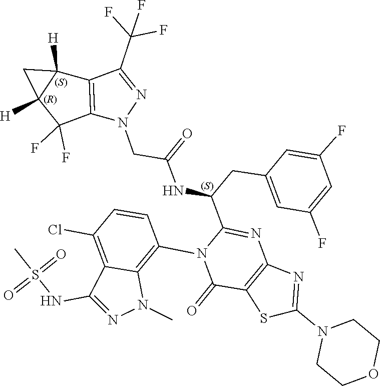 | 915.14 | ¹H NMR(400M, d-DMSO) δ: 9.83(s, 1H), 9.30(d, J = 8.0 Hz, 1H), 7.62(d, J = 8.0 Hz, 1H), 7.40(d, J = 8.0 Hz, 1H), 7.05- 76.99(m, 1H), 6.62-6.59(m, 2H), 4.74-4.70(m, 1H), 4.57- 4.53(m, 1H), 4.44-4.42(m, 1H), 3.75-3.72(m, 4H), 3.66-3.64(m, 4H), 3.43(s, 3H), 3.36-3.35(m, 1H), 3.14(s, 3H), 2.97-2.90(m, 1H), 2.63-2.46(m, 2H), 1.39- 1.33(m, 1H), 0.91-0.87(m, 1H). |
| 52 | 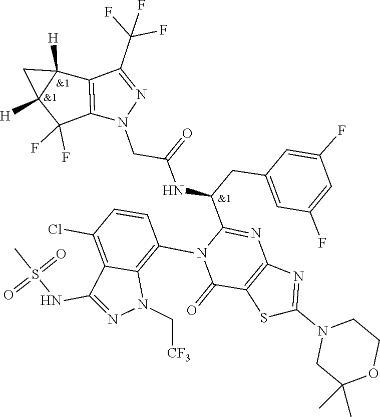 [SMILES: CC1(C)CN(c2nc3nc([C@H](Cc4cc(F)cc(F)c4)NC(=O)Cn4nc(C(F)(F)F)c5c4C(F)(F)[C@@H]4C[C@H]54)n(-c4ccc(Cl)c5c(NS(C)(=O)=O)nn(CC(F)(F)F)c45)c(=O)c3s2)CCO1] | 1011.16 | ¹H NMR(400M, d-DMSO) δ: 10.07(s, 1H), 9.31(d, J = 8.0 Hz, 1H), 7.73(d, J = 8.0 Hz, 1H), 7.55(d, J = 8.0 Hz, 1H), 7.07- 7.01(m, 1H), 6.61-6.58(m, 2H), 4.83-4.79(m, 1H), 4.65-4.61(m, 1H), 4.58-4.52(m, 1H), 4.44- 4.40(m, 1H), 4.23-4.17(m, 1H), 3.81-3.70(m, 2H), 3.70- 3.64(m, 3H), 3.44-3.40(m, 1H), 3.18(s, 3H), 2.96-2.90(m, 1H), 2.59-2.53(m, 2H), 1.42-1.37(m, 1H), 0.96-0.92(m, 1H). |
| 53 | 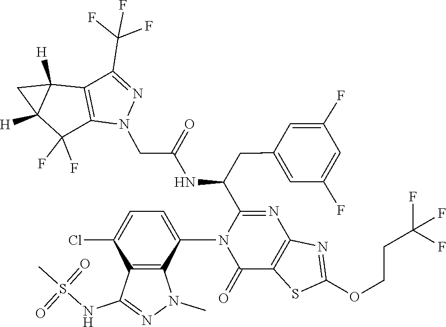 | 942.10 | ¹H NMR(400M, d-DMSO) δ: 9.81(s, 1H), 9.04(d, J = 4.0 Hz, 1H), 7.58(d, J = 8.0 Hz, 1H), 7.36(d, J = 8.0 Hz, 1H), 7.04- 6.99(m, 1H), 6.67-6.64(m, 2H), 4.61-4.42(m, 3H), 3.51(s, 3H), 3.37-3.33(m, 2H), 3.14(s, 3H), 2.96-2.90(m, 1H), 2.75-2.69(m, 1H), 2.57-2.53(m, 2H), 1.40- 1.35(m, 1H), 0.90(s, 1H). |

### Example 3 Biological Assays

The compounds were evaluated for inhibition of HIV-1IIIB infection and cellular toxicity in MT-4 cells.

### Cells and Virus

The MT-4 cells and HIV-1111 used in the HIV-1 inhibition assays were obtained from the AIDS Research and Reference Program (Rockville, MD) and propagated as recommended by the supplier.

### Evaluation of Inhibition of HIV-1_{IIIB} and Cellular Toxicity in MT-4 Cells

Fifty microliters (50 μL) of MT-4 cells at a density of 1.0×10⁴ cells/well in 10% complete RPMI-1640 (10% FBS supplemented with 1% L-glutamine and 1% Penicillin/Streptomycin) media were plated in a 96-well flat bottom plate. One-hundred microliters (100 μL) of each compound at 12 concentrations were added in triplicate followed by 50 μL of HIV-1_{IIIB} at a pre-determined titer. The cultures were incubated for 6 days at 37° C./5% CO₂. Following the incubation, the cells were stained with XTT for evaluation of compound efficacy and cellular toxicity, as described below.

### XTT Staining for Cell Viability and Compound Cytotoxicity:

Efficacy and toxicity values for the test materials were derived by measuring the reduction of the tetrazolium dye XTT (2,3-bis(2-methoxy-4-nitro-5-sulfophenyl)-5-[(phenylamino)carbonyl]-2H-tetrazolium hydroxide). XTT in metabolically active cells is metabolized by the mitochondrial enzyme NADPH oxidase to a soluble formazan product. XTT solution was prepared daily as a stock of 1 mg/mL in RPMI-1640 without additives. Phenazine methosulfate (PMS) solution was prepared at 0.15 mg/mL in DPBS and stored in the dark at −20° C. XTT/PMS stock was prepared immediately before use by adding 40 μL of PMS per mL of XTT solution. Fifty μL (50 μL) of XTT/PMS was added to each well of the plate and the plate incubated for 4 hours at 37° C. The 4-hour incubation has been empirically determined to be within the linear response range for XTT dye reduction with the indicated numbers of cells for each assay. The plates were sealed and inverted several times to mix the soluble formazan product and the plate was read at 450 nm (650 nm reference wavelength) with a Molecular Devices SpectraMax Plus 384 96 well plate format spectrophotometer.

### Data Analysis and Evaluation:

Microsoft Excel 2010 in combination with XLfit4 was used to analyze and graph the data. EC₅₀ (50% inhibition of virus replication), TC₅₀ (50% reduction in cell viability) and a therapeutic index (TC₅₀/EC₅₀) are provided. Raw data for both antiviral activity and toxicity with a graphic representation of the data are provided in a printout summarizing the compound activity.

### Result:

Table 3: Efficacy and Toxicity of Seven Compounds against HIV-1_{IIIB} in MT-4 Cells

| | EC₅₀ | TC₅₀ | Therapeutic |
|---|---|---|---|
| COMPOUND | (nM) | (nM) | Index |
| 1 | 0.32 | >100 | >312.5 |
| 3 | 0.16 | >100 | >625.0 |
| 29 | 34.9 | >100 | >2.9 |
| 30 | >100 | >100 | N/A |
| 31 | 0.26 | >100 | >384.6 |
| 32 | >100 | >100 | N/A |
| 33 | 0.34 | >100 | >294.1 |
| 34 | 2.35 | >100 | >42.6 |
| 35 | >100 | >100 | N/A |
| 36 | 0.16 | >100 | >625.0 |
| 37 | 11.1 | >100 | >9.0 |
| 38 | >100 | >100 | N/A |
| 39 | 18.6 | >100 | >5.4 |
| 40 | 0.203 | >100 | >492.6 |
| 41 | 0.517 | >100 | >193.4 |
| 42 | 3.79 | >100 | >26.4 |
| 43 | 3.15 | >100 | >31.7 |
| 44 | 34.1 | >100 | >2.9 |
| 45 | 0.47 | >100 | >212.8 |
| 46 | 0.196 | >100 | >510.2 |
| 47 | 1.20 | >100 | >83.3 |
| 48 | 0.231 | >100 | >432.9 |
| 49 | 0.218 | >100 | >458.7 |
| 50 | 0.088 | >100 | >1136.4 |
| 51 | 0.193 | >100 | >518.1 |
| 52 | 0.153 | >100 | >653.6 |
| 53 | 0.102 | >100 | >980.4 |

What is claimed is:

1. A compound of Formula I: 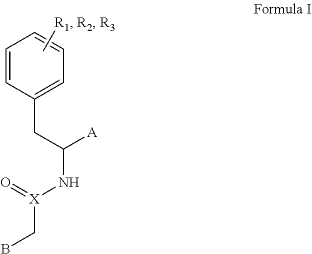
or stereoisomers thereof, or pharmaceutically acceptable salts thereof, or deuterium substitutes thereof,
wherein each of R₁, R₂, and R₃ is independently selected from the group consisting of H, Cl, F, -OMe, —CN, C₁-C₃ alkyl, and C₃-C₅ cycloalkyl, wherein the C₁-C₃ alkyl is optionally substituted with 1-3 halo;
wherein X is C, S, S═O, or P—R₁₀;
wherein R₁₀ is selected from the group consisting of H, OH, C₁-C₃ alkyl, C₃-C₅ cycloalkyl, —O—C₁-C₃ alkyl, —O—C₃-C₅ cycloalkyl, —NH—C₁-C₃ alkyl, and —NH—C₃-C₅ cycloalkyl;
wherein A is 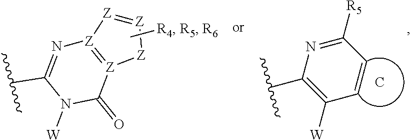
wherein each Z is independently selected from the group consisting of C, CH, N, NH, O, and S;
wherein each of R₄, R₅ and R₆ is independently selected from the group consisting of H, halogen, C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, C₃₋₁₅ cycloalkyl, heterocyclyl, C₆₋₁₀ aryl, 5-10 membered heteroaryl, oxo, —NO₂, —N₃, —CN, —NH(C₁₋₉ alkyl), —NH(C₁₋₈ haloalkyl), —NH(C₂₋₆ alkenyl), —NH(C₂₋₆ alkynyl), —NH(C₃₋₁₅ cycloalkyl), —NH(heterocyclyl), —NH(C₆₋₁₀ aryl), —NH(5-10 membered heteroaryl), —N(cycloalkyl)₂, —N(C₁₋₉ alkyl)₂, —N(C₁₋₈ haloalkyl)₂, —N(C₂₋₆ alkenyl)₂, —N(C₂₋₆ alkynyl)₂, —N(C₃₋₁₅ cycloalkyl)₂, —N(heterocyclyl)₂, —N(C₆₋₁₀ aryl)₂, —N(5-10 membered heteroaryl)₂, —N(C₁₋₉ alkyl)(C₁₋₈ haloalkyl), —N(C₁₋₉ alkyl)(C₂₋₆ alkenyl), —N(C₁₋₉ alkyl)(C₂₋₆ alkynyl), —N(C₁₋₉ alkyl)(C₃₋₁₅ cycloalkyl), —N(C₁₋₉ alkyl)(heterocyclyl), —N(C₁₋₉ alkyl)(C₆₋₁₀ aryl), —N(C₁₋₉ alkyl)(5-10 membered heteroaryl), —SH, —O(C₁₋₉ alkyl), —O(C₁₋₈ haloalkyl), —O(C₂₋₆ alkenyl), —O(C₂₋₆ alkynyl), —O(C₃₋₁₅ cycloalkyl), —O(heterocyclyl), —O(C₆₋₁₀ aryl), and −0(5-10 membered heteroaryl);
wherein each of R₄, R₅ and R₆ is optionally substituted with one or more substituents each independently selected from the group consisting of halo, C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, C₃₋₁₅ cycloalkyl, heterocyclyl, C₆₋₁₀ aryl, 5-10 membered heteroaryl, oxo, —NO₂, —N₃, —CN, —S(O)— C₁₋₉ alkyl, —S(O)₂— C₁₋₉ alkyl, —S(O)—C₃₋₁₅ cycloalkyl, and —S(O)₂— C₃₋₁₅ cycloalkyl, wherein the C₁₋₉ alkyl and C₃₋₁₅ cycloalkyl are optionally substituted with one or more halo;
wherein ring C is 5-10 membered heteroaryl; wherein the 5-10 membered heteroaryl is optionally substituted with one or more substituents each independently selected from the group consisting of halo, C₁₋₉ alkyl, C₁₋₈ haloalkyl, C₁₋₆ alkoxy, and C₁₋₆ haloalkoxy;
wherein W is 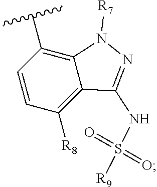
wherein R₇ is C₁-C₃ alkyl, or C₃₋₅ cycloalkyl; wherein R₇ is optionally substituted with one to nine halo;
wherein R₈ is selected from the group consisting of H, Cl, F, -OMe, —CN, —C₁-C₃ alkyl, and —C₃-C₅ cycloalkyl, wherein the C₁-C₃ alkyl is optionally substituted with 1-3 fluorines;
wherein R₉ is C₁-C₃ alkyl, or C₃-C₅ cycloalkyl; wherein R₉ is optionally substituted with 1-3 fluorines; and
wherein B is 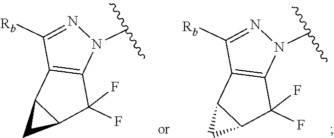
wherein R_{b} is C₁-C₃ alkyl, or C₃₋₅ cycloalkyl; wherein R_{b} is optionally substituted with one to nine halo.

2. The compound of claim 1, wherein A is selected from the group consisting of

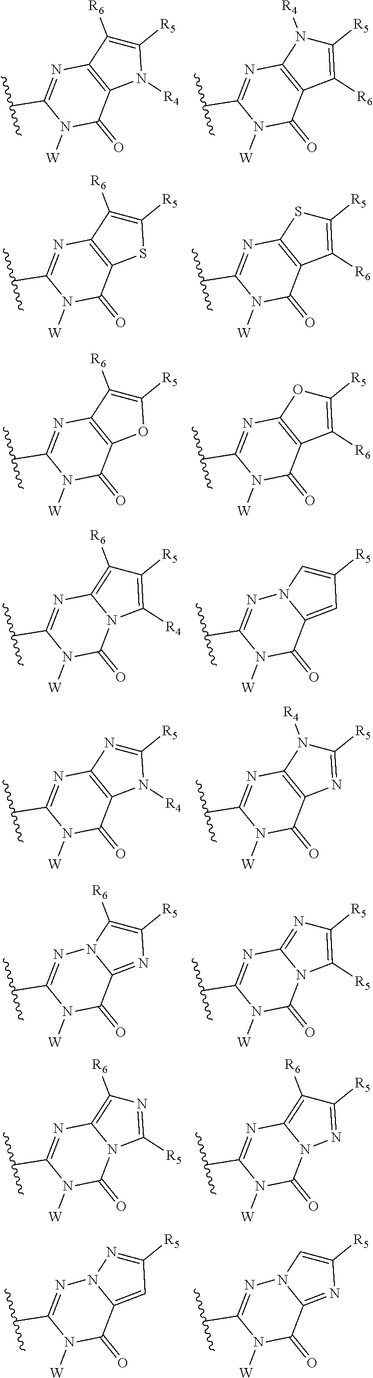

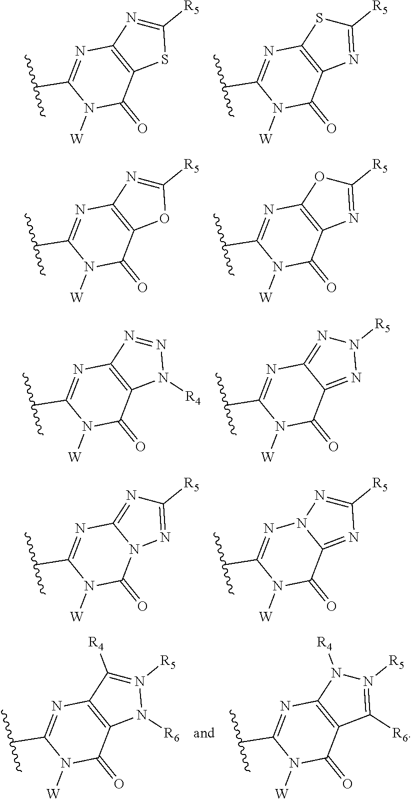

3. The compound of claim 1, wherein A is

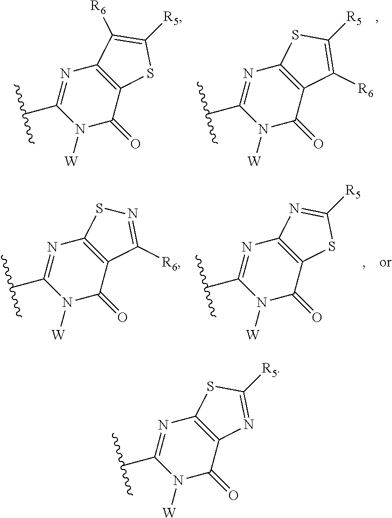

4. The compound of claim 1, wherein A is

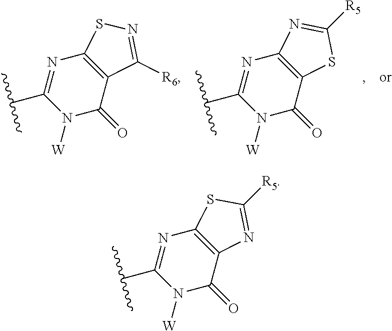

5. The compound of claim 1, wherein each of R₁, R₂, and R₃ is independently selected from the group consisting of H, Cl, F.

6. The compound of claim 1, wherein X is C.

7. The compound of claim 1, wherein B is

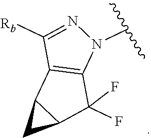

8. The compound of claim 1, wherein B is

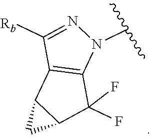

9. The compound of claim 1, wherein W is 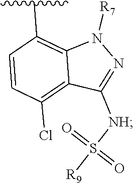
wherein R₇ is C₁-C₃ alkyl, or C₃₋₅ cycloalkyl; wherein R₇ is optionally substituted with one to nine halo;
wherein R₉ is C₁-C₃ alkyl, or C₃-C₅ cycloalkyl; wherein R₉ is optionally substituted with 1-3 fluorines.

10. The compound of claim 1, wherein the compound has the structure of Formula II, 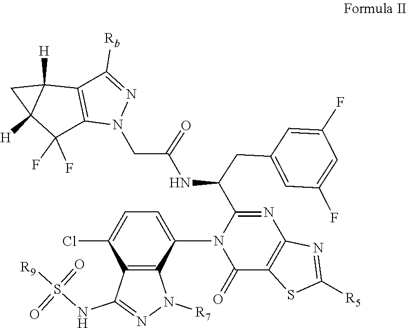
wherein R₇ is C₁-C₃ alkyl, or C₃₋₅ cycloalkyl; wherein R₇ is optionally substituted with one to nine halo;
wherein R₉ is C₁-C₃ alkyl, or C₃-C₅ cycloalkyl; wherein R₉ is optionally substituted with 1-3 fluorines;
wherein R_{b} is C₁-C₃ alkyl, or C₃₋₅ cycloalkyl; wherein R_{b} is optionally substituted with one to nine halo;
wherein R₅ is independently selected from the group consisting of H, halogen, C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, C₃₋₁₅ cycloalkyl, heterocyclyl, C₆₋₁₀ aryl, 5-10 membered heteroaryl, oxo, —NO₂, —N₃, —CN, —NH(C₁₋₉ alkyl), —NH(C₁₋₈ haloalkyl), —NH(C₂₋₆ alkenyl), —NH(C₂₋₆ alkynyl), —NH(C₃₋₁₅ cycloalkyl), —NH(heterocyclyl), —NH(C₆₋₁₀ aryl), —NH(5-10 membered heteroaryl), —N(cycloalkyl)₂, —N(C₁₋₉ alkyl)₂, —N(C₁₋₈ haloalkyl)₂, —N(C₂₋₆ alkenyl)₂, —N(C₂₋₆ alkynyl)₂, —N(C₃₋₁₅ cycloalkyl)₂, —N(heterocyclyl)₂, —N(C₆₋₁₀ aryl)₂, —N(5-10 membered heteroaryl)₂, —N(C₁₋₉ alkyl)(C₁₋₈ haloalkyl), —N(C₁₋₉ alkyl)(C₂₋₆ alkenyl), —N(C₁₋₉ alkyl)(C₂₋₆ alkynyl), —N(C₁₋₉ alkyl)(C₃₋₁₅ cycloalkyl), —N(C₁₋₉ alkyl)(heterocyclyl), —N(C₁₋₉ alkyl)(C₆₋₁₀ aryl), —N(C₁₋₉ alkyl)(5-10 membered heteroaryl), —SH, —O(C₁₋₉ alkyl), —O(C₁₋₈ haloalkyl), —O(C₂₋₆ alkenyl), —O(C₂₋₆ alkynyl), —O(C₃₋₁₅ cycloalkyl), —O(heterocyclyl), —O(C₆₋₁₀ aryl), and —O(5-10 membered heteroaryl);
wherein R₅ is optionally substituted with one or more substituents each independently selected from the group consisting of halo, C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, C₃₋₁₅ cycloalkyl, heterocyclyl, C₆₋₁₀ aryl, 5-10 membered heteroaryl, oxo, —NO₂, —N₃, —CN, —S(O)— C₁₋₉ alkyl, —S(O)₂— C₁₋₉ alkyl, —S(O)— C₃₋₁₅ cycloalkyl, and —S(O)₂— C₃₋₁₅ cycloalkyl, wherein the C₁₋₉ alkyl and C₃₋₁₅ cycloalkyl are optionally substituted with one or more halo.

11. The compound of claim 10, wherein R₅ is independently selected from the group consisting of H, halogen, C₁₋₉ alkyl, C₁₋₈ haloalkyl, C₁₋₆ alkoxy, C₁₋₆ haloalkoxy, C₂₋₆ alkoxyalkyl, C₃₋₁₅ cycloalkyl, heterocyclyl, C₆₋₁₀ aryl, 5-10 membered heteroaryl, oxo, —NO₂, —N₃, —CN, —NH(C₁₋₉ alkyl), —NH(C₁₋₈ haloalkyl), —NH(C₂₋₆ alkenyl), —NH(C₂₋₆ alkynyl), —NH(C₃₋₁₅ cycloalkyl), —NH(heterocyclyl), —NH(C₆₋₁₀ aryl), —NH(5-10 membered heteroaryl), —N(cycloalkyl)₂, —N(C₁₋₉ alkyl)₂, —N(C₁₋₈ haloalkyl)₂, —N(C₂₋₆ alkenyl)₂, —N(C₂₋₆ alkynyl)₂, —N(C₃₋₁₅ cycloalkyl)₂, —N(heterocyclyl)₂, —N(C₆₋₁₀ aryl)₂, —N(5-10 membered heteroaryl)₂, —N(C₁₋₉ alkyl)(C₁₋₈ haloalkyl), —N(C₁₋₉ alkyl)(C₂₋₆ alkenyl), —N(C₁₋₉ alkyl)(C₂₋₆ alkynyl), —N(C₁₋₉ alkyl)(C₃₋₁₅ cycloalkyl), —N(C₁₋₉ alkyl)(heterocyclyl), —N(C₁₋₉ alkyl)(C₆₋₁₀ aryl), —N(C₁₋₉ alkyl)(5-10 membered heteroaryl), —O(C₁₋₉ alkyl), —O(C₁₋₈ haloalkyl), —O(C₂₋₆ alkenyl), —O(C₂₋₆ alkynyl), —O(C₃₋₁₅ cycloalkyl), —O(heterocyclyl);
wherein R₅ is optionally substituted with one or more substituents each independently selected from the group consisting of halo, C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, C₃₋₁₅ cycloalkyl, heterocyclyl, C₆₋₁₀ aryl, 5-10 membered heteroaryl, oxo, —NO₂, —N₃, —CN, —S(O)— C₁₋₉ alkyl, —S(O)₂— C₁₋₉ alkyl, —S(O)—C₃₋₁₅ cycloalkyl, and —S(O)₂— C₃₋₁₅ cycloalkyl, wherein the C₁₋₉ alkyl and C₃₋₁₅ cycloalkyl are optionally substituted with one or more halo.

12. The compound of claim 10, wherein R₅ is independently selected from the group consisting of H, halogen, C₁₋₉ alkyl, C₁₋₈ haloalkyl, C₁₋₆ alkoxy, C₁₋₆ haloalkoxy, C₂₋₆ alkoxyalkyl, C₃₋₁₅ cycloalkyl, heterocyclyl, C₆₋₁₀ aryl, 5-10 membered heteroaryl, oxo, —NO₂, —N₃, —CN, —NH(C₁₋₉ alkyl), —NH(C₁₋₈ haloalkyl), —NH(C₂₋₆ alkenyl), —NH(C₂₋₆ alkynyl), —NH(C₃₋₁₅ cycloalkyl), —NH(heterocyclyl), —NH(C₆₋₁₀ aryl), —NH(5-10 membered heteroaryl), —N(cycloalkyl)₂, —N(C₁₋₉ alkyl)₂, —N(C₁₋₉ haloalkyl)₂, —N(C₂₋₆ alkenyl)₂, —N(C₂₋₆ alkynyl)₂, —N(C₃₋₁₅ cycloalkyl)₂, —N(heterocyclyl)₂, —N(C₆₋₁₀ aryl)₂, —N(5-10 membered heteroaryl)₂, —N(C₁₋₉ alkyl)(C₁₋₈ haloalkyl), —N(C₁₋₉ alkyl)(C₂₋₆ alkenyl), —N(C₁₋₉ alkyl)(C₂₋₆ alkynyl), —N(C₁₋₉ alkyl)(C₃₋₁₅ cycloalkyl), —N(C₁₋₉ alkyl)(heterocyclyl), —N(C₁₋₉ alkyl)(C₆₋₁₀ aryl), —N(C₁₋₉ alkyl)(5-10 membered heteroaryl), —O(C₁₋₉ alkyl), —O(C₁₋₈ haloalkyl), —O(C₂₋₆ alkenyl), —O(C₂₋₆ alkynyl), —O(C₃₋₁₅ cycloalkyl), —O(heterocyclyl);
wherein R₅ is optionally substituted with one or more substituents each independently selected from the group consisting of halo, C₁₋₉ alkyl, C₁₋₈ haloalkyl, C₁₋₆ alkoxy, C₁₋₆ haloalkoxy, C₃₋₁₅ cycloalkyl, heterocyclyl, C₆₋₁₀ aryl, 5-10 membered heteroaryl, wherein the C₁₋₉ alkyl and C₃₋₁₅ cycloalkyl are optionally substituted with one or more halo.

13. The compound of claim 10, wherein R₅ is independently selected from the group consisting of H, halogen, C₁₋₉ alkyl, C₁₋₈ haloalkyl, C₁₋₆ alkoxy, C₁₋₆ haloalkoxy, C₂₋₆ alkoxyalkyl, C₃₋₁₅ cycloalkyl, heterocyclyl, C₆₋₁₀ aryl, 5-10 membered heteroaryl, —NH(C₁₋₉ alkyl), —NH(C₁₋₈ haloalkyl), —NH(C₃₋₁₅ cycloalkyl), —NH(heterocyclyl), —N(cycloalkyl)₂, —N(C₁₋₉ alkyl)₂, —N(C₁₋₈ haloalkyl)₂, —N(C₃₋₁₅ cycloalkyl)₂, —N(C₁₋₉ alkyl)(C₁₋₈ haloalkyl), —N(C₁₋₉ alkyl)(C₃₋₁₅ cycloalkyl), —N(C₁₋₉ alkyl)(heterocyclyl), —N(C₁₋₉ alkyl)(C₆₋₁₀ aryl), —N(C₁₋₉ alkyl)(5-10 membered heteroaryl), —O(C₁₋₉ alkyl), —O(C₁₋₈ haloalkyl), —O(C₃₋₁₅ cycloalkyl), —O(heterocyclyl);
wherein R₅ is optionally substituted with one or more substituents each independently selected from the group consisting of halo, C₁₋₉ alkyl, C₁₋₈ haloalkyl, C₁₋₆ alkoxy, C₁₋₆ haloalkoxy, C₃₋₁₅ cycloalkyl, heterocyclyl, C₆₋₁₀ aryl, 5-10 membered heteroaryl, wherein the C₁₋₉ alkyl and C₃₋₁₅ cycloalkyl are optionally substituted with one or more halo.

14. The compound of claim 4, or pharmaceutically acceptable salts thereof, or deuterium substitutes thereof, or isomers thereof, or prodrugs thereof, or metabolites thereof, wherein the compound is selected from the group consisting of:

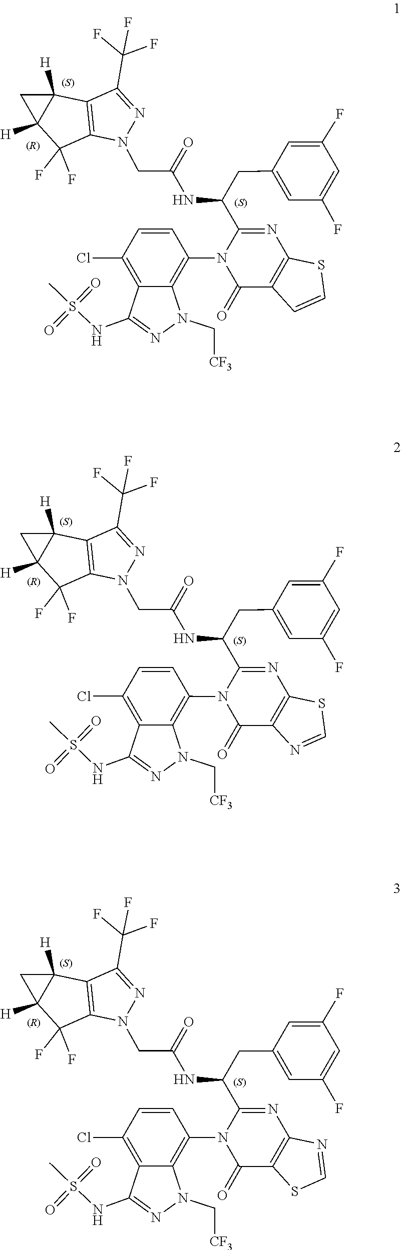

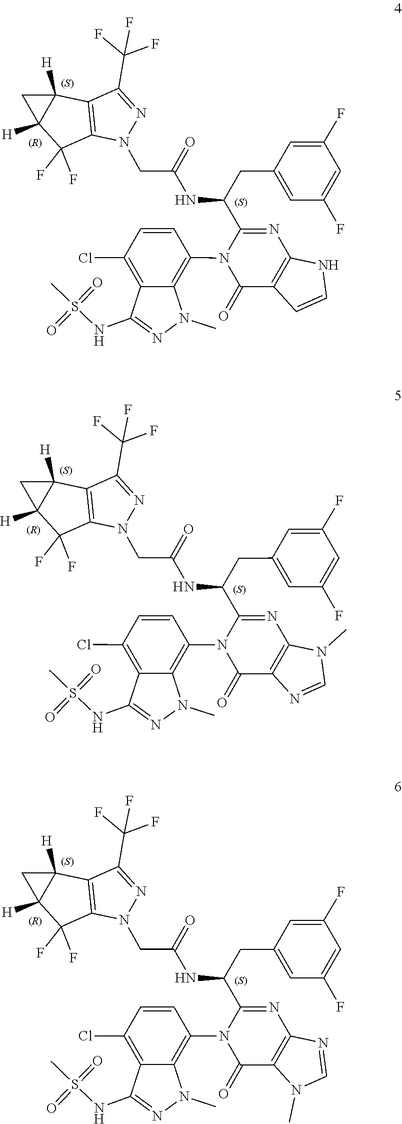

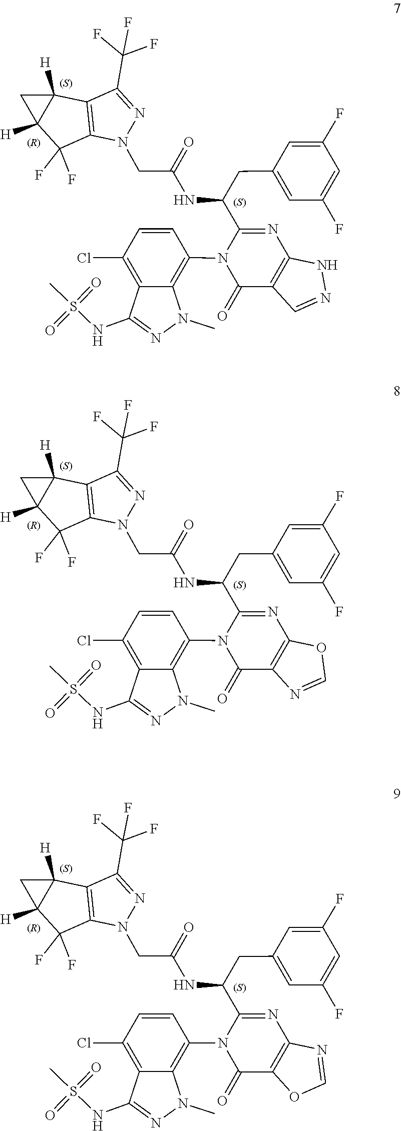

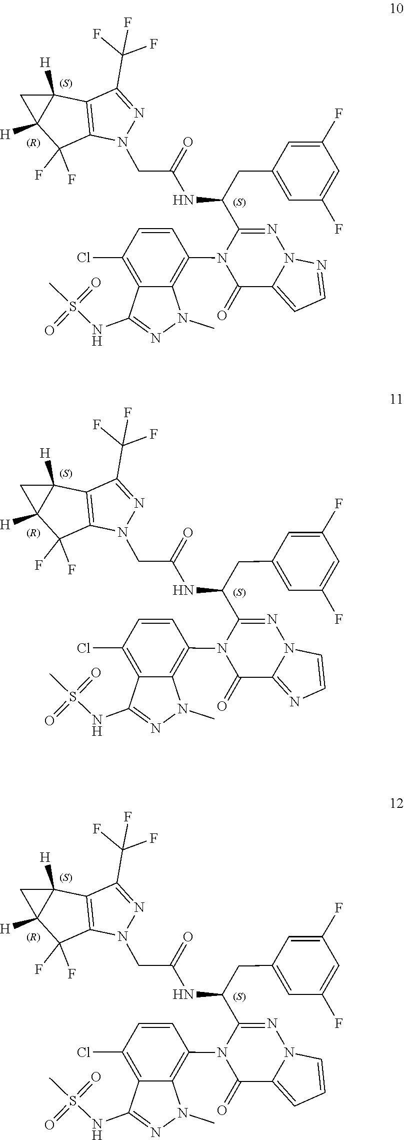

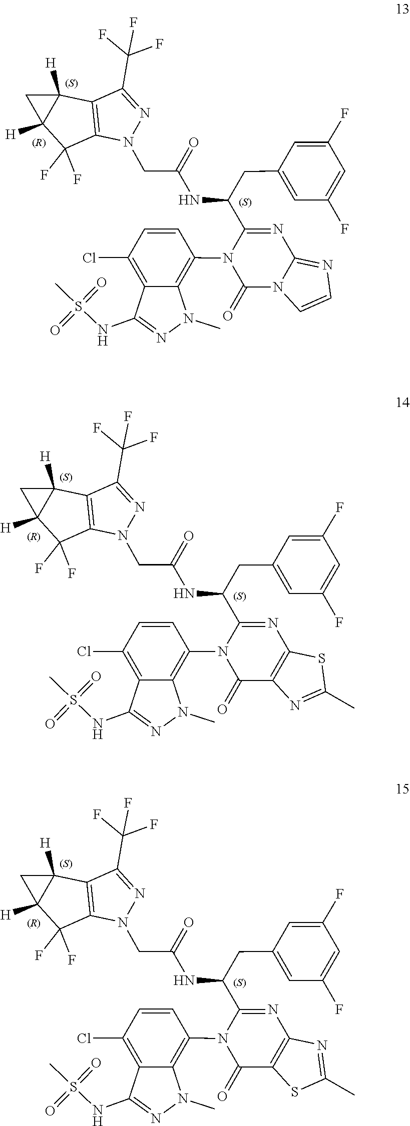

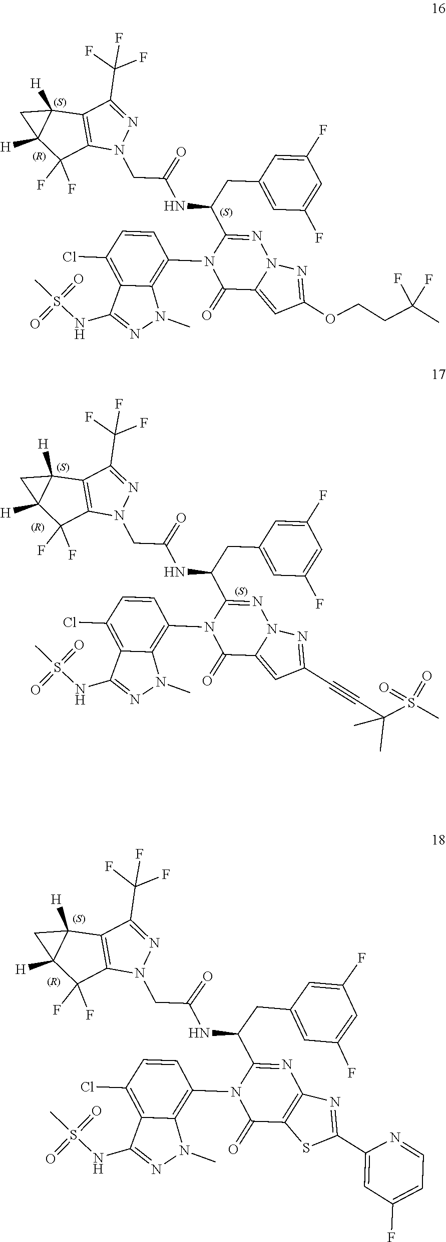

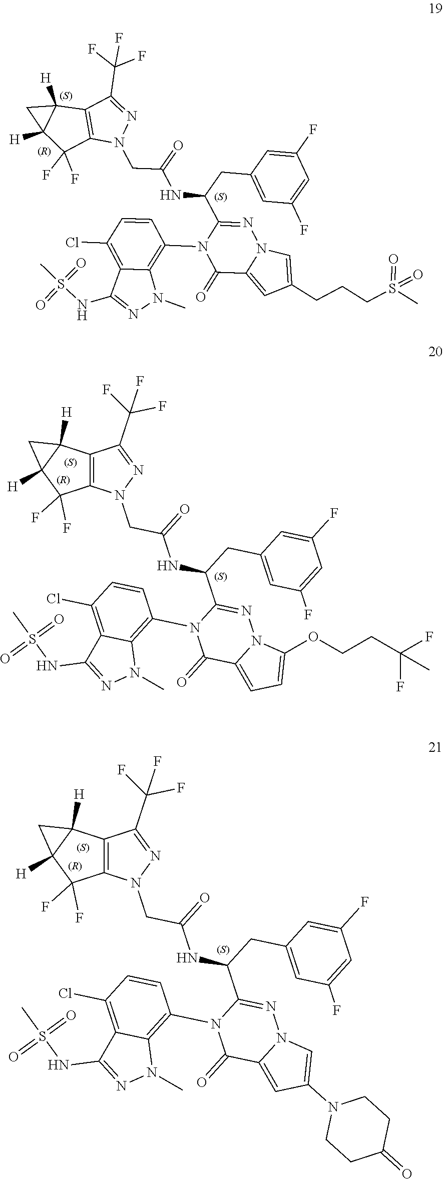

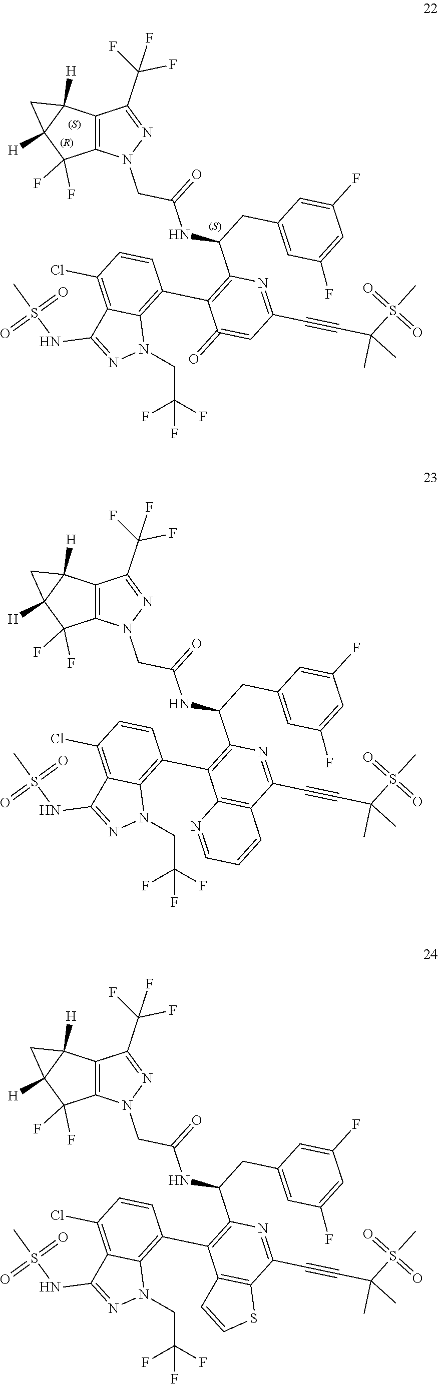

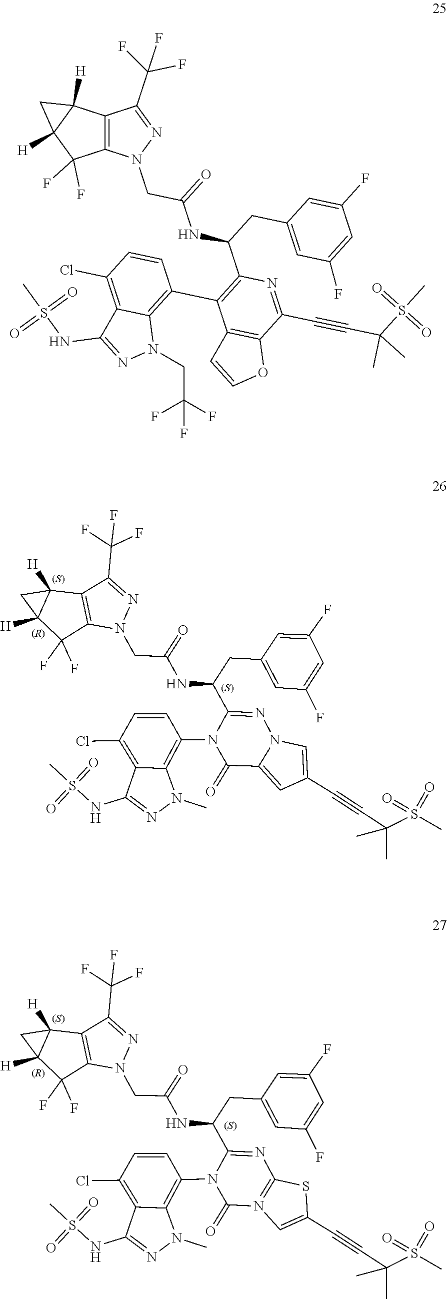

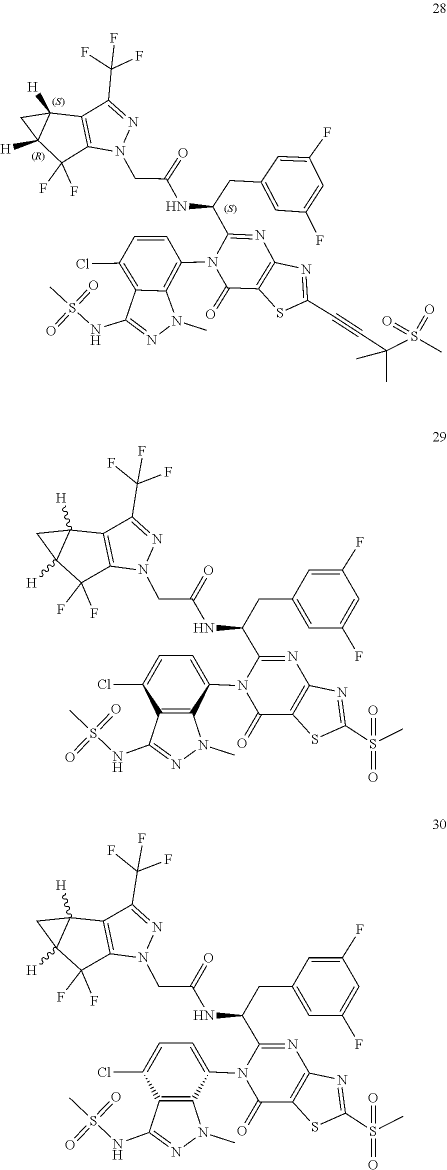

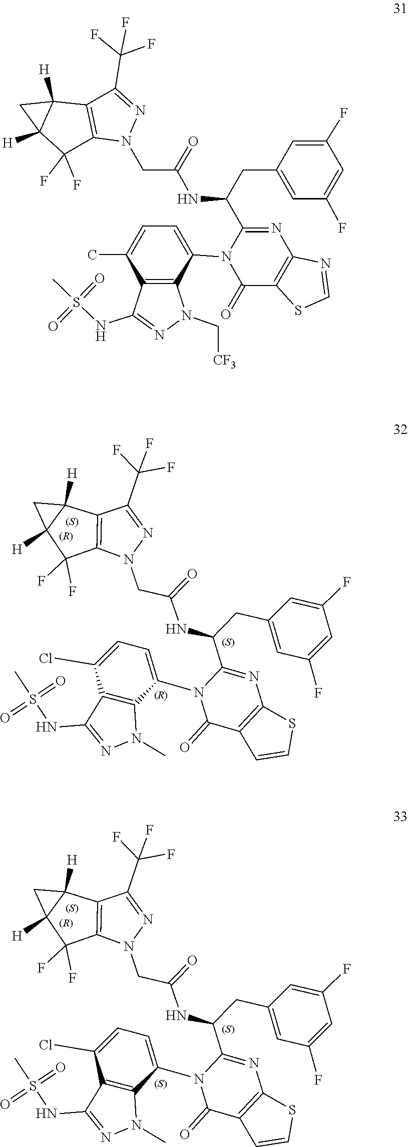

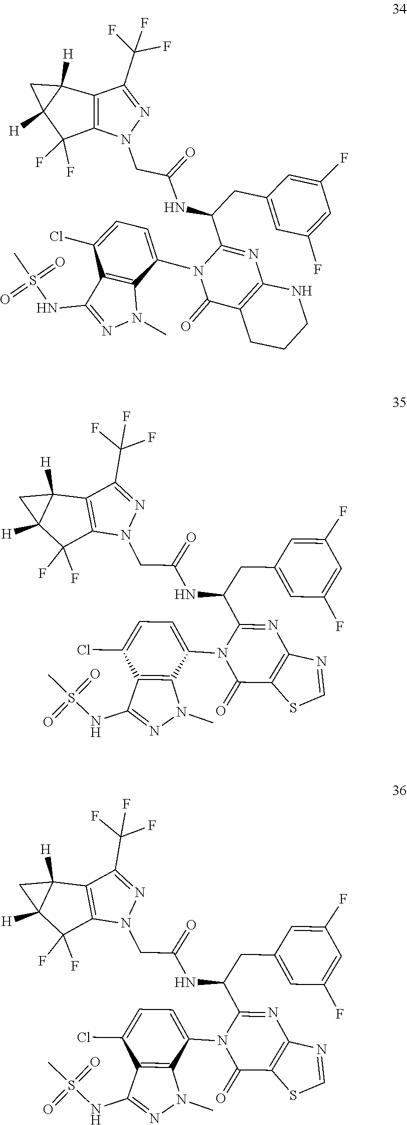

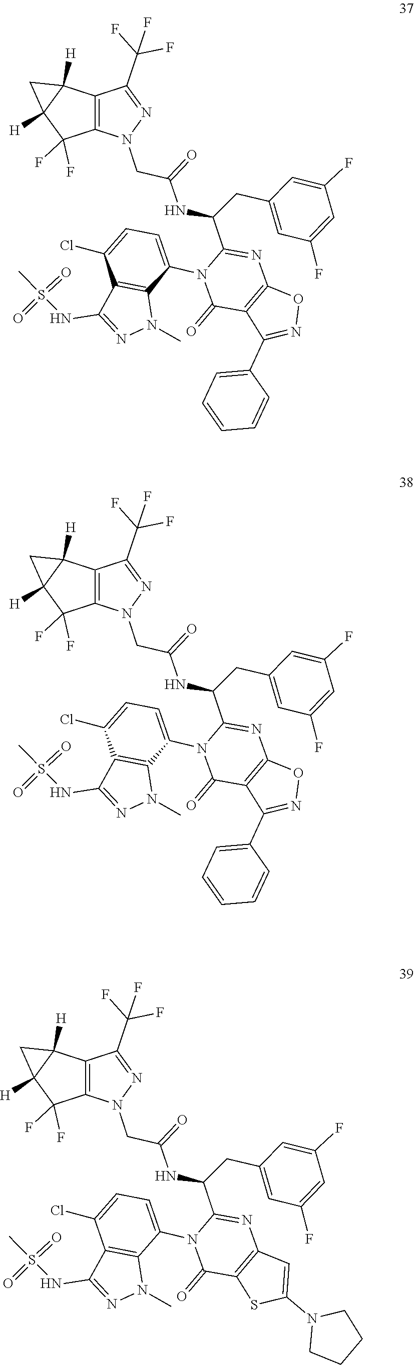

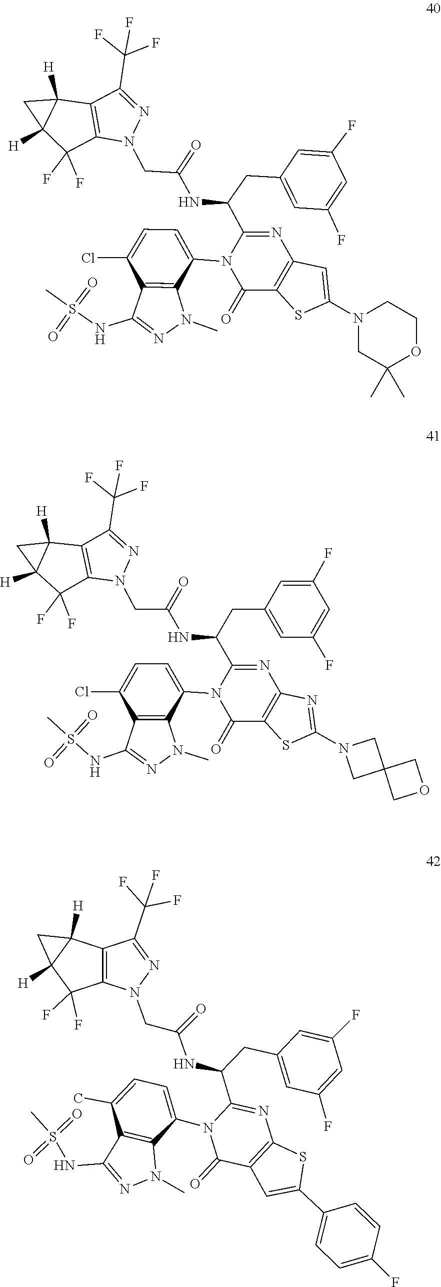

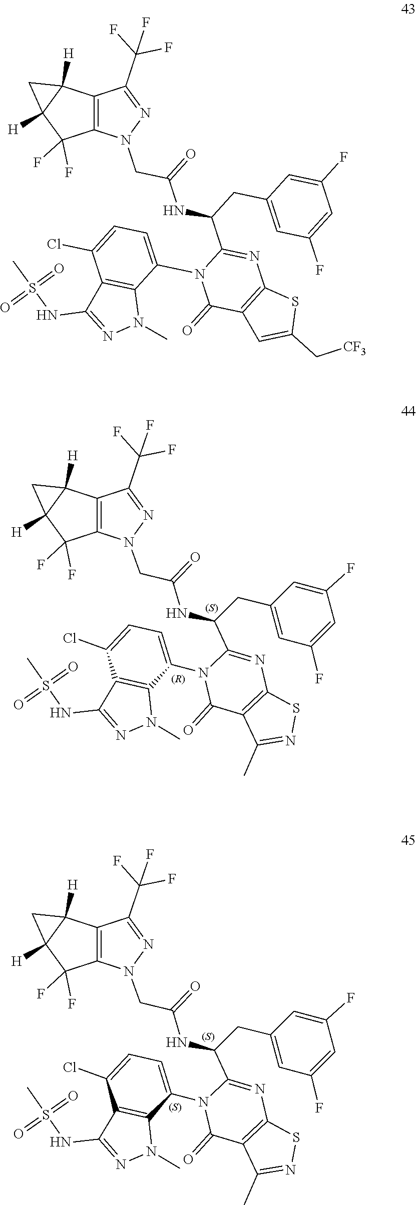

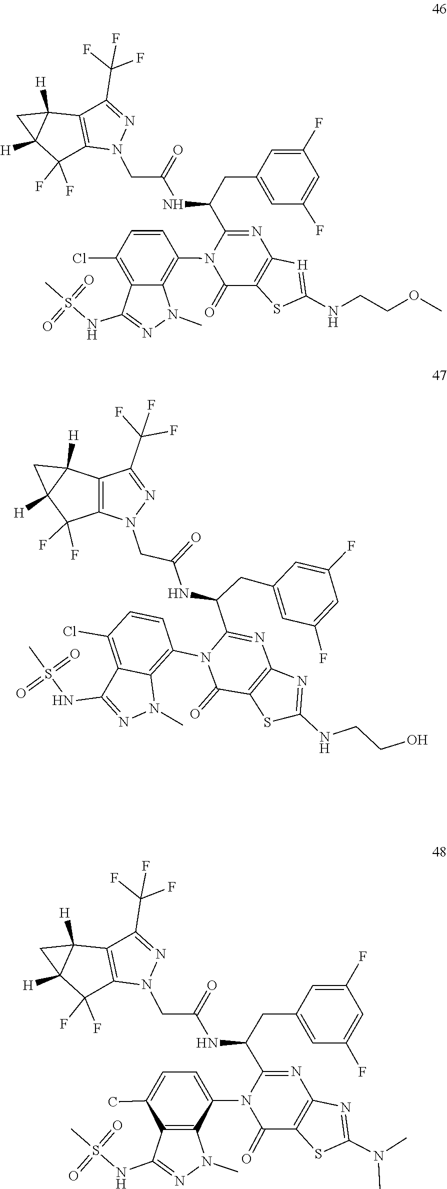

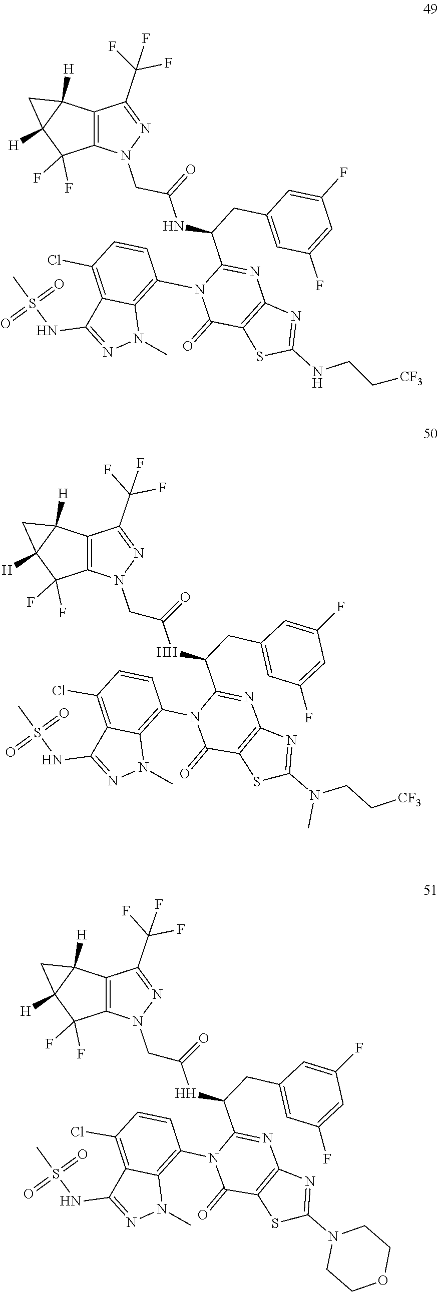

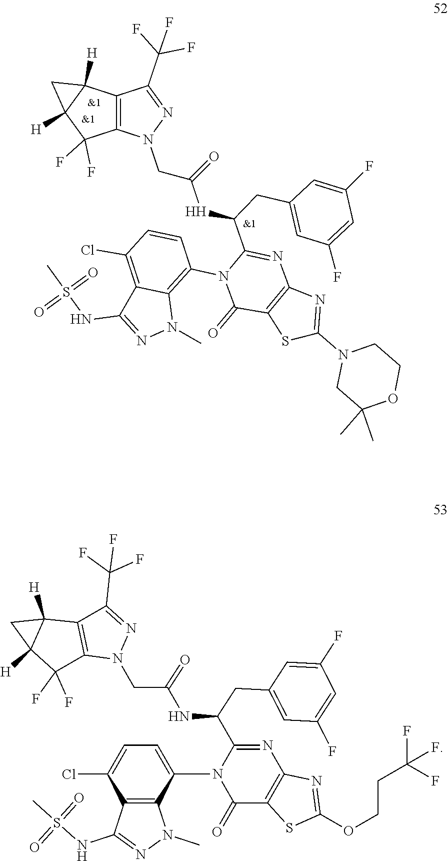

15. A pharmaceutical composition, comprising the compound of claim 1, or a steroisomer thereof, or a pharmaceutically acceptable salt thereof, or a deuterium substitute thereof.

16. The pharmaceutical composition of claim 15, further comprising a pharmaceutically acceptable excipient.

17. The pharmaceutical composition of claim 15, wherein the pharmaceutical composition is formulated for oral administration, intramuscular injection, or subcutaneous injection.

18. A method of treating viral infection in a subject, comprising administering to the subject an effective amount of a compound of claim 1, or a stereoisomer thereof, or a pharmaceutically acceptable salt thereof, or a deuterated compound thereof.

19. The method of claim 18, wherein the viral infection is an HIV infection;
Preferably, further comprising administering to the subject of at least one other therapeutic agent.

20. The method of claim 19, wherein the at least one other therapeutic agent is selected from the group consisting of dolutegravir, bictegravir, lamivudine, fostemsavir, cabotegravir, maraviroc, rilpiverine, atazanavir, tenofovir alafenamide, islatravir, doravirine, and darunavir.
